(19) United States
(12) Patent Application Publication
(10) Pub. No.: US 2023/0233620 A1
(43) Pub. Date: Jul. 27, 2023

(54) HIGH-COMPLEXITY SYNTHETIC GUT BACTERIAL COMMUNITIES

(71) Applicants: Chan Zuckerberg Biohub, Inc., San Francisco, CA (US); The Board of Trustees of the Leland Stanford Junior University, Stanford, CA (US); Federation Bio, Inc., South San Francisco, CA (US)

(72) Inventors: Michael A. Fischbach, Stanford, CA (US); Ariel R. Brumbaugh, San Francisco, CA (US); Alice G. Cheng, San Francisco, CA (US); Dylan Dodd, San Francisco, CA (US); Andres Jesus Aranda-Diaz, San Francisco, CA (US); Min Wang, San Francisco, CA (US); Feiqiao Brian Yu, San Francisco, CA (US); Justin L. Sonnenburg, San Francisco, CA (US); Kerwyn C. Huang, San Francisco, CA (US); Steve Higginbottom, San Francisco, CA (US); Sunit Jain, San Francisco, CA (US); Xiandong Meng, San Francisco, CA (US); Lee Swem, Montara, CA (US); Dante Ricci, Richmond, CA (US); Joshua J. Hamilton, San Mateo, CA (US)

(21) Appl. No.: 17/999,516

(22) PCT Filed: May 21, 2021
(86) PCT No.: PCT/US2021/033762

§ 371 Date: Nov. 21, 2022

Related U.S. Application Data
(60) Provisional application No. 63/028,495, filed on May 21, 2020.

(51) Int. Cl. A61K 35/741; G01N 33/50; C12Q 1/6869; A61K 9/00; A61P 1/00

(52) U.S. Cl. CPC A61K 35/741; G01N 33/5038; C12Q 1/6869; A61K 9/0053; A61P 1/00

(57) ABSTRACT

The present invention provides high-complexity defined gut microbial communities capable of achieving substantial engraftment and having stability following human fecal community microbial challenge and methods of producing the same. Also provided are methods of using high-complexity defined gut microbial communities for the treatment of dysbiosis or a pathological condition in an animal.

### CROSS-REFERENCE TO RELATED APPLICATIONS

This application claims the benefit of and priority to U.S. Provisional Patent Application No. 63/028,495, filed May 21, 2020, the disclosure of which is hereby incorporated by reference in its entirety for all purposes.

### STATEMENT AS TO RIGHTS TO INVENTIONS MADE UNDER FEDERALLY SPONSORED RESEARCH AND DEVELOPMENT

This invention was made with Government support under Grant No: DK113598 awarded by the National Institutes of Health (NIH). The Government has certain rights in the invention.

### BACKGROUND OF THE INVENTION

Fecal microbiota transplantation (FMT) is a promising therapeutic approach that has proved highly effective for treating conditions such as recurrent C. difficile infection (CDI). To avoid the disadvantages of using stool, Allen-Vercoe and Petrof proposed treatment of recurrent CDI using a synthetic bacterial ecosystem of 33 strains developed from a subset of isolates. Allen-Vercoe, E. and Petrof, E O, 2013, “Artificial stool transplantation: progress towards a safer, more effective and acceptable alternative,” Expert Rev. Gastroenterol. Hepatol. 7(4), 291-293 (2013); WO 2013/037068 A1.

FMT has been proposed by Fischbach and colleagues as a therapeutic intervention to change the spectrum of metabolites in a patient's bloodstream, urine, bile and/or feces by engineering the molecular output of the gut bacterial community. Dodd et al., 2017, “A gut bacterial pathway metabolizes aromatic amino acids into nine circulating metabolites,” Nature 551: 648-652; Fischbach M A, 2018, “Microbiome: Focus on Causation and Mechanism,” Cell 174(4):785-790.

Although FMT shows great promise as a therapeutic modality, better transplantable compositions are needed, as are better methods for developing therapeutic agents with a desired activity.

### SUMMARY OF THE INVENTION

In one aspect, provided herein is a high-complexity defined gut microbial community, comprising: a plurality of between 40 and 500 defined microbial strains, wherein the defined microbial strains comprise at least 3 of 4 phyla selected from the group consisting of Bacteroidetes, Firmicutes, Actinobacteria and Proteobacteria. In some embodiments, the defined gut microbial community is capable of: (a) metabolizing at least 90% of enumerated substrates selected from the group consisting of: a-mannan (yeast), acetate, agarose, alanine, alginate, anthocyanin, arabinan, arabinogalactan, arabinoxylan, arginine, asparagine, Aspartate, b-glucans, butyrate, carrageenan, chitin, chlorogenic acids, chondroitin sulfate, cinnamic acid, Cysteine, dextran (40), Dihydrochalcones, Enterodiol, flavan-3-ols, flavanones, flavones, flavonols, folate, formate, galactomannan (carob), galacturonan (homo), galacturonate, glucomannan (konjac), glutamate, Glutamine, Glycine, Histidine, hyaluronan, hydrogen, hydroxycinnamic acids, hydroxyproline, inulin, isoflavones/isoflavanones, Isoleucine, lactate, laminarin, Leucine, levan, Lysine, Methionine, mucin O-linked glycans, Ornithine, Phenylalanine, porphyran, Proline, propionate, rhamnogalacturonan I, rhamnogalacturonan II, Secoisolariciresinol diglucoside, Serine, starch (potato), starch (structure 1), thiamine, Threonine, tryptophan, Tyrosine, Valine, xyloglucan, and xylooligosaccharides (XOS), and/or (b) producing at least 90% of enumerated metabolites selected from the group consisting of: formate, acetate, propionate, butyrate, isobutyrate, valerate, isovalerate, 2-methylbutyrate, caporate, isocaporate, 3-methylvaleric acid, L-phenylalanine, 3-phenylpropionic acid, phyenypyruvate, DL-3-phenyllactic acid, trans-cinnamic acid, phenyllactic acid, phenethylamine, L-tyrosine, 3-(4-hydroxyphenyl)propionic acid, 3-(4-hydroxyphenyl) pyruvic acid, DL-p-hydroxyphenyl lactic acid, p-coumaric acid, 4-hydroxyphenyl acetic acid, tyramine, phenol, p-cresol, 4-ethylphenol, 4-vinylphenol, 4-hydroxybenzoic acid, L-tryptophan, 3-indolepropionic acid, 3-indolepyruvic acid, DL-indole-3-lactic acid, trans-3-indoleacrylic acid, 3-indoleacetic acid, tryptamine, indole, skatol, indole-3-carboxylic acid, indole-3-carboxyaldehyde, N-acetyl-L-phenylalanine, phenylpropionylglycine, 3-(3-hydroxyphenyl) propionic acid, cinnamoylglycine, phenylacetylglycine, phenylacetylglutamine, hippuric acid, 2-hydroxyhippuric acid, 3-hydroxyhippuric acid, 4-hydroxyhippuric acid, 4-hydroxyphenylacetylglycine, phenyl sulfate, phenyl glucuronide, p-cresol sulfate, p-cresol glucuronide, 4-ethylphenol sulfate, 4-ethylphenol glucuronide, N-acetyl-L-tryptophan, 5-hydroxy-L-typtophan, N-acetyl serotonin, 3-indolepriopionylglycine, indolyl acryloylglycine, indoxyl sulfate, indoxyl glucuronide, 5-hydroxyindole-3-acetic acid, indoleacetylglycine, lithocholic acid, murocholic acid, ursodeoxycholic acid, hyodeoxycholic acid, deoxycholic acid, 7-oxocholic acid, ω-muricholic acid, α-muricholic acid, β-muricholic acid, γ-muricholic acid, 7βcholic acid, taurolithocholic acid, tauroursodeoxycholic acid, taurohyodeoxycholic acid, taurochenodeoxycholic acid, taurodeoxycholic acid, tauro-β-muricholic acid, tauro-ω-muricholic acid, and taurocholic acid. In some embodiments, the high-complexity defined gut microbial community achieves substantial engraftment when administered to a gnotobiotic mouse. In some embodiments, the engrafted high-complexity defined gut microbial community is stable following a human fecal community microbial challenge.

In some embodiments, metabolization of a substrate and/or production of a metabolite can be determined by culturing the defined gut microbial community in vitro and measuring whether the substrate is metabolized and/or the metabolite is produced by liquid chromatography-mass spectrometry analysis. In some embodiments, metabolization of a substrate and/or production of a product can be determined by administering the defined gut microbial community to a gnotobiotic mouse and measuring whether the substrate is metabolized and/or the product is produced after a defined period of time by liquid chromatography-mass spectrometry analysis of a sample obtained from the mouse. In certain embodiments, the defined gut microbial community is administered via a route selected from the group consisting of oral, rectal, fecal (by enema), and naso/oro-gastric gavage. In certain embodiments, the defined period of time is about 1 hour, 2 hours, 4 hours, 8 hours, 12 hours, 24 hours, 2 days, 7 days, 14 days, 1 month, or 2 months. In certain embodiments, the sample is selected from the group consisting of a fecal sample, urine sample, blood sample, or serum sample.

In another aspect, provided herein is a high-complexity defined gut microbial community, comprising: a plurality of between 40 and 500 defined microbial strains, wherein the defined microbial strains comprise at least 3 of 4 phyla selected from the group consisting of Bacteroidetes, Firmicutes, Actinobacteria and Proteobacteria; wherein the defined gut microbial community encode the enzymes catalyzing all reactions for at least 90% of the enumerated MetaCyc metabolic pathways selected from the group consisting of: 1CMET2-PWY, 2.6.1.32-RXN, AEROBACTINSYN-PWY, ALACAT2-PWY, ALADEG-PWY, ALANINE-DEG3-PWY, ALANINE-SYN2-PWY, ALANINE-VALINESYN-PWY, ANAPHENOXI-PWY, ARGASEDEG-PWY, ARGDEG-III-PWY, ARGDEG-IV-PWY, ARGDEGRAD-PWY, ARGDEG-V-PWY, ARG-GLU-PWY, ARGININE-SYN4-PWY, ARG-PRO-PWY, ARGSYNBSUB-PWY, ARGSYN-PWY, ASPARAGINE-BIOSYNTHESIS, ASPARAGINE-DEG1-PWY, ASPARAGINE-DEG1-PWY-1, ASPARAGINESYN-PWY, ASPARTATE-DEG1-PWY, ASPARTATESYN-PWY, ASPASN-PWY, ASPSYNII-PWY, AST-PWY, BETA-ALA-DEGRADATION-I-PWY, CAMALEXIN-SYN, CITRULBIO-PWY, CITRULLINE-DEG-PWY, COA-PWY, CODH-PWY, CYSTEINE-DEG-PWY, CYSTSYN-PWY, DAPLYSINESYN-PWY, ENTBACSYN-PWY, ETHYL-PWY, FAO-PWY, FERMENTATION-PWY, GLNSYN-PWY, GLUDEG-I-PWY, GLUGLNSYN-PWY, GLUTAMATE-DEG1-PWY, GLUTAMATE-SYN2-PWY, GLUTAMINDEG-PWY, GLUTAMINEFUM-PWY, GLUTATHIONESYN-PWY, GLUTDEG-PWY, GLUTORN-PWY, GLUTSYNIII-PWY, GLUTSYN-PWY, GLYCGREAT-PWY, GLYSYN-ALA-PWY, GLYSYN-PWY, GLYSYN-THR-PWY, HISDEG-PWY, HISHP-PWY, HISTDEG-PWY, HISTSYN-PWY, HOMOCYSDEGR-PWY, HOMOSER-METSYN-PWY, HOMOSERSYN-PWY, HSERMETANA-PWY, HYDROXYPRODEG-PWY, ILEUDEG-PWY, ILEUSYN-PWY, LARABITOLUTIL-PWY, LCYSDEG-PWY, LEU-DEG2-PWY, LEUSYN-PWY, LYSDEGII-PWY, LYSINE-AMINOAD-PWY, LYSINE-DEG1-PWY, MALATE-ASPARTATE-SHUTTLE-PWY, METH-ACETATE-PWY, METHANOGENESIS-PWY, METHIONINE-DEG1-PWY, MGLDLCTANA-PWY, ORN-AMINOPENTANOATE-CAT-PWY, ORNDEG-PWY, P101-PWY, P162-PWY, P163-PWY, P181-PWY, P261-PWY, P283-PWY, P401-PWY, P541-PWY, PHENYLALANINE-DEG1-PWY, PHESYN, PHOSLIPSYN2-PWY, PHOSPHONOTASE-PWY, PROSYN-PWY, PROUT-PWY, PWY0-1021, PWY0-1221, PWY0-1299, PWY0-1303, PWY0-1305, PWY0-1313, PWY0-1317, PWY0-1321, PWY0-1338, PWY0-1347, PWY0-1355, PWY0-1356, PWY0-1534, PWY0-1544, PWY0-1565, PWY0-1576, PWY0-1577, PWY0-1578, PWY0-1585, PWY0-1601, PWY0-42, PWY0-461, PWY0-823, PWY0-901, PWY-1, PWY-1061, PWY-1121, PWY-1186, PWY1-2, PWY-1263, PWY-1622, PWY-1722, PWY-1781, PWY-181, PWY-1881, PWY-1962, PWY-1981, PWY1F-467, PWY1F-FLAVSYN, PWY1G-0, PWY-2021, PWY-2161, PWY-2181, PWY-2201, PWY-2821, PWY-2941, PWY-2942, PWY-3, PWY-3022, PWY-3081, PWY-3161, PWY-3162, PWY-3181, PWY-3301, PWY-3341, PWY-3385, PWY-3461, PWY-3462, PWY-3581, PWY-361, PWY-3661, PWY-3661-1, PWY-381, PWY-3841, PWY-3941, PWY3DJ-12, PWY3O-4107, PWY3O-4108, PWY-4, PWY-40, PWY-4002, PWY-4041, PWY-4201, PWY-4281, PWY-43, PWY-4321, PWY-4341, PWY-4361, PWY-46, PWY490-3, PWY490-4, PWY-4981, PWY-4983, PWY-4984, PWY4FS-6, PWY-5, PWY-5022, PWY-5024, PWY-5028, PWY-5029, PWY-5030, PWY-5031, PWY-5041, PWY-5048, PWY-5049, PWY-5057, PWY-5075, PWY-5076, PWY-5078, PWY-5079, PWY-5081, PWY-5082, PWY-5087, PWY-5097, PWY-5101, PWY-5103, PWY-5104, PWY-5108, PWY-5109, PWY-5129, PWY-5135, PWY-5136, PWY-5151, PWY-5154, PWY-5155, PWY-5159, PWY-5176, PWY-5188, PWY-5189, PWY-5196, PWY-5199, PWY-5207, PWY-5250, PWY-5254, PWY-5265, PWY-5280, PWY-5283, PWY-5290, PWY-5297, PWY-5298, PWY-5311, PWY-5314, PWY-5316, PWY-5319, PWY-5324, PWY-5329, PWY-5331, PWY-5332, PWY-5364, PWY-5381, PWY-5382, PWY-5386, PWY-5394, PWY-5399, PWY-5436, PWY-5437, PWY-5441, PWY-5443, PWY-5458, PWY-5467, PWY-5468, PWY-5473, PWY-5474, PWY-5494, PWY-5497, PWY-5499, PWY-5629, PWY-5651, PWY-5653, PWY-5665, PWY-5669, PWY-5675, PWY-5679, PWY-5686, PWY-5710, PWY-5736, PWY-5737, PWY-5739, PWY-5740, PWY-5742, PWY-5747, PWY-5748, PWY-5751, PWY-5754, PWY-5766, PWY-5770, PWY-5784, PWY-5788, PWY-5797, PWY-5800, PWY-581, PWY-5811, PWY-5818, PWY-5826, PWY-5877, PWY-5883, PWY-5886, PWY-5912, PWY-5913, PWY-5921, PWY-5936, PWY-5940, PWY-5958, PWY-5963, PWY-5968, PWY-5978, PWY-5980, PWY-5990, PWY-6003, PWY-6004, PWY-601, PWY-6030, PWY-6039, PWY-6045, PWY-6052, PWY-6053, PWY-6054, PWY-6055, PWY-6068, PWY-6069, PWY-6082, PWY-6120, PWY-6121, PWY-6122, PWY-6123, PWY-6124, PWY-6133, PWY-6134, PWY-6141, PWY-6143, PWY-6148, PWY-6151, PWY-6160, PWY-6173, PWY-6196, PWY-6219, PWY-622, PWY-6220, PWY-6233, PWY-6273, PWY-6277, PWY-6281, PWY-6307, PWY-6309, PWY-6313, PWY-6318, PWY-6320, PWY-6321, PWY-6322, PWY-6324, PWY-6328, PWY-6334, PWY-6339, PWY-6343, PWY-6344, PWY-6345, PWY-6346, PWY-6375, PWY-6376, PWY-6381, PWY-6386, PWY-6387, PWY-6397, PWY-6403, PWY-6407, PWY-6408, PWY-6409, PWY-6431, PWY-6435, PWY-6444, PWY-6455, PWY-6456, PWY-6457, PWY-6466, PWY-6471, PWY-6473, PWY-6481, PWY-6486, PWY-6493, PWY-6495, PWY-6511, PWY-6512, PWY-6519, PWY-6533, PWY-6535, PWY-6536, PWY-6537, PWY-6543, PWY-6549, PWY-6559, PWY-6562, PWY-6572, PWY-6573, PWY-6574, PWY-6578, PWY-6588, PWY-6614, PWY-6627, PWY66-301, PWY66-375, PWY-6638, PWY66-391, PWY-6642, PWY66-420, PWY66-421, PWY66-425, PWY66-426, PWY66-428, PWY-6643, PWY-6661, PWY-6673, PWY-6682, PWY-6690, PWY-6696, PWY-6711, PWY-6717, PWY-6720, PWY-6724, PWY-6728, PWY-6731, PWY-6735, PWY-6749, PWY-6769, PWY-6771, PWY-6772, PWY-6773, PWY-6781, PWY-6784, PWY-6790, PWY-6791, PWY-6802, PWY-6807, PWY-6808, PWY-6813, PWY-6815, PWY-6816, PWY-6817, PWY-6818, PWY-6821, PWY-6822, PWY-6823, PWY-6831, PWY-6832, PWY-6834, PWY-6840, PWY-6845, PWY-6853, PWY-6854, PWY-6855, PWY-6891, PWY-6892, PWY-6896, PWY-6898, PWY-6902, PWY-6907, PWY-6908, PWY-6920, PWY-6922, PWY-6936, PWY-6942, PWY-6949, PWY-6953, PWY-6955, PWY-6963, PWY-6964, PWY-6965, PWY-6968, PWY-6969, PWY-6981, PWY-6982, PWY-6986, PWY-6994, PWY-7000, PWY-701, PWY-7014, PWY-7015, PWY-7016, PWY-7018, PWY-7019, PWY-702, PWY-7022, PWY-7025, PWY-7028, PWY-7040, PWY-7046, PWY-7052, PWY-7054, PWY-7064, PWY-7072, PWY-7088, PWY-7090, PWY-7097, PWY-7104, PWY-7115, PWY-7117, PWY-7118, PWY-7126, PWY-7147, PWY-7153, PWY-7158, PWY-7176, PWY-7177, PWY-7185, PWY-7186, PWY-7219, PWY-7221, PWY-7234, PWY-7246, PWY-7248, PWY-7250, PWY-7255, PWY-7274, PWY-7275, PWY-7282, PWY-7288, PWY-7297, PWY-7304, PWY-7315, PWY-7316, PWY-7318, PWY-7342, PWY-7351, PWY-7356, PWY-7376, PWY-7377, PWY-7383, PWY-7387, PWY-7397, PWY-7398, PWY-7400, PWY-7414, PWY-7425, PWY-7430, PWY-7432, PWY-7440, PWY-7441, PWY-7456, PWY-7467, PWY-7498, PWY-7501, PWY-7506, PWY-7510, PWY-7514, PWY-7518, PWY-7520, PWY-7525, PWY-7531, PWY-7532, PWY-7533, PWY-7536, PWY-7542, PWY-7543, PWY-7547, PWY-7549, PWY-7550, PWY-7555, PWY-7561, PWY-7565, PWY-7570, PWY-7571, PWY-7600, PWY-7605, PWY-761, PWY-7612, PWY-7626, PWY-7645, PWY-7648, PWY-7649, PWY-7650, PWY-7665, PWY-7667, PWY-7668, PWY-7669, PWY-7671, PWY-7674, PWY-7688, PWY-7690, PWY-7693, PWY-7694, PWY-7701, PWY-7704, PWY-7706, PWY-7708, PWY-7717, PWY-7718, PWY-7719, PWY-7733, PWY-7734, PWY-7735, PWY-7737, PWY-7751, PWY-7761, PWY-7765, PWY-7767, PWY-7769, PWY-7770, PWY-7782, PWY-7790, PWY-7791, PWY-7793, PWY-7797, PWY-7811, PWY-7814, PWY-7822, PWY-7824, PWY-7826, PWY-7842, PWY-7850, PWY-7851, PWY-7855, PWY-7860, PWY-7861, PWY-7863, PWY-7867, PWY-7870, PWY-7880, PWY-7888, PWY-7889, PWY-7891, PWY-7892, PWY-7897, PWY-7901, PWY-7904, PWY-7907, PWY-7909, PWY-7910, PWY-7913, PWY-7917, PWY-7930, PWY-7931, PWY-7936, PWY-7953, PWY-7955, PWY-7956, PWY-7957, PWY-7958, PWY-7959, PWY-7960, PWY-7962, PWY-7977, PWY-7985, PWY-7986, PWY-7987, PWY-7988, PWY-7990, PWY-8002, PWY-8003, PWY-8006, PWY-8007, PWY-8008, PWY-8009, PWY-801, PWY-8010, PWY-8011, PWY-8013, PWY-8014, PWY-8015, PWY-8016, PWY-8017, PWY-8024, PWY-8032, PWY-8040, PWY-8043, PWY-8045, PWY-8071, PWY-8072, PWY-8080, PWY-8081, PWY-8082, PWY-8083, PWY-8086, PWY-8088, PWY-842, PWY-861, PWY-862, PWY8J2-1, PWY8J2-22, PWYDQC-4, PWYG-321, PWY-I9, PWYQT-4450, PWYQT-4476, PYRIDNUCSAL-PWY, PYRIDNUCSYN-PWY, PYRIDOXSYN-PWY, SAM-PWY, SERDEG-PWY, SERSYN-PWY, SPHINGOLIPID-SYN-PWY, TAURINEDEG-PWY, THRDLCTCAT-PWY, THREONINE-DEG2-PWY, TRNA-CHARGING-PWY, TRPCAT-PWY, TRPIAACAT-PWY, TRPKYNCAT-PWY, TRPSYN-PWY, TRYPDEG-PWY, TYRFUMCAT-PWY, TYRSYN, UDPNACETYLGALSYN-PWY, UDPNAGSYN-PWY, VALDEG-PWY, and VALSYN-PWY.

In some embodiments, encoding the enzymes catalyzing all reactions of a MetaCyc metabolic pathway can be determined by culturing the defined gut microbial community in vitro and measuring whether a substrate in the pathway is metabolized, a metabolite in the pathway is produced, and/or a reaction intermediate in the pathway is produced by liquid chromatography-mass spectrometry analysis. In some embodiments, encoding the enzymes catalyzing all reactions of a MetaCyc metabolic pathway can be determined by administering the defined gut microbial community to a gnotobiotic mouse and measuring whether a substrate in the pathway is metabolized, a metabolite in the pathway is produced, and/or a reaction intermediate in the pathway is produced after a defined period of time by liquid chromatography-mass spectrometry analysis of a sample obtained from the mouse. In certain embodiments, the defined gut microbial community is administered via a route selected from the group consisting of oral, rectal, fecal (by enema), and naso/oro-gastric gavage. In certain embodiments, the defined period of time is about 1 hour, 2 hours, 4 hours, 8 hours, 12 hours, 24 hours, 2 days, 7 days, 14 days, 1 month, or 2 months. In certain embodiments, the sample is selected from the group consisting of a fecal sample, urine sample, blood sample, or serum sample.

In some embodiments, the at least 3 of 4 phyla comprise Bacteroidetes, Firmicutes, and Actinobacteria. In certain embodiments, the high complexity defined gut microbial community comprises Bacteroidetes, Firmicutes, Actinobacteria and Proteobacteria. In certain embodiments, the defined microbial strains comprise phyla selected from the group consisting of Bateriodales, Clostridiales, Lactobacillales, Negativicutes, Eggerthellales, Bifidobacteriales, and Proteobacteria.

In some embodiments, the defined microbial strains comprise a genus selected from the group consisting of: Acidaminococcus, Adlercreutzia, Akkermansia, Alistipes, Anaerobutyricum, Anaerofustis, Anaerostipes, Anaerotruncus, Bacteroides, Parabacteroides, Bifidobacterium, Bilophila, Blautia, Catenibacterium, Clostridium, Tyzzerella, Absiella, Collinsella, Coprococcus, Dialister, Eubacterium, Holdemanella, Intestinibacter, Megasphaera, Odoribacter, Parabacteroides, Granulicatella, Holdemania, Hungatella, Intestinimonas, Solobacterium, Mitsuokella, Olsenella, Parabacteroides, Prevotella, Roseburia, Ruminococcus, Slackia, Butyrivibrio, Subdoligranulum, Turicibacter, Butyricimonas, Streptococcus, Dorea, Oscillibacter, Desulfovibrio, Ethanoligenens, Marvinbryantia, Lactobacillus, and Faecalibacterium.

In some embodiments, the defined microbial strains are selected from the group consisting of: Acidaminococcus fermentans, Acidaminococcus sp., Adlercreutzia equolifaciens, Akkermansia muciniphila, Alistipes finegoldii, Alistipes indistinctus, Alistipes onderdonkii, Anaerobutyricum hallii, Anaerofustis stercorihominis, Anaerostipes caccae, Anaerotruncus colihominis, Bacteroides caccae, Bacteroides cellulosilyticus, Bacteroides coprocola, Bacteroides coprophilus, Bacteroides dorei, Bacteroides eggerthii, Bacteroides finegoldii, Bacteroides fragilis, Bacteroides intestinalis, Bacteroides ovatus, Bacteroides rodentium, Bacteroides thetaiotaomicron, Bacteroides xylanisolvens, Parabacteroides distasonis, Bacteroides dorea, Bacteroides stercoris, Bacteroides uniformis, Bacteroides vulgatus, Bifidobacterium pseudocatenulatum, Bilophila wadsworthia, Blautia hansenii, Blautia hydrogenotrophica, Blautia obeum, Blautia sp., Blautia wexlerae, Catenibacterium mitsuokai, Clostridium asparagiforme, Clostridium hylemonae, Clostridium leptum, Tyzzerella nexilis, Clostridium saccharolyticum, Absiella dolichum, Collinsella aerofaciens, Collinsella stercoris, Coprococcus comes, Dialister invisus, Eubacterium rectale, Eubacterium siraeum, Eubacterium ventriosum, Coprococcus eutactus, Holdemanella biformis, Intestinibacter bartlettii, Megasphaera sp., Odoribacter splanchnicus, Parabacteroides merdae, Parabacteroides sp., Granulicatella adiacens, Holdemania filiformis, Hungatella hathewayi, Intestinimonas butyriciproducens, Solobacterium moorei, Mitsuokella multacida, Olsenella uli, Parabacteroides johnsonii, Prevotella buccalis, Prevotella copri, Roseburia inulinivorans, Clostridium sp., Ruminococcus gauvreauii, Ruminococcus lactaris, Ruminococcus torques, Alistipes putredinis, Alistipes senegalensis, Clostridium spiroforme, Slackia exigua, Bacteroides pectinophilus, Butyrivibrio crossotus, Subdoligranulum variabile, Turicibacter sanguinis, Bifidobacterium breve, Bifidobacterium catenulatum, Butyricimonas virosa, Streptococcus salivarius subsp. thermophilus, Dorea formicigenerans, Bacteroides plebeius, Ruminococcus gnavus, Oscillibacter sp., Clostridium sp., Slackia heliotrinireducens, Desulfovibrio piger, Clostridium methylpentosum, Ethanoligenens harbinense, Marvinbryantia formatexigens, Lactobacillus ruminis, Clostridium bolteae, Clostridium hiranonis, Clostridium scindens, Clostridium sp., Clostridium orbiscindens, Alistipes shahii, and Faecalibacterium prausnitzii.

In certain embodiments, the defined microbial strains are selected from the group consisting of: Acidaminococcus fermentans—VR4, Acidaminococcus sp.—D21, Adlercreutzia equolifaciens—FJC-B9, Akkermansia muciniphila—Muc [CIP 107961], Alistipes finegoldii—AHN 2437, Alistipes indistinctus—JCM 16068, YIT 12060, Alistipes onderdonkii—WAL 8169, Anaerobutyricum hallii—VPI B4-27, Anaerofustis stercorihominis—ATCC BAA-858, CCUG 47767, CIP 108481, WAL 14563, Anaerostipes caccae—L1-92, Anaerotruncus colihominis —277, Bacteroides caccae—VPI 3452A [CIP 104201T, JCM 9498], Bacteroides cellulosilyticus—CRE21, CCUG 44979, Bacteroides coprocola—M16, Bacteroides coprophilus—CB42, JCM 13818, Bacteroides dorei —175, Bacteroides dorei —5_1_36/D4, Bacteroides eggerthii—ATCC 27754, NCTC 11155, Bacteroides finegoldii —199, Bacteroides fragilis —3_1_12, Bacteroides intestinalis —341, Bacteroides ovatus—NCTC 11153, Bacteroides rodentium—ST28, CCUG 59334, JCM 16469, Bacteroides thetaiotaomicron —1_1_6, Bacteroides fragilis —2_1_16, Bacteroides xylanisolvens —2_1_22, Parabacteroides distasonis —3_1_19, Bacteroides dorea —9_1_42FAA, Bacteroides ovatus—D2, Bacteroides stercoris—VPI B3-21, ATCC 43183, CIP 104203, JCM 9496, Bacteroides thetaiotaomicron—VPI 5482 [CIP 104206T, E50, NCTC 10582], Bacteroides uniformis—ATCC 8492, Bacteroides vulgatus—NCTC 11154, Bifidobacterium pseudocatenulatum—B1279, ATCC 27919, Bilophila wadsworthia—WAL 7959 [Lab 88-130H], Blautia hansenii—VPI C7-24, Blautia hydrogenotrophica—S5a33, Blautia obeum—ATCC 29174, KCTC 15206, VPI B3-21, Blautia sp.—KLE 1732, Blautia wexlerae—ATCC BAA-1564, JCM 17041, KCTC 5965, WAL 14507, Catenibacterium mitsuokai—RCA14-39, CIP 106738, JCM 10609, Clostridium asparagiforme—N6, CCUG 48471, Clostridium hylemonae—TN-271, JCM 10539, Clostridium leptum—VPI T7-24-1, ATCC 29065, Tyzzerella nexilis DSM 1787, Clostridium saccharolyticum—WM1, ATCC 35040, NRC 2533, Absiella dolichum DSM 3991, Collinsella aerofaciens—VPI 1003 [DSM 3979, JCM 10188], Collinsella stercoris—RCA 55-54, JCM 10641, Coprococcus comes—VPI CI-38, Dialister invisus—E7.25, CCUG 47026, Eubacterium rectale—VPI 0990 [CIP 105953], Eubacterium siraeum—VPI T9-50-2, ATCC 29066, DSM 3996, Eubacterium ventriosum—VPI 1013B, Coprococcus eutactus—VPI C33-22, Holdemanella biformis—VPI C17-5, ATCC 27806, KCTC 5969, Intestinibacter bartlettii—WAL 16138, ATCC BAA-827, CCUG 48940, Megasphaera sp.—Sanger 24, Sanger_24, Odoribacter splanchnicus—1651/6, ATCC 29572, CCUG 21054, CIP 104287, LMG 8202, NCTC 10825, Parabacteroides distasonis—NCTC 11152, Parabacteroides merdae—VPI T4-1, ATCC 43184, CCUG 38734, CIP 104202, JCM 9497, Parabacteroides sp.—D13, Granulicatella adiacens—GaD [CIP 103243, DSM 9848], Holdemania filiformis—VPI J1-31B-1, ATCC 51649, Hungatella hathewayi—1313, CCUG 43506, CIP 109440, MTCC 10951, Intestinimonas butyriciproducens—SRB-521-5-1, CCUG 63529, Solobacterium moorei—RCA59-74, CIP 106864, JCM 10645, Mitsuokella multacida—A 405-1, ATCC 27723, NCTC 10934, Olsenella uli—D76D-27C, ATCC 49627, CIP 109912, Parabacteroides johnsonii—M-165, CIP 109537, JCM 13406, Prevotella buccalis—HS4, ATCC 35310, NCDO 2354, Prevotella copri—CB7, JCM 13464, Roseburia inulinivorans—A2-194, CIP 109405, JCM 17584, NCIMB 14030, Clostridium sp.—VPI C48-50 (unassigned Clostridiales), Ruminococcus gauvreauii—CCRI-16110, CCUG 54292, JCM 14987, NML 060141, Ruminococcus lactaris—VPI X6-29, Ruminococcus torques—VPI B2-51, Alistipes putredinis—CCUG 45780, CIP 104286, ATCC 29800, Carlier 10203, VPI 3293, Alistipes senegalensis—CSUR P150, JCM 32779, JC50, Clostridium spiroforme—VPI C28-23-1A, ATCC 29900, NCTC 11211, Slackia exigua—S-7, ATCC 700122, JCM 11022, KCTC 5966, Bacteroides pectinophilus—N3, Butyrivibrio crossotus—T9-40A, ATCC 29175, Subdoligranulum variabile—BI-114, CCUG 47106, Turicibacter sanguinis—MOL361, NCCB 100008, Bifidobacterium breve—S1, ATCC 15700, NCTC 11815, Bifidobacterium catenulatum—B669, ATCC 27539, CECT 7362, CIP 104175, DSM 20103, Butyricimonas virosa—MT12, CCUG 56611, JCM 15149, Streptococcus salivarius subsp. thermophilus—LMD-9, Dorea formicigenerans—VPI C8-13 [JCM 9500], Bacteroides plebeius—M12, Ruminococcus gnavus—VPI C7-9, Oscillibacter sp.—KLE 1728, Clostridium sp.—M62/1, Slackia heliotrinireducens—RHS 1, ATCC 29202, NCTC 11029, Desulfovibrio piger—VPI C3-23 [DSM 749], Clostridium methylpentosum—R2, ATCC 43829, Ethanoligenens harbinense—YUAN-3, CGMCC 1.5033, JCM 12961, Marvinbryantia formatexigens—I-52, CCUG 46960, Lactobacillus ruminis—E 194e, Clostridium bolteae—WAL 16351, [CCUG 46953], ATCC BAA-613, Song et al. 2003, Clostridium hiranonis—TO-931, JCM 10541, KCTC 15199, Clostridium scindens—VPI 13733, ATCC 35704, 19, Bacteroides xylanisolvens—XB1A, CCUG 53782, Clostridium sp.—L2-50, Clostridium orbiscindens—1_3_50AFAA, Alistipes shahii—WAL 8301, and Faecalibacterium prausnitzii—A2-165, JCM 31915.

In some embodiments, the defined gut microbial community comprises Acidaminococcus, Adlercreutzia, Akkermansia, Anaerostipes, Anaerotruncus, Bacteroides, Bifidobacterium, Bilophila, Blautia, Butyrivibrio, Clostridium, Collinsella, Coprococcus, Desulfovibrio, Eggerthella, Eubacterium, Faecalibacterium, Marvinbryantia, Mitsuokella, Odoribacter, Parabacteroides, Roseburia, Ruminococcus, Slackia, and Solobacterium. In certain embodiments, the defined gut microbial community comprises Acidaminococcus fermentans, Adlercreutzia equolifaciens, Akkermansia muciniphila, Anaerostipes caccae, Anaerotruncus colihominis, Bacteroides caccae, Bacteroides cellulosilyticus, Bacteroides dorei, Bacteroides eggerthii, Bacteroides fragilis, Bacteroides intestinalis, Bacteroides ovatus, Bacteroides pectinophilus, Bacteroides plebeius, Bacteroides stercoris, Bacteroides thetaiotaomicron, Bacteroides uniformis, Bacteroides vulgatus, Bifidobacterium breve, Bifidobacterium catenulatum, Bifidobacterium pseudocatenulatum, Bilophila wadsworthia, Blautia hansenii, Blautia hydrogenotrophica, Butyrivibrio crossotus, Clostridium asparagiforme, Clostridium hiranonis, Clostridium hylemonae, Clostridium leptum, Clostridium orbiscindens, Clostridium saccharolyticum, Clostridium scindens, Collinsella aerofaciens, Coprococcus comes, Desulfovibrio piger, Eggerthella lenta, Eubacterium rectale, Eubacterium siraeum, Eubacterium ventriosum, Faecalibacterium prausnitzii, Marvinbryantia formatexigens, Mitsuokella multacida, Odoribacter splanchnicus, Parabacteroides distasonis, Parabacteroides johnsonii, Parabacteroides merdae, Roseburia inulinivorans, Ruminococcus gnavus, Ruminococcus lactaris, Ruminococcus torques, Slackia exigua, and Solobacterium moorei.

In certain embodiments, the defined gut microbial community comprises Acidaminococcus fermentans—VR4, Acidaminococcus sp.—D21, Adlercreutzia equolifaciens—FJC-B9, Akkermansia muciniphila—Muc [CIP 107961], Alistipes finegoldii—AHN 2437, Alistipes indistinctus—JCM 16068, YIT 12060, Alistipes onderdonkii—WAL 8169, Anaerobutyricum hallii—VPI B4-27, Anaerofustis stercorihominis—ATCC BAA-858, CCUG 47767, CIP 108481, WAL 14563, Anaerostipes caccae—L1-92, Anaerotruncus colihominis —277, Bacteroides caccae—VPI 3452A [CIP 104201T, JCM 9498], Bacteroides cellulosilyticus—CRE21, CCUG 44979, Bacteroides coprocola—M16, Bacteroides coprophilus—CB42, JCM 13818, Bacteroides dorei —175, Bacteroides dorei —5_1_36/D4, Bacteroides eggerthii—ATCC 27754, NCTC 11155, Bacteroides finegoldii —199, Bacteroides fragilis—3_1_12, Bacteroides intestinalis —341, Bacteroides ovatus—NCTC 11153, Bacteroides rodentium—ST28, CCUG 59334, JCM 16469, Bacteroides thetaiotaomicron—1_1_6, Bacteroides fragilis—2_1_16, Bacteroides xylanisolvens—2_1_22, Parabacteroides distasonis—3_1_19, Bacteroides dorea—9_1_42FAA, Bacteroides ovatus—D2, Bacteroides stercoris—VPI B3-21, ATCC 43183, CIP 104203, JCM 9496, Bacteroides thetaiotaomicron—VPI 5482 [CIP 104206T, E50, NCTC 10582], Bacteroides uniformis—ATCC 8492, Bacteroides vulgatus—NCTC 11154, Bifidobacterium pseudocatenulatum—B1279, ATCC 27919, Bilophila wadsworthia—WAL 7959 [Lab 88-130H], Blautia hansenii—VPI C7-24, Blautia hydrogenotrophica—S5a33, Blautia obeum—ATCC 29174, KCTC 15206, VPI B3-21, Blautia sp.—KLE 1732, Blautia wexlerae—ATCC BAA-1564, JCM 17041, KCTC 5965, WAL 14507, Catenibacterium mitsuokai—RCA14-39, CIP 106738, JCM 10609, Clostridium asparagiforme—N6, CCUG 48471, Clostridium hylemonae—TN-271, JCM 10539, Clostridium leptum—VPI T7-24-1, ATCC 29065, Tyzzerella nexilis—DSM 1787, Clostridium saccharolyticum—WM1, ATCC 35040, NRC 2533, Absiella dolichum—DSM 3991, Collinsella aerofaciens—VPI 1003 [DSM 3979, JCM 10188], Collinsella stercoris—RCA 55-54, JCM 10641, Coprococcus comes—VPI CI-38, Dialister invisus—E7.25, CCUG 47026, Eubacterium rectale—VPI 0990 [CIP 105953], Eubacterium siraeum—VPI T9-50-2, ATCC 29066, DSM 3996, Eubacterium ventriosum—VPI 1013B, Coprococcus eutactus—VPI C33-22, Holdemanella biformis—VPI C17-5, ATCC 27806, KCTC 5969, Intestinibacter bartlettii—WAL 16138, ATCC BAA-827, CCUG 48940, Megasphaera sp.—Sanger 24, Sanger_24, Odoribacter splanchnicus—1651/6, ATCC 29572, CCUG 21054, CIP 104287, LMG 8202, NCTC 10825, Parabacteroides distasonis—NCTC 11152, Parabacteroides merdae—VPI T4-1, ATCC 43184, CCUG 38734, CIP 104202, JCM 9497, Parabacteroides sp.—D13, Granulicatella adiacens—GaD [CIP 103243, DSM 9848], Holdemania filiformis—VPI J1-31B-1, ATCC 51649, Hungatella hathewayi—1313, CCUG 43506, CIP 109440, MTCC 10951, Intestinimonas butyriciproducens—SRB-521-5-1, CCUG 63529, Solobacterium moorei—RCA59-74, CIP 106864, JCM 10645, Mitsuokella multacida—A 405-1, ATCC 27723, NCTC 10934, Olsenella uli—D76D-27C, ATCC 49627, CIP 109912, Parabacteroides johnsonii—M-165, CIP 109537, JCM 13406, Prevotella buccalis—HS4, ATCC 35310, NCDO 2354, Prevotella copri—CB7, JCM 13464, Roseburia inulinivorans—A2-194, CIP 109405, JCM 17584, NCIMB 14030, Clostridium sp.—VPI C48-50 (unassigned Clostridiales), Ruminococcus gauvreauii—CCRI-16110, CCUG 54292, JCM 14987, NML 060141, Ruminococcus lactaris—VPI X6-29, Ruminococcus torques—VPI B2-51, Alistipes putredinis—CCUG 45780, CIP 104286, ATCC 29800, Carlier 10203, VPI 3293, Alistipes senegalensis—CSUR P150, JCM 32779, JC50, Clostridium spiroforme—VPI C28-23-1A, ATCC 29900, NCTC 11211, Slackia exigua—S-7, ATCC 700122, JCM 11022, KCTC 5966, Bacteroides pectinophilus—N3, Butyrivibrio crossotus—T9-40A, ATCC 29175, Subdoligranulum variabile—BI-114, CCUG 47106, Turicibacter sanguinis—MOL361, NCCB 100008, Bifidobacterium breve—S1, ATCC 15700, NCTC 11815, Bifidobacterium catenulatum—B669, ATCC 27539, CECT 7362, CIP 104175, DSM 20103, Butyricimonas virosa—MT12, CCUG 56611, JCM 15149, Streptococcus salivarius subsp. thermophilus—LMD-9, Dorea formicigenerans—VPI C8-13 [JCM 9500], Bacteroides plebeius—M12, Ruminococcus gnavus—VPI C7-9, Oscillibacter sp.—KLE 1728, Clostridium sp.—M62/1, Slackia heliotrinireducens—RHS 1, ATCC 29202, NCTC 11029, Desulfovibrio piger—VPI C3-23 [DSM 749], Clostridium methylpentosum—R2, ATCC 43829, Ethanoligenens harbinense—UAN-3, CGMCC 1.5033, JCM 12961, Marvinbryantia formatexigens—I-52, CCUG 46960, Lactobacillus ruminis—E 194e, Clostridium bolteae—WAL 16351, [CCUG 46953], ATCC BAA-613, Song et al. 2003, Clostridium hiranonis—TO-931, JCM 10541, KCTC 15199, Clostridium scindens—VPI 13733, ATCC 35704, 19, Bacteroides xylanisolvens—XB1A, CCUG 53782, Clostridium sp.—L2-50, Clostridium orbiscindens—1_3_50AFAA, Alistipes shahii WAL 8301, and Faecalibacterium prausnitzii—A2-165, JCM 31915.

In some embodiments, community stability is characterized by up to 10% of the defined microbial strains dropping out following the microbial challenge. In some embodiments, community stability is characterized by the appearance of up to 10% of new strains contributed from the human fecal community appearing following the microbial challenge. In certain embodiments, at least 50% of the defined microbial strains are detectable following the microbial challenge. In certain embodiments, at least 60% of the defined microbial strains are detectable following the microbial challenge. In certain embodiments, at least 70% of the defined microbial strains are detectable following the microbial challenge. In certain embodiments, at least 80% of the defined microbial strains are detectable following the microbial challenge. In certain embodiments, at least 90% of the defined microbial strains are detectable following the microbial challenge. In certain embodiments, at least 95% of the defined microbial strains are detectable following the microbial challenge. In certain embodiments, at least 99% of the defined microbial strains are detectable following the microbial challenge.

In some embodiments, community stability is characterized by metagenomic analysis of a fecal sample obtained from the mouse following the microbial challenge. In certain embodiments, the metagenomic analysis is selected from whole genome sequencing, ribosomal gene sequencing, or ribosomal RNA sequencing. In certain embodiments, the whole genome sequencing is whole genome shotgun sequencing.

In some aspects of the above embodiments, the defined gut microbial community comprises between 100 and 200 defined microbial strains. In some embodiments, the defined gut microbial community comprises between 100 and 150 defined microbial strains.

In some aspects of the above embodiments, each defined microbial strain is molecularly identified. In some embodiments, the molecular identification comprises identification of a nucleic acid sequence that uniquely identifies each of the defined microbial strains. In certain embodiments, the nucleic acid sequence comprises a 16S rRNA sequence. In certain embodiments, the nucleic acid sequence comprises a whole genomic sequence. In certain embodiments, the molecular identification comprises Matrix-Assisted Laser

### Desorption/Ionization Time-of-Flight Mass Spectrometry.

In another aspect, provided herein is a method of treating an animal having a dysbiosis or pathological condition comprising administering a high-complexity defined gut microbial community according to any of the above embodiments. In certain embodiments, the animal is a mammal. In certain embodiments, the animal is a human. In certain embodiments, the high-complexity defined gut microbial community is administered via a route selected from the group consisting of oral, rectal, fecal (by enema), and naso/oro-gastric gavage.

In another aspect, provided herein is a method of making a high-complexity defined gut microbial community, wherein each of the plurality of defined microbial strains is individually cultured then combined to form the defined gut microbial community.

In another aspect, provided herein is a method of making a high-complexity defined gut microbial community, wherein all of the plurality of defined microbial strains are cultured together to form the defined gut microbial community.

In another aspect, provided herein is a method of making a high-complexity defined gut microbial community, wherein one or more of the plurality of defined microbial strains is individually cultured and two or more of the defined microbial strains are cultured together, and wherein the individually cultured defined microbial strains and the co-cultured defined microbial strains are combined together to form the defined gut microbial community.

In another aspect, provided herein is a formulation comprising the high-complexity defined gut microbial community and a pharmaceutically acceptable carrier or excipient.

### BRIEF DESCRIPTION OF THE DRAWINGS

FIG. 1 is a schematic illustrating a workflow to preparing a high-complexity defined gut microbial community.

FIG. 2 shows the relative abundance of microbial strains in mice colonized with a high-complexity defined microbial community and challenged with fecal samples prepared from 3 different human donors.

FIG. 3A shows a schematic of a treatment schedule of gnotobiotic mice colonized with human fecal samples, inoculated with C. difficile, and treated with a high-complexity defined gut microbial community. FIG. 3B shows a dot plot of C. difficile concentrations in the stool of mice treated in accordance with the treatment schedule of FIG. 3A.

FIG. 4 shows bar graphs of bile acid concentrations in stool (FIG. 4A) and cecum (FIG. 4B) from mice treated with human stool sample or high-complexity defined gut microbial community.

FIG. 5 shows bar graphs of metabolite concentrations in urine samples from mice treated with human stool sample or high-complexity defined gut microbial community.

### DETAILED DESCRIPTION

### 1. Definitions

The term “a” and “an” as used herein mean “one or more” and include the plural unless the context is appropriate.

As used herein, “abundance” of a specific gut microorganism refers to the number of individual organisms in an individual animal's gut. Abundance can be described as a proportion of the total gut population (e.g., number of organisms relative to the total gut population, the mass of the organism relative to the mass of the total gut population).

As used herein, “animal” refers to an organism to be treated with a microbial community, e.g., a high-complexity defined gut microbial community. Animals include, but are not limited to, mammals (e.g., murines, simians, equines, bovines, porcines, canines, felines, and the like), and more preferably include humans.

As used herein, “dysbiosis” refers to a state of a microbiome of the gut of an animal in which normal diversity and/or function is perturbed. In some instances, dysbiosis may be attributed to a decrease in the diversity of the gut microbiota, overabundance of one or more pathogens or pathobionts, or presence of pathogenic symbionts.

As used herein, the term “effective amount” refers to an amount sufficient to achieve a beneficial or desired result.

As used herein, a “humanized mouse” refers to a mouse with a human gut microbiome. A humanized mouse can be produced by removing the mouse's gut flora (e.g., by administering PEG-3350 and electrolytes, e.g., GoLYTELY® (Braintree Laboratories, Inc., Braintree, Mass.)) and/or administering broad spectrum antibiotics, and colonizing the mouse with a preparation of microorganisms from human feces. A humanized mouse can also refer to a gnotobiotic mouse that has been colonized with a human fecal sample. In some embodiments, the gut of the humanized mouse can be flushed (e.g., by administration of PEG-3350) before inoculation with a high-complexity gut microbial community described herein.

As used herein, an “isogenic gnotobiotic control mouse” refers to a mouse used as an experimental control that shares the same genotype as a mouse receiving administration of a microbial community, e.g., a high-complexity defined gut microbial community, but to which a vehicle control, or other experimental negative control, has been administered.

As used herein, the term “pharmaceutically acceptable carrier” refers to any of the standard pharmaceutical carriers, such as phosphate buffered saline (PBS) solution, water, emulsions (e.g., such as oil/water or water/oil emulsions), and various types of wetting agents. The compositions also can include stabilizers and preservatives. For examples of carriers, stabilizers, and adjuvants, see e.g., Martin, Remington's Pharmaceutical Sciences, 15^{th} Ed. Mack Publ. Co., Easton, Pa. [1975].

As used herein, “prevalence” of a gut microorganism refers to the frequency (e.g., number of individuals in a population) at which the organism is found in the human gut.

As used herein, “significantly” or “significant” refers to a change or alteration in a measurable parameter to a statistically significant degree as determined in accordance with an appropriate statistically relevant test. For example, in some embodiments, a change or alteration is significant if it is statistically significant in accordance with, e.g., a Student's t-test, chi-square, or Mann Whitney test.

As used herein, “minimal difference” refers to a change or alteration in a measurable parameter to a degree that is not statistically significant as determined in accordance with an appropriate statistically relevant test. For example, in some embodiments, a change or alteration is minimally different if it is not statistically significant in accordance with, e.g., a Student's t-test, chi-square, or Mann Whitney test.

As used herein, the term “metabolizing a substrate” means that a measurable reduction in the amount of the substrate can be demonstrated following contacting of the substrate with a microbial community, in vivo or in vitro, as compared to contacting with a vehicle control. In embodiments, the reduction in amount is determined using mass spectrometry. In embodiments, the contacting in vivo is achieved through introduction of the community into a gnotobiotic organism such as, e.g., a gnotobiotic mouse.

As used herein, the term “producing a metabolite” means that a measurable increase in the amount of the metabolite can be demonstrated following contacting one or more metabolite precursor molecules with a microbial community, in vivo or in vitro, as compared to contacting with a vehicle control. In embodiments, the increase in amount is determined using mass spectrometry. In embodiments, the contacting in vivo is achieved through introduction of the community into a gnotobiotic organism such as, e.g., a gnotobiotic mouse.

### 2. Fecal Microbiota Transplantation

Fecal microbiota transplantation (FMT) is remarkable in two ways that suggest its generality: 1) there has been a very low rate of acute adverse events, suggesting that this modality is likely to be generally safe; and 2) even though no concerted effort has been made to optimize the process of engraftment, it already works quite well for treating certain conditions. Taken together, these observations suggested to the inventors that, counterintuitively, one single community could in principle be transplanted stably into the gut of millions of patients and administration of a high-complexity defined gut microbial community may be safer and more predictable than seemingly simpler perturbations to the gut (e.g., addition or removal of one or a few strains). This is exciting, since administration of a high-complexity defined gut microbial community would be the biggest ‘lever’ one could pull in terms of controlling human biology linked to the microbiota. However, the current state of the art is fecal transplantation, which cannot be scaled. This calls for a new technology that enables the design and assembly of transplantable communities that are, on the one hand, completely defined, and on the other hand, approach the complexity of a native gut community.

### 3. Microbial Communities

As used herein, “community” or “microbial community” refers to a physical combination of a plurality of different microorganisms, usually a plurality of different bacterial strains, sometimes comprising one or more strains or archaea. A naturally occurring gut microbiome is one example of a community. An artificially created mixture of strains of known identity is another example of a community. A defined gut microbial community is yet another example of a community. As used herein, a “defined gut microbial community” means a combined plurality of microbial strains for engraftment in a gut of an animal wherein each microbial strain has been molecularly identified.

As used herein, a “microbial strain” refers to a type or sub-type of a microbe. As used herein, a “defined microbial strain” is a microbial strain that has been molecularly identified; e.g., a microbial strain whose whole genome has been sequenced. As used herein, a “plurality of defined microbial strains” means two or more microbial strains from two or more distinct microbial species. In some embodiments, multiple microbial strains in a plurality may represent a single microbial species.

As used herein, “complexity” means the number of strains in a community without regard to abundance. A community comprising 50 strains is more complex than a community comprising 15 strains. As used herein, “high-complexity” means a community having at least 40 defined microbial strains. In some embodiments, a high-complexity community comprises between 40 and 500, between 40 and 400, between 40 and 300, between 40 and 200, between 40 and 150, between 40 and 140, between 40 and 130, between 40 and 120, between 40 and 110, between 40 and 100, between 50 and 500, between 50 and 400, between 50 and 300, between 50 and 200, between 50 and 150, between 50 and 140, between 50 and 130, between 50 and 120, between 50 and 110, between 50 and 100, between 60 and 500, between 60 and 400, between 60 and 300, between 60 and 200, between 60 and 150, between 60 and 140, between 60 and 130, between 60 and 120, between 60 and 110, between 60 and 100, between 70 and 500, between 70 and 400, between 70 and 300, between 70 and 200, between 70 and 150, between 70 and 140, between 70 and 130, between 70 and 120, between 70 and 110, between 70 and 100, between 80 and 500, between 80 and 400, between 80 and 300, between 80 and 200, between 80 and 150, between 80 and 140, between 80 and 130, between 80 and 120, between 80 and 110, between 80 and 100, between 90 and 500, between 90 and 400, between 90 and 300, between 90 and 200, between 90 and 150, between 90 and 140, between 90 and 130, between 90 and 120, between 90 and 110, between 90 and 100, between 100 and 500, between 100 and 400, between 100 and 300, between 100 and 200, between 100 and 150, between 100 and 140, between 100 and 130, between 100 and 120, or between 100 and 110 defined microbial strains.

### 3.1. Culturing Microbial Strains and Communities

As used herein, “culture” (and grammatical variants thereof, e.g., “cultured,” and “culturing”) refers to the maintenance and/or growth of a microbial strain or microbial community in a liquid medium, or on a solid medium. For example, in some embodiments, culturing of purchased microbial strains is performed in accordance with the manufacturer's instructions.

As used herein, “aliquot,” refers to an in vitro bacterial population that is physically separated from other populations for storage, culture, analysis and the like. “Aliquot” may refer to separate populations in vessels, compartments, tubes, wells of multiwell plates, emulsion clonal, such as a stock of a strain isolate, or may be a mixture of strains, such as an artificial community or defined gut microbial community.

In certain embodiments, microbial strains or microbial communities are maintained or grown in specially formulated media such as the media described in any one of Tables 1-6 below.

TABLE 1
MEDIUM A

| | Amount | | Final |
|---|---|---|---|
| Component | (in 500 mL) | | Concentration |
| Trypticase ™ | 5 | g | 1% (w/v) |
| Peptone | | | |
| Yeast Extract | 2.5 | g | 0.5% (w/v) |
| D-(+)-Glucose | 1 | g | 0.2% (w/v) |
| L-Cysteine | 0.25 | g | 0.05% (w/v) |
| hydrochloride | | | |
| 1M Potassium | 50 | mL | 10% (v/v) |
| phosphate | | | |
| buffer, pH 7.2** | | | |
| TYG Salts | 20 | mL | 4% (w/v) |
| solution** | | | |
| Vitamin K | 500 | μL | 0.000001% (w/v) |
| solution** | of | | |
| | 1 | mg/mL | |
| 0.8% (w/v) | 500 | μL | |
| CaCl₂** | | | |
| FeSO₄ • 7 H₂O** | 500 | μL | |
| | of | | |
| | 0.4 | mg/mL | |
| Resazurin** | 2 | mL | 0.000001% (w/v) |
| | of | | |
| | 0.25 | mg/mL | |
| Histidine- | 500 | μl | |
| Hematin** | | | |
| D-(+)- | 0.5 | g | 0.1% (w/v) |
| Cellobiose | | | |
| D-(+)-Maltose | 0.5 | g | 0.1% (w/v) |
| monohydrate | | | |
| D-(−)-Fructose | 0.5 | g | 0.1% (w/v) |
| Soluble starch** | 12.5 | mL | 0.05% (w/v) |
| | of | | |
| | 2% | (w/v) | |
| Tween 80 | 1 | mL | 0.05% (v/v) |
| | of | | |
| | 25% | (v/v) | |
| Meat extract | 2.5 | g | 0.5% (w/v) |
| Trace Mineral | 5 | mL | 1% (v/v) |
| Supplement | | | |
| Vitamin | 5 | mL | 1% (v/v) |
| Supplement | | | |
| SCFA | 1.4 | mL | 0.28% (v/v) |
| supplement** | | | |
| Milli-Q water | 150 | mL | |
| (dH₂O)* | | | |

TABLE 2
MEDIUM B

| | Amount | | Final |
|---|---|---|---|
| Component | (in 1000 mL) | | Concentration |
| Lean Ground | 500 | g | 50% (w/v) |
| Beef (Fat Free) | | | |
| NaOH | 25 | mL | 2.5% (v/v) |
| Casitone | 30 | g | 3% (w/v) |
| Yeast Extract | 5 | g | 0.5% (w/v) |
| K₂HPO₄ | 5 | g | 0.5% (w/v) |
| Resazurin | 1 | mg | 0.01% (w/v) |
| (±) Haemin | 10.0 | mL | 1% (v/v) |
| Solution [1 mL | | | |
| IN NaOH in 100 | | | |
| mL of dH₂O] | | | |
| (±) Vitamin K₁ | 10.0 | mL | 1% (v/v) |
| Solution [0.1 mL | | | |
| Vitamin K₁ in 20 | | | |
| mL of 95% | | | |
| Ethanol] or | | | |
| Vitamin K₃ | | | |
| Solution [0.05 | | | |
| mg/mL Vitamin | | | |
| K₃ ub 95% | | | |
| Ethanol] | | | |
| NaHCO₃ | 1 | g | 0.1% (w/v) |
| Milli-Q water | To Final | | |
| (dH₂O)* | Volume of | | |
| | 1000 | mL | |

pH adjusted to 7.2

TABLE 3
MEDIUM C

| | Amount | | Final |
|---|---|---|---|
| Component | (in 1000 mL) | | Concentration |
| Trypticase | 5 | g | 0.5% (w/v) |
| Peptone | | | |
| Peptone | 5 | g | 0.5% (w/v) |
| Yeast Extract | 10 | g | 1% (w/v) |
| Beef Extract | 5 | g | 0.5% (w/v) |
| Glucose | 5 | g | 0.5% (w/v) |
| K₂HPO₄ | 2 | g | 0.2% (w/v) |
| Tween-80 | 1 | mL | 0.1% (v/v) |
| Cysteine-HCl × | 0.5 | g | 0.05% (w/v) |
| H₂O | | | |
| Resazurin | 1 | mg | 0.01% (w/v) |
| Salt Solution | 40 | mL | 4% (v/v) |
| [0.25 g CaCl₂ × | | | |
| 2 H₂O, 0.5 g | | | |
| MgSO₄ × 7 H₂O, | | | |
| 1 g K₂HPO₄, 1 g | | | |
| K₂HPO₄, 10 g | | | |
| NaHCO₃, 2 g | | | |
| NaCI, to 1000 | | | |
| mL in dH₂O] | | | |
| Haemin Solution | 10.0 | mL | 1% (v/v) |
| [1 mL 1N NaOH | | | |
| in 100 mL of | | | |
| dH₂O] | | | |
| Vitamin K₁ | 0.2 | mL | 0.02% (v/v) |
| Solution [0.1 mL | | | |
| Vitamin K₁ in 20 | | | |
| mL of 95% | | | |
| Ethanol] | | | |
| Milli-Q water | To Final | | |
| (dH₂O) | Volume of | | |
| | 1000 | mL | |

pH adjusted to 7.2 using 8N NaOH

TABLE 4
MEDIUM D

| | Amount | | Final |
|---|---|---|---|
| Component | (in 1000 mL) | | Concentration |
| Tryptose | 10 | g | 1% (w/v) |
| Beef Extract | 10 | g | 1% (w/v) |
| Yeast Extract | 3 | g | 0.3% (w/v) |
| Dextrose | 5 | g | 0.5% (w/v) |
| NaCl | 5 | g | 0.5% (w/v) |
| Soluble Starch | l | g | 0.1% (w/v) |
| L-Cysteine HCl | 0.5 | g | 0.05% (v/v) |
| Sodium Acetate | 3 | g | 0.3% (w/v) |
| Resazurin | 4 | mL | 0.0001% (w/v) |
| (0.025%) | | | |
| Milli-Q water | To Final | | |
| (dH₂O) | Volume of | | |
| | 1000 | mL | |

pH adjusted to 6.8

TABLE 5
MEDIUM F

| | Amount | | Final |
|---|---|---|---|
| Component | (in 1000 mL) | | Concentration |
| Casein peptone, | 10 | g | 1% (w/v) |
| tryptic digest | | | |
| Yeast Extract | 5 | g | 0.5% (w/v) |
| Meat Extract | 5 | g | 0.5% (w/v) |
| Bacto Soy tone | 5 | g | 0.5% (w/v) |
| Glucose | 10 | g | 1% (w/v) |
| K₂HPO₄ | 2 | g | 0.2% (w/v) |
| MgSO₄ × 7 H₂O | 0.2 | g | 0.02% (w/v) |
| MnSO₄ × H₂O | 0.05 | g | 0.005% (w/v) |
| Tween 80 | 1 | ml | 0.1% (v/v) |
| NaCl | 5 | g | 0.5% (w/v) |
| Cysteine-HCl × | 0.5 | g | 0.05% (w/v) |
| H₂O | | | |
| Salt Solution | 40 | mL | 4% (v/v) |
| [0.25 g CaCl₂ × | | | |
| 2H₂O, 0.5 g | | | |
| MgSO₄ × 7 H₂O, | | | |
| 1 g K₂HPO₄, 1 g | | | |
| K₂HPO₄, 10 g | | | |
| NaHCO₃, 2 g | | | |
| NaCl, to 1000 | | | |
| mL in dH₂O] | | | |
| Resazurin (25 | 4 | mL | 0.01% (w/v) |
| mg/100 mL) | | | |
| Milli-Q water | To Final | | |
| (dH₂O) | Volume of | | |
| | 1000 | mL | |

pH adjusted to 6.8 using 8N NaOH

TABLE 6
YCFAC Broth

| | Amount | | Final |
|---|---|---|---|
| Component | (in 1000 mL) | | Concentration |
| Casitone | 10 | g | 1% (w/v) |
| Yeast Extract | 2.5 | g | 0.25% (w/v) |
| Sodium | 4 | g | 0.4% (w/v) |
| Bicarbonate | | | |
| Glucose | 2 | g | 0.2% (w/v) |
| Cellobiose | 2 | g | 0.2% (w/v) |
| Maltose | 2 | g | 0.2% (w/v) |
| Potassium | 0.45 | g | 0.045% (w/v) |
| Phosphate | | | |
| Monobasic | | | |
| Potassium | 0.45 | g | 0.045% (w/v) |
| Phosphate | | | |
| Dibasic | | | |
| Sodium Chloride | 0.9 | g | 0.09% (w/v) |
| Ammonium | 0.9 | g | 0.09% (w/v) |
| Sulfate | | | |
| Magnesium | 0.09 | g | 0.009% (w/v) |
| Sulfate | | | |
| Heptahydrate | | | |
| Calcium | 0.09 | g | 0.009% (w/v) |
| chloride | | | |
| Hemin (0.1% | 10 | mL | 0.001% (w/v) |
| solution) | | | |
| Vitamin | 10 | mL | 1% (v/v) |
| Supplement | | | |
| [Folic acid 2.0 | | | |
| mg/1, Pyridoxine | | | |
| hydrochloride | | | |
| 10.0 mg/L, | | | |
| Riboflavin 5.0 | | | |
| mg/L, Biotin 2.0 | | | |
| mg/L. Thiamine | | | |
| 5.0 mg/L, | | | |
| Nicotinic acid | | | |
| 5.0 mg/L, | | | |
| Calcium | | | |
| Pantothenate 5.0 | | | |
| mg/liter, | | | |
| Vitamin B12 0.1 | | | |
| mg/L, p- | | | |
| Aminobenzoic | | | |
| acid 5.0 mg/L, | | | |
| Thioctic acid 5.0 | | | |
| mg/L, | | | |
| Monopotassium | | | |
| phosphate 900.0 | | | |
| mg/L] | | | |
| Resazurin | 4 | mL | 0.0001% (w/v) |
| (0.025% | | | |
| solution) | | | |
| L-Cysteine (25% | 4 | mL | 0.01% (w/v) |
| solution) | | | |
| Volatile Fatty | 2.9 | mL | 0.29% (v/v) |
| Acid Solution | | | |
| Milli-Q water | To Final | | |
| (dH₂O) | Volume of | | |
| | 1000 | mL | |

pH adjusted to 6.8

### 3.2. Engraftment

As used herein, “engraftment” (and grammatical variants thereof, e.g., “engraft”) refers to the ability of a microbial strain or microbial community to establish in one or more niches of the gut of an animal. Operationally, a microbial strain or microbial community is “engrafted” if evidence of its establishment, post-administration, can be obtained. In some embodiments, that evidence is obtained by molecular identification (e.g., Matrix-Assisted Laser Desorption/Ionization Time-Of-Flight Mass Spectrometry (MALDI-TOF MS), liquid chromatography-mass spectrometry (LC-MS), 16S rRNA sequencing, or genomic sequencing) of a sample obtained from the animal. In some embodiments, the sample is a stool sample. In some embodiments, the sample is a biopsy sample taken from the gut of the animal (e.g., from a location along the gastrointestinal tract of the animal). Engraftment may be transient or may be persistent. In some embodiments, transient engraftment means that the microbial strain or microbial community can no longer be detected in an animal to which it has been administered after the lapse of about 1 week, about 2 weeks, about three weeks, about 1 month, about 2 months, about 3 months, about 4 months, about 6 months, about 8 month, about 10 months, about 1 year, about 1.5 years, or about 2 years.

As used herein, “substantial engraftment” refers to that at a defined timepoint following administration to an animal (e.g., in some embodiments, a gnotobiotic mouse) of the microbial community (e.g., a high-complexity defined gut microbial community), evidence of the engraftment of at least 70% of the administered defined microbial strains can be demonstrated. For example, in some embodiments, substantial engraftment is achieved when at least 72%, at least 74%, at least 76%, at least 78%, at least 80%, at least 82%, at least 84%, at least 86%, at least 88%, at least 90%, at least 92%, at least 94%, at least 96%, at least 98%, or 100% of the administered defined microbial strains can be demonstrated. In some embodiments, such evidence is obtained by metagenomic analysis of a stool sample obtained from the animal. In some embodiments, “substantial engraftment” is achieved when an intended metabolic phenotype is demonstrably present in the recipient post-administration. In some embodiments, the defined timepoint is between 1 week and 52 weeks. For example, in some embodiments, the defined timepoint is between 1 week and 48 weeks, 1 week and 42 weeks, 1 week and 36 weeks, 1 week and 30 weeks, 1 week and 24 week, 1 week and 18 weeks, 1 week and 12 weeks, 1 week and 10 weeks, 1 week and 8 weeks, 1 week and 6 weeks, 1 week and 4 weeks, 1 week and 2 weeks, 2 weeks and 52 weeks, 2 weeks and 48 weeks, 2 weeks and 36 weeks, 2 weeks and 30 weeks, 2 ad 24 weeks, 2 weeks and 18 weeks, 2 weeks and 12 weeks, 2 weeks and 10 weeks, 2 weeks and 8 weeks, 2 weeks and 6 weeks, 2 weeks and 4 weeks, 4 weeks and 52 weeks, 4 weeks and 48 weeks, 4 weeks and 42 weeks, 4 weeks and 36 weeks, 4 weeks and 30 weeks, 4 weeks and 24 weeks, 4 weeks and 18 weeks, 4 weeks and 12 weeks, 4 weeks and 10 weeks, 4 weeks and 8 weeks, 4 weeks and 6 weeks, 6 weeks and 52 weeks, 6 weeks and 48 weeks, 6 weeks and 42 weeks, 6 weeks and 36 weeks, 6 weeks and 30 weeks, 6 weeks and 24 weeks, 6 weeks and 18 weeks, 6 weeks and 12 weeks, 6 weeks and 10 weeks, 6 weeks and 8 weeks, 8 weeks and 52 weeks, 8 weeks and 48 weeks, 8 weeks and 42 weeks, 8 weeks and 36 weeks, 8 weeks and 30 weeks, 8 weeks and 24 weeks, 8 weeks and 18 weeks, 8 weeks and 12 weeks, or 8 weeks and 10 weeks.

### 3.3. Stability

As used herein, “human fecal community microbial challenge” refers to administration of a human stool sample into the gut of an animal that has previously been colonized with a microbial community, e.g., a high-complexity defined gut microbial community.

In some embodiments, stability of a community refers to the ability of defined microbial strains comprising a community to persist (i.e. remain engrafted) in a gut of an animal following microbial challenge. In some embodiments, when given sufficient time to permit colonization of microbial challenge strains in the gut of an animal engrafted with a high-complexity defined gut microbial community, a stable community can be defined as one where at least 50%, at least 60%, at least 70%, at least 80%, at least 90%, at least 95%, at least 96%, at least 97%, at least 98%, or at least 99% of the defined microbial strains are detectable by metagenomic analysis. For example, in some embodiments, metagenomic analysis comprises whole genome shotgun sequencing analysis.

In some embodiments, stability can be demonstrated at a time range of between at least 1 week and 52 weeks. For example, in some embodiments, stability can be demonstrated at a time rage of between at least 1 week and 48 weeks, 1 week and 42 weeks, 1 week and 36 weeks, 1 week and 30 weeks, 1 week and 24 week, 1 week and 18 weeks, 1 week and 12 weeks, 1 week and 10 weeks, 1 week and 8 weeks, 1 week and 6 weeks, 1 week and 4 weeks, 1 week and 2 weeks, 2 weeks and 52 weeks, 2 weeks and 48 weeks, 2 weeks and 36 weeks, 2 weeks and 30 weeks, 2 ad 24 weeks, 2 weeks and 18 weeks, 2 weeks and 12 weeks, 2 weeks and 10 weeks, 2 weeks and 8 weeks, 2 weeks and 6 weeks, 2 weeks and 4 weeks, 4 weeks and 52 weeks, 4 weeks and 48 weeks, 4 weeks and 42 weeks, 4 weeks and 36 weeks, 4 weeks and 30 weeks, 4 weeks and 24 weeks, 4 weeks and 18 weeks, 4 weeks and 12 weeks, 4 weeks and 10 weeks, 4 weeks and 8 weeks, 4 weeks and 6 weeks, 6 weeks and 52 weeks, 6 weeks and 48 weeks, 6 weeks and 42 weeks, 6 weeks and 36 weeks, 6 weeks and 30 weeks, 6 weeks and 24 weeks, 6 weeks and 18 weeks, 6 weeks and 12 weeks, 6 weeks and 10 weeks, 6 weeks and 8 weeks, 8 weeks and 52 weeks, 8 weeks and 48 weeks, 8 weeks and 42 weeks, 8 weeks and 36 weeks, 8 weeks and 30 weeks, 8 weeks and 24 weeks, 8 weeks and 18 weeks, 8 weeks and 12 weeks, or 8 weeks and 10 weeks.

In other embodiments, stability of a community refers to the characteristic of defined microbial strains comprising a community to maintain a metabolic phenotype over a period of time or following microbial challenge. For example, in some embodiments, defined microbial strains comprising a community can maintain a metabolic phenotype for at least 1 week, at least 2 weeks, at least 3 weeks, at least 4 weeks, at least 6 weeks, at least 8 weeks, at least 10 weeks, at least 12 weeks, at least 4 months, at least 6 months at least 8 months, at least 10 months, at least 1 year, at least 1.5 years, or at least 2 years.

In some embodiments, a stable community can be defined as one where the defined microbial strains comprising the community maintain the ability to metabolize at least 90% (e.g., at least 91%, at least 92%, at least 93%, at least 94%, at least 95%, at least 96%, at least 97%, at least 98%, at least 99%, or all) of substrates selected from the group consisting of: a-mannan (yeast), acetate, agarose, alanine, alginate, anthocyanin, arabinan, arabinogalactan, arabinoxylan, arginine, asparagine, Aspartate, b-glucans, butyrate, carrageenan, chitin, chlorogenic acids, chondroitin sulfate, cinnamic acid, Cysteine, dextran (40), Dihydrochalcones, Enterodiol, flavan-3-ols, flavanones, flavones, flavonols, folate, formate, galactomannan (carob), galacturonan (homo), galacturonate, glucomannan (konjac), glutamate, Glutamine, Glycine, Histidine, hyaluronan, hydrogen, hydroxycinnamic acids, hydroxyproline, inulin, isoflavones/isoflavanones, Isoleucine, lactate, laminarin, Leucine, levan, Lysine, Methionine, mucin O-linked glycans, Ornithine, Phenylalanine, porphyran, Proline, propionate, rhamnogalacturonan I, rhamnogalacturonan II, Secoisolariciresinol diglucoside, Serine, starch (potato), starch (structure 1), thiamine, Threonine, tryptophan, Tyrosine, Valine, xyloglucan, and xylooligosaccharides (XOS), over a period of time or following microbial challenge.

In some embodiments, a stable community can be defined as one where the defined microbial strains comprising the community maintain the ability to produce at least 90% (e.g., at least 91%, at least 92%, at least 93%, at least 94%, at least 95%, at least 96%, at least 97%, at least 98%, at least 99%, or all) of metabolites selected from the group consisting of: formate, acetate, propionate, butyrate, isobutyrate, valerate, isovalerate, 2-methylbutyrate, caporate, isocaporate, 3-methylvaleric acid, L-phenylalanine, 3-phenylpropionic acid, phyenypyruvate, DL-3-phenyllactic acid, trans-cinnamic acid, phenyllactic acid, phenethylamine, L-tyrosine, 3-(4-hydroxyphenyl)propionic acid, 3-(4-hydroxyphenyl) pyruvic acid, DL-p-hydroxyphenyl lactic acid, p-coumaric acid, 4-hydroxyphenyl acetic acid, tyramine, phenol, p-cresol, 4-ethylphenol, 4-vinylphenol, 4-hydroxybenzoic acid, L-tryptophan, 3-indolepropionic acid, 3-indolepyruvic acid, DL-indole-3-lactic acid, trans-3-indoleacrylic acid, 3-indoleacetic acid, tryptamine, indole, skatol, indole-3-carboxylic acid, indole-3-carboxyaldehyde, N-acetyl-L-phenylalanine, phenylpropionylglycine, 3-(3-hydroxyphenyl) propionic acid, cinnamoylglycine, phenylacetylglycine, phenylacetylglutamine, hippuric acid, 2-hydroxyhippuric acid, 3-hydroxyhippuric acid, 4-hydroxyhippuric acid, 4-hydroxyphenylacetylglycine, phenyl sulfate, phenyl glucuronide, p-cresol sulfate, p-cresol glucuronide, 4-ethylphenol sulfate, 4-ethylphenol glucuronide, N-acetyl-L-tryptophan, 5-hydroxy-L-typtophan, N-acetyl serotonin, 3-indolepriopionylglycine, indolyl-3-acryloylglycine, indoxyl sulfate, indoxyl glucuronide, 5-hydroxyindole-3-acetic acid, indoleacetylglycine, lithocholic acid, murocholic acid, ursodeoxycholic acid, hyodeoxycholic acid, deoxycholic acid, 7-oxocholic acid, ω-muricholic acid, α-muricholic acid, β-muricholic acid, γ-muricholic acid, 7βcholic acid, taurolithocholic acid, tauroursodeoxycholic acid, taurohyodeoxycholic acid, taurochenodeoxycholic acid, taurodeoxycholic acid, tauro-β-muricholic acid, tauro-ω-muricholic acid, and taurocholic acid, over a period of time or following microbial challenge.

In some embodiments, a stable community can be defined as one where the ability to utilize at least 90% (e.g., at least 91%, at least 92%, at least 93%, at least 94%, at least 95%, at least 96%, at least 97%, at least 98%, at least 99%, or all) of the MetaCyc metabolic pathways selected from the group consisting of: 1CMET2-PWY, 2.6.1.32-RXN, AEROBACTINSYN-PWY, ALACAT2-PWY, ALADEG-PWY, ALANINE-DEG3-PWY, ALANINE-SYN2-PWY, ALANINE-VALINESYN-PWY, ANAPHENOXI-PWY, ARGASEDEG-PWY, ARGDEG-III-PWY, ARGDEG-IV-PWY, ARGDEGRAD-PWY, ARGDEG-V-PWY, ARG-GLU-PWY, ARGININE-SYN4-PWY, ARG-PRO-PWY, ARGSYNBSUB-PWY, ARGSYN-PWY, ASPARAGINE-BIOSYNTHESIS, ASPARAGINE-DEG1-PWY, ASPARAGINE-DEG1-PWY-1, ASPARAGINESYN-PWY, ASPARTATE-DEG1-PWY, ASPARTATESYN-PWY, ASPASN-PWY, ASPSYNII-PWY, AST-PWY, BETA-ALA-DEGRADATION-I-PWY, CAMALEXIN-SYN, CITRULBIO-PWY, CITRULLINE-DEG-PWY, COA-PWY, CODH-PWY, CYSTEINE-DEG-PWY, CYSTSYN-PWY, DAPLYSINESYN-PWY, ENTBACSYN-PWY, ETHYL-PWY, FAO-PWY, FERMENTATION-PWY, GLNSYN-PWY, GLUDEG-I-PWY, GLUGLNSYN-PWY, GLUTAMATE-DEG1-PWY, GLUTAMATE-SYN2-PWY, GLUTAMINDEG-PWY, GLUTAMINEFUM-PWY, GLUTATHIONESYN-PWY, GLUTDEG-PWY, GLUTORN-PWY, GLUTSYNIII-PWY, GLUTSYN-PWY, GLYCGREAT-PWY, GLYSYN-ALA-PWY, GLYSYN-PWY, GLYSYN-THR-PWY, HISDEG-PWY, HISHP-PWY, HISTDEG-PWY, HISTSYN-PWY, HOMOCYSDEGR-PWY, HOMOSER-METSYN-PWY, HOMOSERSYN-PWY, HSERMETANA-PWY, HYDROXYPRODEG-PWY, ILEUDEG-PWY, ILEUSYN-PWY, LARABITOLUTIL-PWY, LCYSDEG-PWY, LEU-DEG2-PWY, LEUSYN-PWY, LYSDEGII-PWY, LYSINE-AMINOAD-PWY, LYSINE-DEG1-PWY, MALATE-ASPARTATE-SHUTTLE-PWY, METH-ACETATE-PWY, METHANOGENESIS-PWY, METHIONINE-DEG1-PWY, MGLDLCTANA-PWY, ORN-AMINOPENTANOATE-CAT-PWY, ORNDEG-PWY, P101-PWY, P162-PWY, P163-PWY, P181-PWY, P261-PWY, P283-PWY, P401-PWY, P541-PWY, PHENYLALANINE-DEG1-PWY, PHESYN, PHOSLIPSYN2-PWY, PHOSPHONOTASE-PWY, PROSYN-PWY, PROUT-PWY, PWY0-1021, PWY0-1221, PWY0-1299, PWY0-1303, PWY0-1305, PWY0-1313, PWY0-1317, PWY0-1321, PWY0-1338, PWY0-1347, PWY0-1355, PWY0-1356, PWY0-1534, PWY0-1544, PWY0-1565, PWY0-1576, PWY0-1577, PWY0-1578, PWY0-1585, PWY0-1601, PWY0-42, PWY0-461, PWY0-823, PWY0-901, PWY-1, PWY-1061, PWY-1121, PWY-1186, PWY1-2, PWY-1263, PWY-1622, PWY-1722, PWY-1781, PWY-181, PWY-1881, PWY-1962, PWY-1981, PWY1F-467, PWY1F-FLAVSYN, PWY1G-0, PWY-2021, PWY-2161, PWY-2181, PWY-2201, PWY-2821, PWY-2941, PWY-2942, PWY-3, PWY-3022, PWY-3081, PWY-3161, PWY-3162, PWY-3181, PWY-3301, PWY-3341, PWY-3385, PWY-3461, PWY-3462, PWY-3581, PWY-361, PWY-3661, PWY-3661-1, PWY-381, PWY-3841, PWY-3941, PWY3DJ-12, PWY3O-4107, PWY3O-4108, PWY-4, PWY-40, PWY-4002, PWY-4041, PWY-4201, PWY-4281, PWY-43, PWY-4321, PWY-4341, PWY-4361, PWY-46, PWY490-3, PWY490-4, PWY-4981, PWY-4983, PWY-4984, PWY4FS-6, PWY-5, PWY-5022, PWY-5024, PWY-5028, PWY-5029, PWY-5030, PWY-5031, PWY-5041, PWY-5048, PWY-5049, PWY-5057, PWY-5075, PWY-5076, PWY-5078, PWY-5079, PWY-5081, PWY-5082, PWY-5087, PWY-5097, PWY-5101, PWY-5103, PWY-5104, PWY-5108, PWY-5109, PWY-5129, PWY-5135, PWY-5136, PWY-5151, PWY-5154, PWY-5155, PWY-5159, PWY-5176, PWY-5188, PWY-5189, PWY-5196, PWY-5199, PWY-5207, PWY-5250, PWY-5254, PWY-5265, PWY-5280, PWY-5283, PWY-5290, PWY-5297, PWY-5298, PWY-5311, PWY-5314, PWY-5316, PWY-5319, PWY-5324, PWY-5329, PWY-5331, PWY-5332, PWY-5364, PWY-5381, PWY-5382, PWY-5386, PWY-5394, PWY-5399, PWY-5436, PWY-5437, PWY-5441, PWY-5443, PWY-5458, PWY-5467, PWY-5468, PWY-5473, PWY-5474, PWY-5494, PWY-5497, PWY-5499, PWY-5629, PWY-5651, PWY-5653, PWY-5665, PWY-5669, PWY-5675, PWY-5679, PWY-5686, PWY-5710, PWY-5736, PWY-5737, PWY-5739, PWY-5740, PWY-5742, PWY-5747, PWY-5748, PWY-5751, PWY-5754, PWY-5766, PWY-5770, PWY-5784, PWY-5788, PWY-5797, PWY-5800, PWY-581, PWY-5811, PWY-5818, PWY-5826, PWY-5877, PWY-5883, PWY-5886, PWY-5912, PWY-5913, PWY-5921, PWY-5936, PWY-5940, PWY-5958, PWY-5963, PWY-5968, PWY-5978, PWY-5980, PWY-5990, PWY-6003, PWY-6004, PWY-601, PWY-6030, PWY-6039, PWY-6045, PWY-6052, PWY-6053, PWY-6054, PWY-6055, PWY-6068, PWY-6069, PWY-6082, PWY-6120, PWY-6121, PWY-6122, PWY-6123, PWY-6124, PWY-6133, PWY-6134, PWY-6141, PWY-6143, PWY-6148, PWY-6151, PWY-6160, PWY-6173, PWY-6196, PWY-6219, PWY-622, PWY-6220, PWY-6233, PWY-6273, PWY-6277, PWY-6281, PWY-6307, PWY-6309, PWY-6313, PWY-6318, PWY-6320, PWY-6321, PWY-6322, PWY-6324, PWY-6328, PWY-6334, PWY-6339, PWY-6343, PWY-6344, PWY-6345, PWY-6346, PWY-6375, PWY-6376, PWY-6381, PWY-6386, PWY-6387, PWY-6397, PWY-6403, PWY-6407, PWY-6408, PWY-6409, PWY-6431, PWY-6435, PWY-6444, PWY-6455, PWY-6456, PWY-6457, PWY-6466, PWY-6471, PWY-6473, PWY-6481, PWY-6486, PWY-6493, PWY-6495, PWY-6511, PWY-6512, PWY-6519, PWY-6533, PWY-6535, PWY-6536, PWY-6537, PWY-6543, PWY-6549, PWY-6559, PWY-6562, PWY-6572, PWY-6573, PWY-6574, PWY-6578, PWY-6588, PWY-6614, PWY-6627, PWY66-301, PWY66-375, PWY-6638, PWY66-391, PWY-6642, PWY66-420, PWY66-421, PWY66-425, PWY66-426, PWY66-428, PWY-6643, PWY-6661, PWY-6673, PWY-6682, PWY-6690, PWY-6696, PWY-6711, PWY-6717, PWY-6720, PWY-6724, PWY-6728, PWY-6731, PWY-6735, PWY-6749, PWY-6769, PWY-6771, PWY-6772, PWY-6773, PWY-6781, PWY-6784, PWY-6790, PWY-6791, PWY-6802, PWY-6807, PWY-6808, PWY-6813, PWY-6815, PWY-6816, PWY-6817, PWY-6818, PWY-6821, PWY-6822, PWY-6823, PWY-6831, PWY-6832, PWY-6834, PWY-6840, PWY-6845, PWY-6853, PWY-6854, PWY-6855, PWY-6891, PWY-6892, PWY-6896, PWY-6898, PWY-6902, PWY-6907, PWY-6908, PWY-6920, PWY-6922, PWY-6936, PWY-6942, PWY-6949, PWY-6953, PWY-6955, PWY-6963, PWY-6964, PWY-6965, PWY-6968, PWY-6969, PWY-6981, PWY-6982, PWY-6986, PWY-6994, PWY-7000, PWY-701, PWY-7014, PWY-7015, PWY-7016, PWY-7018, PWY-7019, PWY-702, PWY-7022, PWY-7025, PWY-7028, PWY-7040, PWY-7046, PWY-7052, PWY-7054, PWY-7064, PWY-7072, PWY-7088, PWY-7090, PWY-7097, PWY-7104, PWY-7115, PWY-7117, PWY-7118, PWY-7126, PWY-7147, PWY-7153, PWY-7158, PWY-7176, PWY-7177, PWY-7185, PWY-7186, PWY-7219, PWY-7221, PWY-7234, PWY-7246, PWY-7248, PWY-7250, PWY-7255, PWY-7274, PWY-7275, PWY-7282, PWY-7288, PWY-7297, PWY-7304, PWY-7315, PWY-7316, PWY-7318, PWY-7342, PWY-7351, PWY-7356, PWY-7376, PWY-7377, PWY-7383, PWY-7387, PWY-7397, PWY-7398, PWY-7400, PWY-7414, PWY-7425, PWY-7430, PWY-7432, PWY-7440, PWY-7441, PWY-7456, PWY-7467, PWY-7498, PWY-7501, PWY-7506, PWY-7510, PWY-7514, PWY-7518, PWY-7520, PWY-7525, PWY-7531, PWY-7532, PWY-7533, PWY-7536, PWY-7542, PWY-7543, PWY-7547, PWY-7549, PWY-7550, PWY-7555, PWY-7561, PWY-7565, PWY-7570, PWY-7571, PWY-7600, PWY-7605, PWY-761, PWY-7612, PWY-7626, PWY-7645, PWY-7648, PWY-7649, PWY-7650, PWY-7665, PWY-7667, PWY-7668, PWY-7669, PWY-7671, PWY-7674, PWY-7688, PWY-7690, PWY-7693, PWY-7694, PWY-7701, PWY-7704, PWY-7706, PWY-7708, PWY-7717, PWY-7718, PWY-7719, PWY-7733, PWY-7734, PWY-7735, PWY-7737, PWY-7751, PWY-7761, PWY-7765, PWY-7767, PWY-7769, PWY-7770, PWY-7782, PWY-7790, PWY-7791, PWY-7793, PWY-7797, PWY-7811, PWY-7814, PWY-7822, PWY-7824, PWY-7826, PWY-7842, PWY-7850, PWY-7851, PWY-7855, PWY-7860, PWY-7861, PWY-7863, PWY-7867, PWY-7870, PWY-7880, PWY-7888, PWY-7889, PWY-7891, PWY-7892, PWY-7897, PWY-7901, PWY-7904, PWY-7907, PWY-7909, PWY-7910, PWY-7913, PWY-7917, PWY-7930, PWY-7931, PWY-7936, PWY-7953, PWY-7955, PWY-7956, PWY-7957, PWY-7958, PWY-7959, PWY-7960, PWY-7962, PWY-7977, PWY-7985, PWY-7986, PWY-7987, PWY-7988, PWY-7990, PWY-8002, PWY-8003, PWY-8006, PWY-8007, PWY-8008, PWY-8009, PWY-801, PWY-8010, PWY-8011, PWY-8013, PWY-8014, PWY-8015, PWY-8016, PWY-8017, PWY-8024, PWY-8032, PWY-8040, PWY-8043, PWY-8045, PWY-8071, PWY-8072, PWY-8080, PWY-8081, PWY-8082, PWY-8083, PWY-8086, PWY-8088, PWY-842, PWY-861, PWY-862, PWY8J2-1, PWY8J2-22, PWYDQC-4, PWYG-321, PWY-I9, PWYQT-4450, PWYQT-4476, PYRIDNUCSAL-PWY, PYRIDNUCSYN-PWY, PYRIDOXSYN-PWY, SAM-PWY, SERDEG-PWY, SERSYN-PWY, SPHINGOLIPID-SYN-PWY, TAURINEDEG-PWY, THRDLCTCAT-PWY, THREONINE-DEG2-PWY, TRNA-CHARGING-PWY, TRPCAT-PWY, TRPIAACAT-PWY, TRPKYNCAT-PWY, TRPSYN-PWY, TRYPDEG-PWY, TYRFUMCAT-PWY, TYRSYN, UDPNACETYLGALSYN-PWY, UDPNAGSYN-PWY, VALDEG-PWY, and VALSYN-PWY, is maintained over a period of time or following microbial challenge.

In some embodiments, a stable community can be defined as one maintaining the ability to metabolize at least 90% (e.g., at least 91%, at least 92%, at least 93%, at least 94%, at least 95%, at least 96%, at least 97%, at least 98%, at least 99%, or all) of the substrates described above and produce at least 90% (e.g., at least 91%, at least 92%, at least 93%, at least 94%, at least 95%, at least 96%, at least 97%, at least 98%, at least 99%, or all) of the metabolites described above over a period of time or following microbial challenge.

In some embodiments, a stable community can be defined as one maintaining the ability to metabolize at least 90% (e.g., at least 91%, at least 92%, at least 93%, at least 94%, at least 95%, at least 96%, at least 97%, at least 98%, at least 99%, or all) of the substrates described above and utilize at least 90% (e.g., at least 91%, at least 92%, at least 93%, at least 94%, at least 95%, at least 96%, at least 97%, at least 98%, at least 99%, or all) of the MetaCyc metabolic pathways described above over a period of time or following microbial challenge.

In some embodiments, a stable community can be defined as one maintaining the ability to produce at least 90% (e.g., at least 91%, at least 92%, at least 93%, at least 94%, at least 95%, at least 96%, at least 97%, at least 98%, at least 99%, or all) of the metabolites described above and utilize at least 90% (e.g., at least 91%, at least 92%, at least 93%, at least 94%, at least 95%, at least 96%, at least 97%, at least 98%, at least 99%, or all) of the MetaCyc metabolic pathways described above over a period of time or following microbial challenge.

In some embodiments, a stable community can be defined as one maintaining the ability to metabolize at least 90% (e.g., at least 91%, at least 92%, at least 93%, at least 94%, at least 95%, at least 96%, at least 97%, at least 98%, at least 99%, or all) of the substrates described above, produce at least 90% (e.g., at least 91%, at least 92%, at least 93%, at least 94%, at least 95%, at least 96%, at least 97%, at least 98%, at least 99%, or all) of the metabolites described above, and utilize at least 90% (e.g., at least 91%, at least 92%, at least 93%, at least 94%, at least 95%, at least 96%, at least 97%, at least 98%, at least 99%, or all) of the MetaCyc metabolic pathways described above over a period of time or following microbial challenge.

As used herein, “dropping out” refers to an event where a microbial strain in a microbial community does not stably engraft following administration into the gut of an animal. For example, in some embodiments, a microbial community is stable if up to 10% of the defined microbial strains drop out following microbial challenge. In some embodiments, a microbial community is stable if up to 9%, up to 8%, up to 7%, up to 6%, up to 5%, up to 4%, up to 3%, up to 2%, or up to 1% of the defined microbial strains drop out following microbial challenge.

As used herein, “jumping in” refers to an event where a microbial strain that is not present in a microbial community at the time of being administered into an animal, stably engrafts into one or more niche in the gut of the animal and becomes part of the engrafted microbial community. In some embodiments, a microbial strain that jumps in originates from an animal's gut commensal repertoire, a fecal community microbial challenge, or from an administration into the gut of an animal subsequent to an initial administration of the microbial community. For example, in some embodiments, a microbial community is stable if up to 10% of new strains are contributed by a microbial challenge (e.g., a human fecal community microbial challenge). In some embodiments, a microbial community is stable if up to 9%, up to 8%, up to 7%, up to 6%, up to 5%, up to 4%, up to 3%, up to 2%, or up to 1% of new strains are contributed by a microbial challenge.

### 4. Metagenomic Analysis and Molecular Identification

As used herein, “metagenomic analysis” refers to use of massively parallel sequencing for analyzing a microbiome, or defined gut microbial community. As used herein, metagenomic analysis includes, without limitation, whole genome sequencing (for example, in some embodiments, whole genome shotgun sequencing), ribosomal gene sequencing, rRNA sequencing or other sequencing based methods. See, e.g., Thomas et al., 2012, “Metagenomics—A guide from sampling to data analysis,” Microbial Informatics and Experimentation 2(1):3; Qin et al., 2009. “A human gut microbial gene catalogue established by metagenomic sequencing,” Nature 464 (7285): 59-65. For example, in some embodiments, metagenomic sequence reads (i.e. sequence fragments) obtained from a sequencing method are mapped against a comprehensive database of complete, sequenced genomes of all the defined microbial strains comprising a gut community.

As used herein, “molecularly identified” (and grammatical variants thereof, e.g., “molecular identification”) refers to characterization of a microbial species for unique identification. In some embodiments, molecular identification can be 16S rRNA sequencing, whole genome sequencing, Matrix-Assisted Laser Desorption/Ionization Time-Of-Flight Mass Spectrometry (MALDI-TOF MS), liquid chromatography-mass spectrometry (LC-MS) or similar analytical assay capable of differentiating one microbial species from another microbial species. In some embodiments, species identification is done on the level of strain identification. In some embodiments, strain identification is achieved through whole genome shotgun metagenomic sequencing. As used herein, whole genome shotgun metagenomic sequencing refers to a method of sequencing polynucleotides in parallel and with high sequence coverage from a plurality of genomic regions from a complex sample comprising a plurality of microbial species.

### 5. In Vitro and Metabolic Phenotype

As used herein an “in vitro phenotype” refers to a characteristic, such as a metabolic phenotype, of a microbial community that can be measured in vitro. In one embodiment a microbial community is recovered from the gut of an animal. In one embodiment a microbial community is recovered from a fecal sample. In one embodiment a microbial community is an artificial community or a high-complexity defined gut microbial community.

“Metabolic phenotype” is a property of a microbial strain or a microbial community. In one aspect, a metabolic phenotype refers to the ability of a microbial strain or microbial community to transform one or more first compound(s) into one or more second compound(s). In one example a first compound is enzymatically converted by the microbe or community into a second compound, and the metabolic phenotype is an increase in the amount of the second compound. In some embodiments, metabolic phenotypes include metabolization of a-mannan (yeast), acetate, agarose, alanine, alginate, anthocyanin, arabinan, arabinogalactan, arabinoxylan, arginine, asparagine, Aspartate, b-glucans, butyrate, carrageenan, chitin, chlorogenic acids, chondroitin sulfate, cinnamic acid, Cysteine, dextran (40), Dihydrochalcones, Enterodiol, flavan-3-ols, flavanones, flavones, flavonols, folate, formate, galactomannan (carob), galacturonan (homo), galacturonate, glucomannan (konjac), glutamate, Glutamine, Glycine, Histidine, hyaluronan, hydrogen, hydroxycinnamic acids, hydroxyproline, inulin, isoflavones/isoflavanones, Isoleucine, lactate, laminarin, Leucine, levan, Lysine, Methionine, mucin O-linked glycans, Ornithine, Phenylalanine, porphyran, Proline, propionate, rhamnogalacturonan I, rhamnogalacturonan II, Secoisolariciresinol diglucoside, Serine, starch (potato), starch (structure 1), thiamine, Threonine, tryptophan, Tyrosine, Valine, xyloglucan, and xylooligosaccharides (XOS). For example, in some embodiments, one or more of the defined microbial strains of the high-complexity defined gut microbial community metabolizes at least one, at least 2, at least 3, at least 4, at least 5, at least 6, at least 7, at least 8, at least 9, at least 10, at least 11, at least 12, at least 13, at least 14, at least 15, at least 20, at least 25, at least 30, at least 40, at least 50, at least 60, or all of the substrates described above.

In some embodiments, metabolic phenotypes include the production of formate, acetate, propionate, butyrate, isobutyrate, valerate, isovalerate, 2-methylbutyrate, caporate, isocaporate, 3-methylvaleric acid, L-phenylalanine, 3-phenylpropionic acid, phyenypyruvate, DL-3-phenyllactic acid, trans-cinnamic acid, phenyllactic acid, phenethylamine, L-tyrosine, 3-(4-hydroxyphenyl)propionic acid, 3-(4-hydroxyphenyl) pyruvic acid, DL-p-hydroxyphenyl lactic acid, p-coumaric acid, 4-hydroxyphenyl acetic acid, tyramine, phenol, p-cresol, 4-ethylphenol, 4-vinylphenol, 4-hydroxybenzoic acid, L-tryptophan, 3-indolepropionic acid, 3-indolepyruvic acid, DL-indole-3-lactic acid, trans-3-indoleacrylic acid, 3-indoleacetic acid, tryptamine, indole, skatol, indole-3-carboxylic acid, indole-3-carboxyaldehyde, N-acetyl-L-phenylalanine, phenylpropionylglycine, 3-(3-hydroxyphenyl) propionic acid, cinnamoylglycine, phenylacetylglycine, phenylacetylglutamine, hippuric acid, 2-hydroxyhippuric acid, 3-hydroxyhippuric acid, 4-hydroxyhippuric acid, 4-hydroxyphenylacetylglycine, phenyl sulfate, phenyl glucuronide, p-cresol sulfate, p-cresol glucuronide, 4-ethylphenol sulfate, 4-ethylphenol glucuronide, N-acetyl-L-tryptophan, 5-hydroxy-L-typtophan, N-acetyl serotonin, 3-indolepriopionylglycine, indolyl-3-acryloylglycine, indoxyl sulfate, indoxyl glucuronide, 5-hydroxyindole-3-acetic acid, indoleacetylglycine, lithocholic acid, murocholic acid, ursodeoxycholic acid, hyodeoxycholic acid, deoxycholic acid, 7-oxocholic acid, ω-muricholic acid, α-muricholic acid, β-muricholic acid, γ-muricholic acid, 7βcholic acid, taurolithocholic acid, tauroursodeoxycholic acid, taurohyodeoxycholic acid, taurochenodeoxycholic acid, taurodeoxycholic acid, tauro-β-muricholic acid, tauro-ω-muricholic acid, and taurocholic acid. For example, in some embodiments, one or more of the defined microbial strains of the high-complexity defined gut microbial community produces at least one, at least 2, at least 3, at least 4, at least 5, at least 6, at least 7, at least 8, at least 9, at least 10, at least 11, at least 12, at least 13, at least 14, at least 15, at least 20, at least 25, at least 30, at least 40, at least 50, at least 60, at least 70, at least 80, or all of the metabolites described above.

In some embodiments, metabolic phenotypes include the encoding the enzymes catalyzing all reactions of any one or more of the 1CMET2-PWY, 2.6.1.32-RXN, AEROBACTINSYN-PWY, ALACAT2-PWY, ALADEG-PWY, ALANINE-DEG3-PWY, ALANINE-SYN2-PWY, ALANINE-VALINESYN-PWY, ANAPHENOXI-PWY, ARGASEDEG-PWY, ARGDEG-III-PWY, ARGDEG-IV-PWY, ARGDEGRAD-PWY, ARGDEG-V-PWY, ARG-GLU-PWY, ARGININE-SYN4-PWY, ARG-PRO-PWY, ARGSYNBSUB-PWY, ARGSYN-PWY, ASPARAGINE-BIOSYNTHESIS, ASPARAGINE-DEG1-PWY, ASPARAGINE-DEG1-PWY-1, ASPARAGINESYN-PWY, ASPARTATE-DEG1-PWY, ASPARTATESYN-PWY, ASPASN-PWY, ASPSYNII-PWY, AST-PWY, BETA-ALA-DEGRADATION-I-PWY, CAMALEXIN-SYN, CITRULBIO-PWY, CITRULLINE-DEG-PWY, COA-PWY, CODH-PWY, CYSTEINE-DEG-PWY, CYSTSYN-PWY, DAPLYSINESYN-PWY, ENTBACSYN-PWY, ETHYL-PWY, FAO-PWY, FERMENTATION-PWY, GLNSYN-PWY, GLUDEG-I-PWY, GLUGLNSYN-PWY, GLUTAMATE-DEG1-PWY, GLUTAMATE-SYN2-PWY, GLUTAMINDEG-PWY, GLUTAMINEFUM-PWY, GLUTATHIONESYN-PWY, GLUTDEG-PWY, GLUTORN-PWY, GLUTSYNIII-PWY, GLUTSYN-PWY, GLYCGREAT-PWY, GLYSYN-ALA-PWY, GLYSYN-PWY, GLYSYN-THR-PWY, HISDEG-PWY, HISHP-PWY, HISTDEG-PWY, HISTSYN-PWY, HOMOCYSDEGR-PWY, HOMOSER-METSYN-PWY, HOMOSERSYN-PWY, HSERMETANA-PWY, HYDROXYPRODEG-PWY, ILEUDEG-PWY, ILEUSYN-PWY, LARABITOLUTIL-PWY, LCYSDEG-PWY, LEU-DEG2-PWY, LEUSYN-PWY, LYSDEGII-PWY, LYSINE-AMINOAD-PWY, LYSINE-DEG1-PWY, MALATE-ASPARTATE-SHUTTLE-PWY, METH-ACETATE-PWY, METHANOGENESIS-PWY, METHIONINE-DEG1-PWY, MGLDLCTANA-PWY, ORN-AMINOPENTANOATE-CAT-PWY, ORNDEG-PWY, P101-PWY, P162-PWY, P163-PWY, P181-PWY, P261-PWY, P283-PWY, P401-PWY, P541-PWY, PHENYLALANINE-DEG1-PWY, PHESYN, PHOSLIPSYN2-PWY, PHOSPHONOTASE-PWY, PROSYN-PWY, PROUT-PWY, PWY0-1021, PWY0-1221, PWY0-1299, PWY0-1303, PWY0-1305, PWY0-1313, PWY0-1317, PWY0-1321, PWY0-1338, PWY0-1347, PWY0-1355, PWY0-1356, PWY0-1534, PWY0-1544, PWY0-1565, PWY0-1576, PWY0-1577, PWY0-1578, PWY0-1585, PWY0-1601, PWY0-42, PWY0-461, PWY0-823, PWY0-901, PWY-1, PWY-1061, PWY-1121, PWY-1186, PWY1-2, PWY-1263, PWY-1622, PWY-1722, PWY-1781, PWY-181, PWY-1881, PWY-1962, PWY-1981, PWY1F-467, PWY1F-FLAVSYN, PWY1G-0, PWY-2021, PWY-2161, PWY-2181, PWY-2201, PWY-2821, PWY-2941, PWY-2942, PWY-3, PWY-3022, PWY-3081, PWY-3161, PWY-3162, PWY-3181, PWY-3301, PWY-3341, PWY-3385, PWY-3461, PWY-3462, PWY-3581, PWY-361, PWY-3661, PWY-3661-1, PWY-381, PWY-3841, PWY-3941, PWY3DJ-12, PWY3O-4107, PWY3O-4108, PWY-4, PWY-40, PWY-4002, PWY-4041, PWY-4201, PWY-4281, PWY-43, PWY-4321, PWY-4341, PWY-4361, PWY-46, PWY490-3, PWY490-4, PWY-4981, PWY-4983, PWY-4984, PWY4FS-6, PWY-5, PWY-5022, PWY-5024, PWY-5028, PWY-5029, PWY-5030, PWY-5031, PWY-5041, PWY-5048, PWY-5049, PWY-5057, PWY-5075, PWY-5076, PWY-5078, PWY-5079, PWY-5081, PWY-5082, PWY-5087, PWY-5097, PWY-5101, PWY-5103, PWY-5104, PWY-5108, PWY-5109, PWY-5129, PWY-5135, PWY-5136, PWY-5151, PWY-5154, PWY-5155, PWY-5159, PWY-5176, PWY-5188, PWY-5189, PWY-5196, PWY-5199, PWY-5207, PWY-5250, PWY-5254, PWY-5265, PWY-5280, PWY-5283, PWY-5290, PWY-5297, PWY-5298, PWY-5311, PWY-5314, PWY-5316, PWY-5319, PWY-5324, PWY-5329, PWY-5331, PWY-5332, PWY-5364, PWY-5381, PWY-5382, PWY-5386, PWY-5394, PWY-5399, PWY-5436, PWY-5437, PWY-5441, PWY-5443, PWY-5458, PWY-5467, PWY-5468, PWY-5473, PWY-5474, PWY-5494, PWY-5497, PWY-5499, PWY-5629, PWY-5651, PWY-5653, PWY-5665, PWY-5669, PWY-5675, PWY-5679, PWY-5686, PWY-5710, PWY-5736, PWY-5737, PWY-5739, PWY-5740, PWY-5742, PWY-5747, PWY-5748, PWY-5751, PWY-5754, PWY-5766, PWY-5770, PWY-5784, PWY-5788, PWY-5797, PWY-5800, PWY-581, PWY-5811, PWY-5818, PWY-5826, PWY-5877, PWY-5883, PWY-5886, PWY-5912, PWY-5913, PWY-5921, PWY-5936, PWY-5940, PWY-5958, PWY-5963, PWY-5968, PWY-5978, PWY-5980, PWY-5990, PWY-6003, PWY-6004, PWY-601, PWY-6030, PWY-6039, PWY-6045, PWY-6052, PWY-6053, PWY-6054, PWY-6055, PWY-6068, PWY-6069, PWY-6082, PWY-6120, PWY-6121, PWY-6122, PWY-6123, PWY-6124, PWY-6133, PWY-6134, PWY-6141, PWY-6143, PWY-6148, PWY-6151, PWY-6160, PWY-6173, PWY-6196, PWY-6219, PWY-622, PWY-6220, PWY-6233, PWY-6273, PWY-6277, PWY-6281, PWY-6307, PWY-6309, PWY-6313, PWY-6318, PWY-6320, PWY-6321, PWY-6322, PWY-6324, PWY-6328, PWY-6334, PWY-6339, PWY-6343, PWY-6344, PWY-6345, PWY-6346, PWY-6375, PWY-6376, PWY-6381, PWY-6386, PWY-6387, PWY-6397, PWY-6403, PWY-6407, PWY-6408, PWY-6409, PWY-6431, PWY-6435, PWY-6444, PWY-6455, PWY-6456, PWY-6457, PWY-6466, PWY-6471, PWY-6473, PWY-6481, PWY-6486, PWY-6493, PWY-6495, PWY-6511, PWY-6512, PWY-6519, PWY-6533, PWY-6535, PWY-6536, PWY-6537, PWY-6543, PWY-6549, PWY-6559, PWY-6562, PWY-6572, PWY-6573, PWY-6574, PWY-6578, PWY-6588, PWY-6614, PWY-6627, PWY66-301, PWY66-375, PWY-6638, PWY66-391, PWY-6642, PWY66-420, PWY66-421, PWY66-425, PWY66-426, PWY66-428, PWY-6643, PWY-6661, PWY-6673, PWY-6682, PWY-6690, PWY-6696, PWY-6711, PWY-6717, PWY-6720, PWY-6724, PWY-6728, PWY-6731, PWY-6735, PWY-6749, PWY-6769, PWY-6771, PWY-6772, PWY-6773, PWY-6781, PWY-6784, PWY-6790, PWY-6791, PWY-6802, PWY-6807, PWY-6808, PWY-6813, PWY-6815, PWY-6816, PWY-6817, PWY-6818, PWY-6821, PWY-6822, PWY-6823, PWY-6831, PWY-6832, PWY-6834, PWY-6840, PWY-6845, PWY-6853, PWY-6854, PWY-6855, PWY-6891, PWY-6892, PWY-6896, PWY-6898, PWY-6902, PWY-6907, PWY-6908, PWY-6920, PWY-6922, PWY-6936, PWY-6942, PWY-6949, PWY-6953, PWY-6955, PWY-6963, PWY-6964, PWY-6965, PWY-6968, PWY-6969, PWY-6981, PWY-6982, PWY-6986, PWY-6994, PWY-7000, PWY-701, PWY-7014, PWY-7015, PWY-7016, PWY-7018, PWY-7019, PWY-702, PWY-7022, PWY-7025, PWY-7028, PWY-7040, PWY-7046, PWY-7052, PWY-7054, PWY-7064, PWY-7072, PWY-7088, PWY-7090, PWY-7097, PWY-7104, PWY-7115, PWY-7117, PWY-7118, PWY-7126, PWY-7147, PWY-7153, PWY-7158, PWY-7176, PWY-7177, PWY-7185, PWY-7186, PWY-7219, PWY-7221, PWY-7234, PWY-7246, PWY-7248, PWY-7250, PWY-7255, PWY-7274, PWY-7275, PWY-7282, PWY-7288, PWY-7297, PWY-7304, PWY-7315, PWY-7316, PWY-7318, PWY-7342, PWY-7351, PWY-7356, PWY-7376, PWY-7377, PWY-7383, PWY-7387, PWY-7397, PWY-7398, PWY-7400, PWY-7414, PWY-7425, PWY-7430, PWY-7432, PWY-7440, PWY-7441, PWY-7456, PWY-7467, PWY-7498, PWY-7501, PWY-7506, PWY-7510, PWY-7514, PWY-7518, PWY-7520, PWY-7525, PWY-7531, PWY-7532, PWY-7533, PWY-7536, PWY-7542, PWY-7543, PWY-7547, PWY-7549, PWY-7550, PWY-7555, PWY-7561, PWY-7565, PWY-7570, PWY-7571, PWY-7600, PWY-7605, PWY-761, PWY-7612, PWY-7626, PWY-7645, PWY-7648, PWY-7649, PWY-7650, PWY-7665, PWY-7667, PWY-7668, PWY-7669, PWY-7671, PWY-7674, PWY-7688, PWY-7690, PWY-7693, PWY-7694, PWY-7701, PWY-7704, PWY-7706, PWY-7708, PWY-7717, PWY-7718, PWY-7719, PWY-7733, PWY-7734, PWY-7735, PWY-7737, PWY-7751, PWY-7761, PWY-7765, PWY-7767, PWY-7769, PWY-7770, PWY-7782, PWY-7790, PWY-7791, PWY-7793, PWY-7797, PWY-7811, PWY-7814, PWY-7822, PWY-7824, PWY-7826, PWY-7842, PWY-7850, PWY-7851, PWY-7855, PWY-7860, PWY-7861, PWY-7863, PWY-7867, PWY-7870, PWY-7880, PWY-7888, PWY-7889, PWY-7891, PWY-7892, PWY-7897, PWY-7901, PWY-7904, PWY-7907, PWY-7909, PWY-7910, PWY-7913, PWY-7917, PWY-7930, PWY-7931, PWY-7936, PWY-7953, PWY-7955, PWY-7956, PWY-7957, PWY-7958, PWY-7959, PWY-7960, PWY-7962, PWY-7977, PWY-7985, PWY-7986, PWY-7987, PWY-7988, PWY-7990, PWY-8002, PWY-8003, PWY-8006, PWY-8007, PWY-8008, PWY-8009, PWY-801, PWY-8010, PWY-8011, PWY-8013, PWY-8014, PWY-8015, PWY-8016, PWY-8017, PWY-8024, PWY-8032, PWY-8040, PWY-8043, PWY-8045, PWY-8071, PWY-8072, PWY-8080, PWY-8081, PWY-8082, PWY-8083, PWY-8086, PWY-8088, PWY-842, PWY-861, PWY-862, PWY8J2-1, PWY8J2-22, PWYDQC-4, PWYG-321, PWY-I9, PWYQT-4450, PWYQT-4476, PYRIDNUCSAL-PWY, PYRIDNUCSYN-PWY, PYRIDOXSYN-PWY, SAM-PWY, SERDEG-PWY, SERSYN-PWY, SPHINGOLIPID-SYN-PWY, TAURINEDEG-PWY, THRDLCTCAT-PWY, THREONINE-DEG2-PWY, TRNA-CHARGING-PWY, TRPCAT-PWY, TRPIAACAT-PWY, TRPKYNCAT-PWY, TRPSYN-PWY, TRYPDEG-PWY, TYRFUMCAT-PWY, TYRSYN, UDPNACETYLGALSYN-PWY, UDPNAGSYN-PWY, VALDEG-PWY, and VALSYN-PWY MetaCyc pathways. For example, in some embodiments, one or more of the defined microbial strains of the high-complexity defined gut microbial community utilizes at least one, at least 2, at least 3, at least 4, at least 5, at least 6, at least 7, at least 8, at least 9, at least 10, at least 11, at least 12, at least 13, at least 14, at least 15, at least 20, at least 25, at least 30, at least 40, at least 50, at least 60, at least 70, at least 80, at least 90, at least 100, at least 125, at least 150, at least 175, at least 200, at least 250, at least 300, at least 350, at least 400, at least 450, at least 500, at least 550, at least 600, at least 650, or all of the MetaCyc metabolic pathways described above.

### 6. Microbial Community Backfill

“Backfill” methods for producing high-complexity defined gut microbial communities are described in International Application Number PCT/US2019/062,689, which is incorporated herein in its entirety. Backfill methods include “in vitro backfill” and “in vivo backfill.” In vitro backfill and in vivo backfill may be used in combination as described below. In some embodiments, only in vitro backfill is used to produce a community. In some embodiments only in vivo backfill is performed to produce a community. The specification also describes compositions used in, or produced by, these backfill processes.

For convenience, the term “backfilling” is used to describe the process of carrying out an in vitro or in vivo backfill, and the term “backfilled community” refers to a community produced by a backfill process.

### 6.1 Producing a Complex Community by In Vitro Backfilling

In one aspect, the invention involves producing a complex microbial community by in vitro backfilling. A community produced by one or more rounds of in vitro backfilling may be used as the starting stock for one or more rounds of in vivo backfilling.

### 6.2 The Microbial Pantry

As discussed below, a backfill process includes several steps in which an artificial community is prepared by combining several individually selected bacterial strains in the same aliquot. We have designed an initial collection of 109 organisms found in the human gut (including 104 bacterial strains most prevalent in the population and 4 archaea strains). In one aspect, the invention provides, as a useful tool for practicing the backfill method, a “Microbial Pantry,” i.e. an array, such as a multiwell plate, of aliquots containing clonal isolates in which a substantial portion of the strains in Table 7, e.g., at least 80, at least 90, at least 95, or at least 100 strains, are contained in aliquots of the array. In some embodiments the array is a multiwell plate. Also contemplated is a system in which any combination of individual strains in the “pantry” can be automatically robotically retrieved and combined in an aliquot. Thus, in one aspect the invention includes a system comprising an array and a robot under control of a computer for transferring bacteria. The term “Microbial Pantry” can also refer to a collection of clonal aliquots (e.g., tubes) together containing at least a substantial portion of strains listed in Table 7 even if not physically associated in an array, provided the aliquots are in the same location such that any combination of strains can be retrieved. A Microbial Pantry is typically stored frozen until use. In some cases microorganisms are provided as spores.

TABLE 7
Exemplary Strains of a Microbial Pantry

| | |
|---|---|
| Alistipes putredinis DSM 17216 | Clostridium scindens ATCC 35704 |
| Acidaminococcus fermentans DSM 20731 | Clostridium sp. L2-50 |
| Acidaminococcus sp. D21 | Clostridium sp. M62/1 |
| Akkermansia muciniphila ATCC BAA-835 | Clostridium spiroforme DSM 1552 |
| Anaerococcus lactolyticus DSMZ 7456 | Clostridium sporogenes ATCC 15579 |
| Anaerofustis stercorihominis DSM 17244 | Collinsella aerofaciens ATCC 25986 |
| Anaerostipes caccae DSM 14662 | Collinsella stercoris DSM 13279 |
| Anaerotruncus colihominis DSM 17241 | Coprococcus comes ATCC 27758 |
| Bacteroides capillosus ATCC 29799 | Coprococcus eutactus ATCC 27759 |
| Bacteroides cellulosilyticus DSM 14838 | Desulfovibrio piger ATCC 29098 |
| Bacteroides coprocola DSM 17136 | Dialister invisus DSM 15470 |
| Bacteroides coprophilus DSM 18228 | Dorea formicigenerans ATCC 27755 |
| Bacteroides dorei 5_1_36/D4 (HM 29) | Dorea longicatena DSM 13814 |
| Bacteroides dorei DSM 17855 | Eggerthella lenta DSM 2243 |
| Bacteroides eggerthii DSM 20697 | Ethanoligenens harbinense DSMZ 18485 |
| Bacteroides finegoldii DSM 17565 | Eubacterium biforme DSM 3989 |
| Bacteroides fragilis 3_1_12 | Eubacterium dolichum DSM 3991 |
| Bacteroides intestinalis DSM 17393 | Eubacterium eligens ATCC 27750 |
| Bacteroides ovatus ATCC 8483 | Eubacterium hallii DSM 3353 |
| Bacteroides pectinophilus ATCC 43243 | Eubacterium rectale ATCC 33656 |
| Bacteroides plebeius DSM 17135 | Eubacterium siraeum DSM 15702 |
| Bacteroides sp. 1_1_6 | Eubacterium ventriosum ATCC 27560 |
| Bacteroides sp. 2_1_16 | Faecalibacterium prausnitzii A2-165 |
| Bacteroides sp. 2_1_22 | Granulicatella adiacens ATCC 49175 |
| Bacteroides sp. 3_1_19 | Holdemania filiformis DSM 12042 |
| Bacteroides sp. 4_3_ 47FAA | Lactobacillus ruminis ATCC 25644 |
| Bacteroides sp. 9_1_ 42FAA | Lactococcus lactis DSMZ 20729 |
| Bacteroides sp. D2 | Mitsuokella multacida DSM 20544 |
| Bacteroides stercoris ATCC 43183 | Olsenella uli DSM 7084 |
| Bacteroides stercoris DSMZ 19555 | Parabacteroides distasonis ATCC 8503 |
| Bacteroides thetaiotaomicron VP1-5482 | Parabacteroides johnsonii DSM 18315 |
| Bacteroides uniformis ATCC 8492 | Parabacteroides merdae DSMZ 19495 |
| Bacteroides vulgatus ATCC 8482 | Parabacteroides sp. D13 |
| Bacteroides xylanisolvens DSMZ 23964 | Prevotella buccae D17 |
| Bifidobacterium adolescentis L2-32 | Prevotella buccalis DSMZ 20616 |
| Bifidobacterium longum infantis ATCC | Prevotella copri DSM 18205 |
| 55813 | Roseburia intestinalis L1-82 |
| Bifidobacterium pseudocatenulatum DSM | Roseburia inulinivorans DSM 16841 |
| 20438 | Ruminococcus albus strain 8 |
| Bilophila wadsworthia DSM 11045 | Ruminococcus bromii ATCC 27255 |
| Blautia hansenii DSM 20583 | Ruminococcus flavefaciens FD 1 |
| Blautia hydrogenotrophica DSM 10507 | Ruminococcus gnavus ATCC 29149 |
| Bryantella formatexigens DSM 14469 | Ruminococcus lactaris ATCC 29176 |
| Butyrivibrio crossotus DSM 2876 | Ruminococcus obeum ATCC 29174 |
| Catenibacterium mitsuokai DSM 15897 | Ruminococcus torques ATCC 27756 |
| Clostridium asparagiforme DSM 15981 | Slackia exigua DSMZ 15923 |
| Clostridium bartlettii DSM 16795 | Slackia heliotrinireducens DSM 20476 |
| Clostridium bolteae ATCC BAA-613 | Solobacterium moorei DSM 22971 |
| Clostridium hathewayi DSM 13479 | Streptococcus thermophilus LMD-9 |
| Clostridium hylemonae DSM 15053 | Subdoligranulum variabile DSM 15176 |
| Clostridium leptum DSM 753 | Veillonella dispar ATCC 17748 |
| Clostridium methylpentosum DSM 5476 | Veillonella sp. 6_1_27 |
| Clostridium nexile DSM 1787 | Methanobrevibacter smithii Balch and Wolfe |
| Clostridium saccharolyticum WM1 DSMZ | 1981 strain B181 (DSMZ 11975) |
| 2544 | Methanobrevibacter smithii Balch and Wolfe |
| Methanobrevibacter smithii Balch and Wolfe | 1981 strain PS (DSMZ 861) |
| 1981 strain ALI (DSMZ 2375) | |
| Methanobrevibacter smithii Balch and Wolfe | |
| 1981 strain Fl (DSMZ 2374) | |

In addition to the strains listed in Table 7, it is contemplated that other bacterial strains (which will typically be anaerobes or facultative anaerobes) may be used in backfill methods, including non-naturally occurring genetically modified organisms. Exemplary genetic modifications include, without limitation, mutation or knock out of enzyme-encoding genes and expression of heterologous genes.

### 6.3 First Backfill Community

Backfilling is an iterative process. A “first backfill community” is prepared by combining strains of a “scaffold community” with “backfill strains.” Broadly speaking, and without intending to be bound by a particular mechanism, the scaffold community is a combination of strains selected to produce a desired metabolic phenotype. Backfill strains are a combination of strains selected to include strains that contribute to the stability of the first backfill community in vitro and contribute to the stability of a resulting transplantable community in the human gut. Without intending to be bound by a particular mechanism, it is believed that the backfill processes increase the complexity of the community and that communities with higher complexity tend to inhabit more niches in the gut and be more stable.

### 6.4 Scaffold Community

A scaffold community comprises a plurality of strains common in the human gut microbiome. A given scaffold community typically contains 5-100 strains, usually 10-30 strains. The scaffold community may comprise one or more strains listed in Table 7 such as, for example, at least 5, at least 10, at least 20, or at least 30 strains listed in Table 7. In some approaches, at least 50%, 75%, 90% or all of the strains in a scaffold community are selected from Table 7.

The scaffold community is selected to exhibit a desired phenotype, typically a desired metabolic phenotype. A “metabolic phenotype” of a community, as described above, refers to the production or consumption of metabolites by the community. An exemplary metabolic phenotype is the ability to increase or decrease the concentration of a compound or compounds in the environment as a result of microbial metabolic processes. For example, a scaffold community comprising Clostridium sporogenes may consume phenylalanine and produce tyrosine, in which case the metabolic phenotype could be “produce tyrosine.” Similarly, a community comprising Proteus mirabilis in an environment containing urea may decrease the concentration of urea and increase the concentration of ammonia, and a community comprising Bacillus subtilis in an environment containing sucrose may decrease the concentration of sucrose and increase the concentration of glucose. Importantly, however, these simple illustrations vastly oversimplify the metabolic processes that occur in a microbial ecosystem. For example, the metabolic product of a first member of a microbial community may be a metabolic substrate for a second member of the community, or the metabolic product of one member of the microbial community may be a transcriptional activator in another microbe or, alternatively, may be toxic to the other microbe. In a complex microbial ecosystem comprising hundreds of different strains, it is not possible, using current methods, to accurately predict the network of interactions of strains, metabolites, and environmental factors of a particular microbial ecosystem even if the identity of each species present is known. Further, unless or until a microbial ecosystem is at homeostasis, the combination of strains in the population will be unstable and may change in unpredictable ways, which may change the metabolic phenotype of the community.

6.5 Creating First in vitro Backfill Communities by Adding Backfill Strains to Scaffold Communities

To create a first in vitro backfill community, the designed scaffold community is supplemented with additional microbial strains referred to as “backfill strains.” For example, each scaffold community may be combined with 35 to 495 additional strains. In some embodiments, each scaffold community may be combined with between 40 and 400, between 40 and 300, between 40 and 200, between 40 and 150, between 40 and 140, between 40 and 130, between 40 and 120, between 40 and 110, between 40 and 100, between 50 and 400, between 50 and 300, between 50 and 200, between 50 and 150, between 50 and 140, between 50 and 130, between 50 and 120, between 50 and 110, between 50 and 100, between 60 and 400, between 60 and 300, between 60 and 200, between 60 and 150, between 60 and 140, between 60 and 130, between 60 and 120, between 60 and 110, between 60 and 100, between 70 and 500, between 70 and 400, between 70 and 300, between 70 and 200, between 70 and 150, between 70 and 140, between 70 and 130, between 70 and 120, between 70 and 110, between 70 and 100, between 80 and 400, between 80 and 300, between 80 and 200, between 80 and 150, between 80 and 140, between 80 and 130, between 80 and 120, between 80 and 110, between 80 and 100, between 90 and 400, between 90 and 300, between 90 and 200, between 90 and 150, between 90 and 140, between 90 and 130, between 90 and 120, between 90 and 110, between 90 and 100, between 100 and 400, between 100 and 300, between 100 and 200, between 100 and 150, between 100 and 140, between 100 and 130, between 100 and 120, or between 100 and 110 defined microbial strains. The backfill strains and the strains of the scaffold community may be combined in any order. For example, backfill strains can be added in a single batch to all of the scaffold community strains. Alternatively, subsets of scaffold community strains may be combined with subsets of the backfill strains, in any desired sequence.

### 6.6 Parallel Backfill Communities

In vitro backfill methods are carried out according to the methods disclosed herein, by testing many different lineages and combinations in parallel as described in greater detail below. Although in principle a single first in vitro backfill community can be produced by combining a single scaffold community with backfill strains, the robustness of the method arises, in part, from parallel processing of multiple communities. Typically a plurality of first in vitro backfill communities designed to exhibit a predetermined metabolic phenotype are produced (e.g., typically from 2 to 100 communities, and generally at least 5, at least 10 or at least 15 communities) by combining scaffold communities and backfill communities. In one approach, multiple aliquots of one scaffold community are used. In one approach multiple different scaffold communities are used, where the communities are designed for the same metabolic phenotype but with different (sometimes only slightly different) combinations of strains. In each approach, one combination of backfill strains, or multiple different combinations of backfill strains may be used. Thus, in the in vitro backfill process, multiple first backfilled communities may be created, propagated, and assayed in parallel.

The number of different first backfill communities assayed in parallel can range from 2 to 100 or more. Typically the number is greater than 5, greater than 10, greater than 25, greater then 50, or greater than 100.

### 6.7 Culturing In Vitro Backfill Communities

The first backfill communities, as well as subsequent in vitro backfill communities (described below) are cultured for a period of time and then are assessed as described below. The strains may be cultured for 2 hours to 10 days, although longer or shorter times can be used. For example, the backfill communities can be cultured for 1 to 72 hours, e.g., 12 to 72 hours, 12 to 48 hours, or 24 to 48 hours. Typically the strains are cultured in an environment that mimics the temperature of the human gut (e.g., 36-38° C.) and low pO₂ (e.g., under anaerobic conditions). Preferably a single universal culture medium is used, which may be designed to approach the conditions encountered in the mammalian (e.g., human) gut.

### 6.8 Assessing and Ranking In Vitro Backfill Communities

At the end of a culture period, or at multiple times during a culture period, one or more properties of the first backfill communities, as well as subsequent in vitro backfill communities, can be assessed. For illustration and not limitation, exemplary properties that can be assessed include a metabolic phenotype and antibiotic resistance.

### 6.9 Assessing Strain Composition

At the end of a culture period, or at any desired time during culture, the strain composition of a backfill community can be determined. Strain composition can be determined by metagenomic analysis, by quantitative assessments such as qPCR, using microbiological techniques such as colony counting, or combinations of methods. In one aspect, the abundance, or relative proportions, of individual strains can be measured.

### 6.10 Assessing Changes in Strain Composition

By determining the strain composition of a community at different timepoints, changes in composition can be detected. Some strains “drop out” during culture and/or during in vivo backfill. Changes in strain composition over different rounds or iterations of in vitro or in vivo backfilling, discussed below, can be used as a measure of “Community Composition Stability,” i.e. stability, as defined above.

### 6.11 Assessing Metabolic Phenotype

The metabolic phenotype of a backfill community can be determined at the end of, or during, a culture period. Metabolic phenotype can be assayed in any suitable fashion based on the desired phenotype. For example, in one approach, one or more than one first compound is combined with a community and conversion of the first compound(s) to second compound(s) is measured over time or at an end point. Detection and measurement of compounds or other properties can be made in any of a variety of ways. For example, liquid chromatography mass spectrometry (LC-MS), immunoassay (ELISA), tracing radiolabeled metabolites, etc., may be used to detect compounds produced or consumed by a community. Assays may be carried out under conditions that mimic those of the mammalian (e.g., human) gut, or over multiple conditions that mimic variation in the guts of individuals in a population.

Changes in metabolic phenotype over different rounds or iterations of in vitro or in vivo backfilling, discussed below, can be used as a measure of “Community Phenotype Stability.”

### 6.12 Other Assessments

The backfilled communities may also be tested for antibiotic susceptibility or resistance, contamination, and the like. In some cases, a backfilled community may be challenged with a pathogen or other microorganism to determine whether addition of the, e.g., pathogen perturbs or overgrows the community. In some cases, a backfill community may be introduced into the gut of a humanized mouse to determine whether the community can displace the enteric microbiome.

### 6.13 Ranking Communities

The first, and subsequent, backfill communities may be ranked according to assessed properties such as metabolic phenotype. For example, if the desired community phenotype is production of metabolite X under defined conditions, the ability of the community to produce X, the rate at which X is produced or other kinetic measurements, and the like, can be measured and the Backfill Communities in which the desired phenotype is more robust can be ranked higher than communities in which the desired phenotype is absent or less robust. Multiple properties or criteria can be considered and may be assigned equal or unequal weights and used for ranking.

### 6.14 Selection of Backfilled Communities

As noted above, backfill communities may be ranked according to any combination of properties, weighed in any manner. In one approach, the highest ranked backfill community or communities are selected for further processing. In one approach, the highest ranked community is selected for further processing. In one approach, the highest ranked 1%, 5%, 10% or 25% of communities are processed for further development. In one approach, communities exhibiting properties above a predetermined threshold may be selected for further processing. Communities that are not selected may be discarded.

A backfill community selected for further processing can be called a “selected backfill community.”

### 6.15 Further Processing: Subsequent Backfill Communities

The selected (most highly ranked) first backfill community or communities may be further processed in subsequent iterations, or rounds, of the in vitro backfill process. In one approach, the selected first backfill communities are processed in a manner analogous to the treatment of the scaffold community. In some embodiments, each selected first backfill community is divided into multiple aliquots for parallel processing, and a small number of backfill strains (e.g., 1-50 strains) are added to each aliquot, thereby producing a “subsequent backfill community.” The backfill strains added to each aliquot are not the same for all aliquots of a first backfill community; rather different combinations and different complexities of backfill strains may be added. The process of adding backfill strains to one backfill community (e.g., a first backfill community) to produce a subsequent backfill community can be referred to as “challenging” or “evolving” the community.

The subsequent backfill communities are cultured for a period of time (“culture period”), and at the end of a culture period, or at multiple times during a culture period, one or more properties of the subsequent community is assessed as described above, and subsequent communities are ranked for additional iterations or rounds of further processing. The properties assessed, and used for ranking, in one round of processing may be the same or different from properties assessed in previous or subsequent rounds.

### 6.16 Iterations

When developing a complex community for transplantation, multiple iterations of the backfilling process may be carried out. As used in this context, producing the first backfill community is a first iteration, and subsequent iterations are used to produce subsequent backfill communities are denoted by ordinal numbers (second backfill community, third backfill community, etc.). As used in this context, second or subsequent “iterations” include the process of (1) adding at least one backfill strain to an existing backfill population to produce a next generation population, (2) culturing the next generation population, (3) optionally determining a characteristic of the population.

The number of iterations of producing subsequent backfill communities (i.e. not including the first backfill community) may range from 1 to 20. Typically the number of iterations is in the range 5-10 iterations. In general, there are at least 1, 2, 3, 4, 5, 6, or 7 iterations producing subsequent in vitro backfill communities.

As noted above, a selected backfill community can be divided into multiple aliquots each of which is combined with one or more backfill strains (e.g., where not all aliquots receive the same backfill strains). It is sometimes useful to describe the lineage of a community. In any subsequent backfill iteration, communities produced from the same selected backfill community are referred to as “sibling communities” of each other and as “progeny” of the selected backfill community. The selected backfill community can be referred to as an “ancestor” of the progeny communities.

### 6.17 Producing a Transplantable Community by In Vivo Backfilling

After a final iteration of in vitro backfilling, one or more of the subsequent backfill communities may be identified as having desirable properties (e.g., a desired metabolic phenotype), and may be used as a first in vivo backfill community. The in vivo backfill process parallels the in vitro process described above in several respects. Many of the in vivo backfill steps are the same as, or analogous to, corresponding in vitro steps discussed above. The chief differences are:
the first in vivo backfill community is usually a community produced by in vitro backfill, rather than a scaffold community;
backfill communities are engrafted into a non-human animal (typically a gnotobiotic mouse) rather than cultured in vitro; and
backfill communities are challenged, or evolved, by combining an engrafted backfill community with human fecal transplant material comprising a complex mixture of strains. Optionally, backfill strains may also be administered.

Analogous with the in vitro method, multiple first in vivo backfill communities may be developed in parallel as described in greater detail below. Thus, for example and not limitation, one approach to in vivo backfill includes the following steps:

i. engraft a selected in vitro backfill community into the gut(s) of one mouse or a plurality of mice or other non-human animal;

iia. introduce human fecal transplant material into the gut(s) of the one mouse or the plurality of mice (i.e. challenge the engrafted community) prior to or after step (i);

iib. optionally, backfill strains (e.g., from the Microbial Pantry) may also be administered into the mouse or the plurality of mice;

iii. maintain the mouse or the plurality of mice for a period of time during which time the engrafted and introduced strains colonize the gut, resulting in a “gut community;”

iv. assess one or more properties of the gut communities including composition (i.e. the presence of strains that “jump in” or “drop out” relative to the in vitro backfill community engrafted in step (i);

v. optionally, rank gut communities, and select one or more gut communities for further processing;

vi. for each selected gut community, engraft a plurality of mice with the community; and

vii. challenge the mice in (vi) by introducing human fecal transplant material (as in step ii, above) and carry out additional iterations of steps (ii)-(vi) until a desired endpoint.

Certain aspects of the in vivo backfill method are described in more detail below.

In vivo backfill is usually carried out in gnotobiotic mice, humanized mice, or other mammals (e.g., simians, equines, bovines, porcines, canines, felines, and the like). Gnotobiotic mice are known in the art and commercially available. In some embodiments, in vivo backfill may be carried out in human subjects.

A selected in vitro community or subsequent in vivo communities can be engrafted into mice using standard methods such as gavage.

An engrafted community can be challenged with human fecal material when developing treatments for human patients. Fecal preparations from other species may be used in model systems or in development of treatments for veterinary purposes (see Hu, J et al., 2018, “Standardized Preparation for Fecal Microbiota Transplantation in Pigs,” Front. Microbiol. 9:1328.

The feces donor may be selected or screened for certain characteristics such as the health of the donor.

Fecal material is processed for transplantation using art-known methods. In some cases, fecal material from more than one individual will be pooled for engraftment.

Fecal material may be introduced into the mouse gut by gavage. The engrafted mouse is housed under germ free conditions for 1 day to 4 weeks. This interval may be referred to as the “colonization period.”

At the end of a colonization period, or at multiple times during a colonization period, one or more properties of the first backfill communities, as well as subsequent in vitro backfill communities, can be assessed.

For purposes of assessment, a community may be recovered from the animal (e.g., mouse) gut in any fashion that maintains the integrity of the microbiome including (1) recovery of strains from feces; (2) recovery of gut contents; and (3) recovery of the gut surgically (e.g., by sacrifice of mouse).

The characteristics of the community that may be assayed and suitable methods include those described for in vitro backfill, including changes in strain composition; metabolic phenotype; and/or strain and phenotype stability.

In addition to analysis of the backfill community, the mouse phenotype can be analyzed. Characteristics include the general health and vigor of the mouse, as well as changes in blood or other tissues, such as a change in plasma levels of a metabolite, especially a metabolite related to the desired metabolic phenotype.

The in vivo backfill communities may be ranked according to assessed properties (such as metabolic phenotype). Multiple properties or criteria can be considered and may be assigned equal or unequal weights and used for ranking.

The selected (most highly ranked) in vivo backfill community or communities may be further processed in subsequent iterations, or rounds, of the in vivo backfill process. From 2-10 iterations (usually 2-5, often 2-4, iterations). After a final iteration of in vivo backfilling, one or more in vivo subsequent backfill community may be identified as suitable for use as a therapeutic agent, referred to as a “therapeutic backfill community.”

### 6.18 In Vivo Backfill

In in vivo backfill, one approach is to administer to a non-human animal a defined enteric community that is produced through a series of steps that include the following.
1. Obtaining a first defined microbial community with an in vitro phenotype. Usually the first defined microbial community is a product of in vitro backfill. The in vitro phenotype may be a metabolic phenotype.
2. Engrafting the defined microbial community into the gut of an animal, typically a mouse such as a germ-free mouse. This engrafting step may be carried out in a plurality (i.e. two or more) of animals in parallel.
3. Challenging the animal with a human fecal community (e.g., feces from a human). In this context, “challenging” means introducing the human fecal community into the gut of the animal previously engrafted with the defined microbial community so that the two communities mix. Alternatively, the two communities can be combined prior to engraftment and the mixture engrafted into the animal. The challenged engrafted animal is maintained for a time sufficient to establish in the gut a community comprising microorganisms from both the human fecal community and the defined microbial community, which may be referred to as a “gut community.” The gut community may contain fewer or more strains than the defined microbial community. The gut community may comprise strains contributed from the human fecal community (strains that have “jumped in”). The gut community may not comprise strains (strains that have “dropped out”) that were present in the defined microbial community. If more than one animal is challenged, they may be challenged with the same human fecal preparation or with different human fecal preparations. In one approach, not all of the animals are challenged with the same human fecal community.
4. Carrying out a metagenomic analysis to detect strains in the gut community and determining whether there are or are not differences between the gut community and the defined community. If there are differences (strains have jumped in or dropped out), a new defined microbial community (a “subsequent defined microbial community”) is prepared (e.g., using strains from the microbial pantry and/or other sources). The subsequent defined microbial community is engrafted into an animal (e.g., an animal not previously engrafted) and processed as the first defined microbial community as discussed above. These steps can be repeated for a plurality of iterations. For example, they can be repeated 1, 2, 3, 4, 5 or 6 times (e.g., typically 1-4 times).
5. Carrying out one or more assays to confirm that the gut community retains the desired phenotype (i.e. the phenotype that will provide therapeutic benefit to a patient). Gut communities that do not retain the phenotype are abandoned. In some approaches, multiple different gut communities can be ranked based on the results of the assays, e.g., within communities strongly expressing the phenotype being ranked higher. In some approaches, higher ranked communities are processed further and lower ranked communities are abandoned.
6. If a defined microbial community is stable, e.g., when engrafted and challenged a minimal difference of strains jump in or drop out, and retains the desired phenotype, it may be used as a therapeutic agent. In some approaches, a defined microbial community is deemed stable if fewer than a threshold number of strains jump in and/or fewer than a threshold number of strains drop out. In some embodiments the threshold numbers for jump in and drop out are independently selected from 1, 2, 3, 4, 5, 6, 7, 8, 9 or 10 strains. In some embodiments, the threshold numbers for jump in and drop out are independently selected from 1%, 2%, 3%, 4%, 5%, 6%, 7%, 8%, 9% or 10% of the strains in the engrafted defined microbial community.

### 6.19 Variations

In certain embodiments, a mammal can be engrafted with first in vitro communities (produced by combining a scaffold community with backfill strains) without undertaking an in vitro backfill process.

### 7. Producing a High-Complexity Defined Gut Microbial Community

In some embodiments, a high-complexity defined gut microbial community can be produced by an in vivo backfill process comprising: i) combining a plurality of defined microbial strains; ii) engrafting the combined plurality of defined microbial strains into the gut of an animal to produce an engrafted animal; iii) challenging the engrafted animal with a human fecal sample; iv) maintaining the challenged engrafted animal for a time sufficient for enteric colonization of the animal by microbial strains of the human fecal sample, thereby producing an enteric community in the gut of the animal; v) identifying microbial strains of the enteric community by metagenomic analysis; vi) identifying whether there are differences between the microbial strains comprising the enteric community and the microbial strains comprising the combined plurality of defined microbial strains; vii) if there is a significant difference between the microbial strains comprising the enteric community and the microbial strains comprising the combined plurality of defined microbial strains, adding one or more than one additional defined microbial strain that was not present in step i) to the combined plurality of defined microbial strains, or removing a defined microbial strain that was present in the combined plurality of defined microbial strains of step i), to produce a modified, combined plurality of defined microbial strains and repeating steps ii) to vi) in an animal that has never been engrafted, using the modified, combined plurality of defined microbial strains as the combined plurality of defined microbial strains, and if there are minimal differences, the modified, defined, microbial community in the final step vii) is a high-complexity defined gut microbial community. In some embodiments, defined microbial strains are selected for combining to form a plurality for engraftment based on the metabolic phenotype of the microbial strains. By selecting defined microbial strains having known metabolic phenotypes, high-complexity defined metabolic communities can be formed that have improved engraftment and/or stability in one or more gut niches.

In some embodiments, a high-complexity defined gut microbial community can comprise microbial strains belonging to the phyla consisting of Bacteroidetes, Firmicutes, Actinobacteria. In some embodiments, a high-complexity defined gut microbial community can comprise microbial strains belonging to the phyla consisting of Bacteroidetes, Firmicutes, Actinobacteria, and Proteobacteria. n some embodiments, a high-complexity defined gut microbial community can comprise microbial strains belonging to Bateriodales, Clostridiales, Lactobacillales, Negativicutes, Eggerthellales, Bifidobacteriales, or Proteobacteria.

In certain embodiments, a high-complexity defined gut microbial community can comprise microbial strains selected from, or consist of the microbial strains: Acidaminococcus fermentans DSM 20731, Acidaminococcus sp. D21, Akkermansia muciniphila ATCC BAA-835, Alistipes putredinis DSM 17216, Anaerofustis stercorihominis DSM 17244, Anaerostipes caccae DSM 14662, Anaerotruncus colihominis DSM 17241, Bacteroides caccae ATCC 43185, Bacteroides cellulosilyticus DSM 14838, Bacteroides coprocola DSM 17136, Bacteroides coprophilus DSM 18228, Bacteroides dorei 5_1_36/D4 (HM 29), Bacteroides dorei DSM 17855, Bacteroides eggerthii DSM 20697, Bacteroides finegoldii DSM 17565, Bacteroides fragilis 3_1_12, Bacteroides intestinalis DSM 17393, Bacteroides ovatus ATCC 8483, Bacteroides pectinophilus ATCC 43243, Bacteroides plebeius DSM 17135, Bacteroides sp. 1_1_6, Bacteroides sp. 2_1_16, Bacteroides sp. 2_1_22, Bacteroides sp. 3_1_19, Bacteroides sp. 9_1_42FAA, Bacteroides sp. D2, Bacteroides stercoris ATCC 43183 DSMZ 19555, Bacteroides thetaiotaomicron VPI-5482, Bacteroides uniformis ATCC 8492, Bacteroides vulgatus ATCC 8482, Bacteroides xylanisolvens SD CC 1b->subbed w/ DSMZ 18836, Bifidobacterium adolescentis L2-32, Bifidobacterium breve DSM 20213, Bifidobacterium catenulatum DSM 16992, Bifidobacterium longum infantis ATCC 55813, Bifidobacterium pseudocatenulatum DSM 20438, Blautia hansenii DSM 20583, Blautia hydrogenotrophica DSM 10507, Bryantella formatexigens DSM 14469, Butyrivibrio crossotus DSM 2876, Catenibacterium mitsuokai DSM 15897, Clostridium asparagiforme DSM 15981, Clostridium bartlettii DSM 16795, Clostridium bolteae ATCC BAA-613, Clostridium hathewayi DSM 13479, Clostridium hylemonae DSM 15053, Clostridium leptum DSM 753, Clostridium methylpentosum DSM 5476, Clostridium nexile DSM 1787, Clostridium saccharolyticum WM1 DSMZ 2544, Clostridium scindens ATCC 35704, Clostridium sp. L2-50, Clostridium sp. M62/1, Clostridium spiroforme DSM 1552, Clostridium sporogenes ATCC 15579, Collinsella aerofaciens ATCC 25986, Collinsella stercoris DSM 13279, Coprococcus comes ATCC 27758, Coprococcus eutactus ATCC 27759, Desulfovibrio piger ATCC 29098, Dialister invisus DSM 15470, Dorea formicigenerans ATCC 27755, Dorea longicatena DSM 13814, Eggerthella lenta DSM 2243, Ethanoligenens harbinense YUAN-3 DSMZ 18485, Eubacterium biforme DSM 3989, Eubacterium dolichum DSM 3991, Eubacterium eligens ATCC 27750 DSMZ 3376, Eubacterium hallii DSM 3353, Eubacterium rectale ATCC 33656, Eubacterium siraeum DSM 15702, Eubacterium ventriosum ATCC 27560 DSM 3988, Faecalibacterium prausnitzii A2-165, Granulicatella adiacens ATCC 49175 DSMZ 9848, Holdemania filiformis DSM 12042, Lactobacillus ruminis ATCC 25644, Lactococcus lactis subsp. lactis Il1403->sub DSMZ 20729, Megasphaera DSMZ 102144, Mitsuokella multacida DSM 20544, Olsenella uli DSM 7084, Parabacteroides distasonis ATCC 8503, Parabacteroides johnsonii DSM 18315, Parabacteroides merdae ATCC 43184 DSMZ 19495, Parabacteroides sp. D13, Prevotella buccae D17, Prevotella buccalis ATCC 35310 DSMZ 20616, Prevotella copri DSM 18205, Roseburia intestinalis L1-82, Roseburia inulinivorans DSM 16841, Ruminococcus albus strain 8, Ruminococcus bromii L2-32, Ruminococcus flavefaciens FD 1, Ruminococcus gnavus ATCC 29149, Ruminococcus lactaris ATCC 29176, Ruminococcus obeum ATCC 29174, Ruminococcus torques ATCC 27756, Slackia exigua ATCC 700122 DSMZ 15923, Slackia heliotrinireducens DSM 20476, Solobacterium moorei DSM 22971, Streptococcus thermophilus LMD-9 (ATCC 19258), Subdoligranulum variabile DSM 15176, Veillonella dispar ATCC 17748, Veillonella sp. 3_1_44 HM 64, and Veillonella sp. 6_1_27 HM 49.

In certain embodiments, a high-complexity defined gut microbial community can comprise microbial strains selected from, or consist of the microbial strains: Acidaminococcus fermentans DSM 20731, Acidaminococcus sp. D21, Adlercreutzia equolifaciens DSM 19450, Akkermansia muciniphila ATCC BAA-835, Alistipes finegoldii DSM 17242, Alistipes ihumii AP11, Alistipes indistinctus YIT 12060/DSM 22520, Alistipes onderdonkii DSM 19147, Alistipes putredinis DSM 17216, Alistipes senegalensis JC50/DSM 25460, Alistipes shahii WAL 8301/DSM 19121, Anaerofustis stercorihominis DSM 17244, Anaerostipes caccae DSM 14662, Anaerotruncus colihominis DSM 17241, Bacteroides caccae ATCC 43185, Bacteroides cellulosilyticus DSM 14838, Bacteroides coprocola DSM 17136, Bacteroides coprophilus DSM 18228, Bacteroides dorei 5_1_36/D4 (HM 29), Bacteroides dorei DSM 17855, Bacteroides eggerthii DSM 20697, Bacteroides finegoldii DSM 17565, Bacteroides fragilis 3_1_12, Bacteroides intestinalis DSM 17393, Bacteroides ovatus ATCC 8483, Bacteroides pectinophilus ATCC 43243, Bacteroides plebeius DSM 17135, Bacteroides rodentium DSM 26882, Bacteroides sp. 1_1_6, Bacteroides sp. 2_1_16, Bacteroides sp. 2_1_22, Bacteroides sp. 3_1_19, Bacteroides sp. 9_1_42FAA, Bacteroides sp. D2, Bacteroides stercoris ATCC 43183 DSMZ 19555, Bacteroides thetaiotaomicron VPI-5482, Bacteroides uniformis, ATCC 8492, Bacteroides vulgatus ATCC 8482, Bacteroides xylanisolvens SD CC 1b->subbed w/ DSMZ 18836, Bifidobacterium breve, Bifidobacterium catenulatum DSM 16992, Bifidobacterium pseudocatenulatum DSM 20438, Bilophila wadsworthia ATCC 49260, Blautia hansenii DSM 20583, Blautia hydrogenotrophica DSM 10507, Blautia sp. KLE 1732 (HM 1032), Blautia wexlerae DSM 19850, Bryantella formatexigens DSM 14469, Burkholderiales bacterium 1_1_47, Butyricimonas virosa DSM 23226, Butyrivibrio crossotus DSM 2876, Catenibacterium mitsuokai DSM 15897, Clostridiales bacterium VE202-03, Clostridiales bacterium VE202-14, Clostridiales bacterium VE202-27, Clostridium asparagiforme DSM 15981, Clostridium bartlettii DSM 16795, Clostridium bolteae ATCC BAA-613, Clostridium hathewayi DSM 13479, Clostridium hylemonae DSM 15053, Clostridium leptum DSM 753, Clostridium methylpentosum DSM 5476, Clostridium nexile DSM 1787, Clostridium saccharolyticum WM1 DSMZ 2544, Clostridium scindens ATCC 35704, Clostridium sp. ATCC 29733 VPI C48-50, Clostridium sp. L2-50, Clostridium sp. M62/1, Clostridium spiroforme DSM 1552, Collinsella aerofaciens ATCC 25986, Collinsella stercoris DSM 13279, Coprococcus comes ATCC 27758, Coprococcus eutactus ATCC 27759, Desulfovibrio piger ATCC 29098, Dorea formicigenerans ATCC 27755, Dorea longicatena DSM 13814, Eggerthella lenta DSM 2243, Ethanoligenens harbinense YUAN-3 DSMZ 18485, Eubacterium biforme DSM 3989, Eubacterium dolichum DSM 3991, Eubacterium eligens ATCC 27750 DSMZ 3376, Eubacterium hallii DSM 3353, Eubacterium rectale ATCC 33656, Eubacterium siraeum DSM 15702, Eubacterium ventriosum ATCC 27560 DSM 3988, Faecalibacterium prausnitzii A2-165, Granulicatella adiacens ATCC 49175 DSMZ 9848, Holdemania filiformis DSM 12042, Intestinimonas butyriciproducens DSM 26588, Lactobacillus ruminis ATCC 25644, Megasphaera DSMZ 102144, Mitsuokella multacida DSM 20544, Odoribacter splanchnicus DSM 20712, Olsenella uli DSM 7084, Oscillibacter sp. KLE 1728, Parabacteroides distasonis ATCC 8503, Parabacteroides johnsonii DSM 18315, Parabacteroides merdae ATCC 43184 DSMZ 19495, Parabacteroides sp. D13, Prevotella buccae D17, Prevotella buccalis ATCC 35310 DSMZ 20616, Prevotella copri DSM 18205, Roseburia intestinalis L1-82, Roseburia inulinivorans DSM 16841, Ruminococcus albus strain 8, Ruminococcus bromii ATCC, Ruminococcus flavefaciens FD 1, Ruminococcus gauvreauii DSM 19829, Ruminococcus gnavus ATCC 29149, Ruminococcus lactaris ATCC 29176, Ruminococcus obeum ATCC 29174, Ruminococcus torques ATCC 27756, Slackia exigua ATCC 700122 DSMZ 15923, Slackia heliotrinireducens DSM 20476, Solobacterium moorei DSM 22971, Streptococcus thermophilus LMD-9 (ATCC 19258), Subdoligranulum sp. 4_3_54A2FAA, Subdoligranulum variabile DSM 15176, and Veillonella dispar ATCC 17748.

In certain embodiments, a high-complexity defined gut microbial community can comprise microbial strains selected from, or consist of the microbial strains described in Table 8.

TABLE 8
Exemplary High-Complexity Defined Gut Microbial Community Strains

| | Strain | Source |
|---|---|---|
| Strain | Repository ID | Repository |
| Acidaminococcus fermentans-VR4 | DSM 20731 | DSMZ |
| Acidaminococcus sp.-D21 | HM-81 | BEI |
| Adlercreutzia equolifaciens-FJC-B9 | DSM 19450 | DSMZ |
| Akkermansia muciniphila-Muc [CIP 107961] | ATCC | ATCC |
| | BAA-835 | |
| Alistipes finegoldii-AHN 2437 | DSM 17242 | DSMZ |
| Alistipes indistinctus-JCM 16068, YIT 12060 | DSM 22520 | DSMZ |
| Alistipes onderdonkii-WAL 8169 | DSM 19147 | DSMZ |
| Anaerobutyricum hallii-VPIB4-27 | DSM 3353 | DSMZ |
| Anaerofustis stercorihominis-ATCC BAA-858, CCUG | DSM 17244 | DSMZ |
| 47767, CIP 108481, WAL 14563 | | |
| Anaerostipes caccae-L1-92 | DSM 14662 | DSMZ |
| Anaerotruncus colihominis-277 | DSM 17241 | DSMZ |
| Bacteroides caccae-VPI 3452A [CIP 104201T, JCM 9498] | ATCC 43185 | ATCC |
| Bacteroides cellulosilyticus-CRE21, CCUG 44979 | DSM 14838 | DSMZ |
| Bacteroides coprocola-M16 | DSM 17136 | DSMZ |
| Bacteroides coprophilus-CB42, JCM 13818 | DSM 18228 | DSMZ |
| Bacteroides dorei-175 | DSM 17855 | DSMZ |
| Bacteroides dorei-5_1_36/D4 | HM-29 | BEI |
| Bacteroides eggerthii-ATCC 27754, NCTC 11155 | DSM 20697 | DSMZ |
| Bacteroides finegoldii-199 | DSM 17565 | DSMZ |
| Bacteroides fragilis-3_1_12 | HM-20 | BEI |
| Bacteroides intestinalis-341 | DSM 17393 | DSMZ |
| Bacteroides ovatus-NCTC 11153 | ATCC 8483 | ATCC |
| Bacteroides rodentium-ST28, CCUG 59334, JCM 16469 | DSM 26882 | DSMZ |
| Bacteroides thetaiotaomicron-1_1_6 | HM-23 | BEI |
| Bacteroides fragilis-2_1_16 | HM-58 | BEI |
| Bacteroides xylanisolvens-2_1_22 | HM-18 | BEI |
| Parabacteroides distasonis-3_1_19 | HM-19 | BEI |
| Bacteroides dorea-9_1_42FAA | HM-27 | BEI |
| Bacteroides ovatus-D2 | HM-28 | BEI |
| Bacteroides stercoris-VPIB3-21, ATCC 43183, CIP | DSM 19555 | DSMZ |
| 104203, JCM 9496 | | |
| Bacteroides thetaiotaomicron-VPI 5482 [CIP 104206T, | ATCC 29148 | ATCC |
| E50, NCTC 10582] | | |
| Bacteroides uniformis-ATCC 8492 | ATCC 8492 | ATCC |
| Bacteroides vulgatus-NCTC 11154 | ATCC 8482 | ATCC |
| Bifidobacterium pseudocatenulatum-B1279, ATCC 27919 | DSM 20438 | DSMZ |
| Bilophila wadsworthia-WAL 7959 [Lab 88-130H] | ATCC 49260 | ATCC |
| Blautia hansenii-VPI C7-24 | DSM 20583 | DSMZ |
| Blautia hydrogenotrophica-S5a33 | DSM 10507 | DSMZ |
| Blautia obeum-ATCC 29174, KCTC 15206, VPIB3-21 | DSMZ 25238 | DSMZ |
| Blautia sp.-KLE 1732 | HM-1032 | BEI |
| Blautia wexlerae-ATCC BAA-1564, JCM 17041, KCTC | DSM 19850 | DSMZ |
| 5965, WAL 14507 | | |
| Catenibacterium mitsuokai-RCA14-39, CIP 106738, | DSM 15897 | DSMZ |
| JCM 10609 | | |
| Clostridium asparagiforme-N6, CCUG 48471 | DSM 15981 | DSMZ |
| Clostridium hylemonae-TN-271, JCM 10539 | DSM 15053 | DSMZ |
| Clostridium leptum-VPI T7-24-1, ATCC 29065 | DSM 753 | DSMZ |
| Tyzzerella nexilis DSM 1787 | DSM 1787 | DSMZ |
| Clostridium saccharolyticum-WM1, ATCC 35040, | DSM 2544 | DSMZ |
| NRC 2533 | | |
| Absiella dolichum DSM 3991 | DSM 3991 | DSMZ |
| Collinsella aerofaciens-VPI 1003 [DSM 3979, | ATCC 25986 | ATCC |
| JCM 10188] | | |
| Collinsella stercoris-RCA 55-54, JCM 10641 | DSM 13279 | DSMZ |
| Coprococcus comes-VPI CI-38 | ATCC 27758 | ATCC |
| Dialister invisus-E7.25, CCUG 47026 | DSM 15470 | DSMZ |
| Eubacterium rectale-VPI 0990 [CIP 105953] | ATCC 33656 | ATCC |
| Eubacterium siraeum-VPI T9-50-2, ATCC 29066, | DSM 15702 | DSMZ |
| DSM 3996 | | |
| Eubacterium ventriosum-VPI 1013B | ATCC 27560 | ATCC |
| Coprococcus eutactus-VPI C33-22 | ATCC 27759 | ATCC |
| Holdemanella biformis-VPI C17-5, ATCC 27806, | DSM 3989 | DSMZ |
| KCTC 5969 | | |
| Intestinibacter bartlettii-WAL 16138, ATCC BAA-827, | DSM 16795 | DSMZ |
| CCUG 48940 | | |
| Megasphaera sp.-Sanger 24, Sanger_24 | DSM 102144 | DSMZ |
| Odoribacter splanchnicus-1651/6, ATCC 29572, CCUG | DSM 20712 | DSMZ |
| 21054, CIP 104287, LMG 8202, NCTC 10825 | | |
| Parabacteroides distasonis-NCTC 11152 | ATCC 8503 | ATCC |
| Parabacteroides merdae-VPI T4-1, ATCC 43184, CCUG | DSM 19495 | DSMZ |
| 38734, CIP 104202, JCM 9497 | | |
| Parabacteroides sp.-D13 | HM-77 | BEI |
| Granulicatella adiacens-GaD [CIP 103243, DSM 9848] | ATCC 49175 | ATCC |
| Holdemania filiformis-VPI J1-31B-1, ATCC 51649 | DSM 12042 | DSMZ |
| Hungatella hathewayi-1313, CCUG 43506, CIP 109440, | DSM 13479 | DSMZ |
| MTCC 10951 | | |
| Intestinimonas butyriciproducens-SRB-521-5-1, | DSM 26588 | DSMZ |
| CCUG 63529 | | |
| Solobacterium moorei-RCA59-74, CIP 106864, | DSM 22971 | DSMZ |
| JCM 10645 | | |
| Mitsuokella multacida-A 405-1, ATCC 27723, | DSM 20544 | DSMZ |
| NCTC 10934 | | |
| Olsenella uli-D76D-27C, ATCC 49627, CIP 109912 | DSM 7084 | DSMZ |
| Parabacteroides johnsonii-M-165, CIP 109537, | DSM 18315 | DSMZ |
| JCM 13406 | | |
| Prevotella buccalis-HS4, ATCC 35310, NCDO 2354 | DSM 20616 | DSMZ |
| Prevotella copri-CB7, JCM 13464 | DSM 18205 | DSMZ |
| Roseburia inulinivorans-A2-194, CIP 109405, JCM | DSM 16841 | DSMZ |
| 17584, NCIMB 14030 | | |
| Clostridium sp.-VPIC48-50 (unassigned Clostridiales) | ATCC 29733 | ATCC |
| Ruminococcus gauvreauii-CCRI-16110, CCUG 54292, | DSM 19829 | DSMZ |
| JCM 14987, NML 060141 | | |
| Ruminococcus lactaris-VPI X6-29 | ATCC 29176 | ATCC |
| Ruminococcus torques-VPI B2-51 | ATCC 27756 | ATCC |
| Alistipes putredinis-CCUG 45780, CIP 104286, ATCC | DSM 17216 | DSMZ |
| 29800, Carlier 10203, VPI 3293 | | |
| Alistipes senegalensis-CSURP150, JCM 32779, JC50 | DSM 25460 | DSMZ |
| Clostridium spiroforme-VPI C28-23-1A, ATCC 29900, | DSM 1552 | DSMZ |
| NCTC 11211 | | |
| Slackia exigua-S-7, ATCC 700122, JCM 11022, | DSM 15923 | DSMZ |
| KCTC 5966 | | |
| Bacteroides pectinophilus-N3 | ATCC 43243 | ATCC |
| Butyrivibrio crossotus-T9-40A, ATCC 29175 | DSM 2876 | DSMZ |
| Subdoligranulum variabile-BI-114, CCUG 47106 | DSM 15176 | DSMZ |
| Turicibacter sanguinis-MOL361, NCCB 100008 | DSM 14220 | DSMZ |
| Bifidobacterium breve-S1, ATCC 15700, NCTC 11815 | DSM 20213 | DSMZ |
| Bifidobacterium catenulatum-B669, ATCC 27539, CECT | DSM 16992 | DSMZ |
| 7362, CIP 104175, DSM 20103 | | |
| Butyricimonas virosa-MT12, CCUG 56611, JCM 15149 | DSM 23226 | DSMZ |
| Streptococcus salivarius subsp. thermophilus-LMD-9 | ATCC | ATCC |
| | BAA-491 | |
| Dorea formicigenerans-VPIC8-13 [JCM 9500] | ATCC 27755 | ATCC |
| Bacteroides plebeius-M12 | DSM 17135 | DSMZ |
| Ruminococcus gnavus-VPI C7-9 | ATCC 29149 | ATCC |
| Oscillibacter sp.-KLE 1728 | HM-1030 | BEI |
| Clostridium sp.-M62/1 | HM-635 | BEI |
| Slackia heliotrinireducens-RHS 1, ATCC 29202, | DSM 20476 | DSMZ |
| NCTC 11029 | | |
| Desulfovibrio piger-VPI C3-23 [DSM 749] | ATCC 29098 | ATCC |
| Clostridium methylpentosum-R2, ATCC 43829 | DSM 5476 | DSMZ |
| Ethanoligenens harbinense-YUAN-3, CGMCC 1.5033, | DSM 18485 | DSMZ |
| JCM 12961 | | |
| Marvinbryantia formatexigens-I-52, CCUG 46960 | DSM 14469 | DSMZ |
| Lactobacillus ruminis-E 194e | ATCC 25644 | ATCC |
| Clostridium bolteae-WAL 16351, [CCUG 46953], ATCC | DSM 15670 | DSMZ |
| BAA-613, Song et al. 2003 | | |
| Clostridium hiranonis-TO-931, JCM 10541, KCTC 15199 | DSM 13275 | DSMZ |
| Clostridium scindens-VPI 13733, ATCC 35704, 19 | DSM 5676 | DSMZ |
| Bacteroides xylanisolvens-XBIA, CCUG 53782 | DSM 18836 | DSMZ |
| Clostridium sp.-L2-50 | HM-634 | BEI |
| Clostridium orbiscindens-1_3_50AFAA | HM-303 | BEI |
| Alistipes shahii-WAL 8301 | DSM 19121 | DSMZ |
| Faecalibacterium prausnitzii-A2-165, JCM 31915 | DSM 17677 | DSMZ |

In some embodiments, methods of producing a high-complexity defined gut microbial community comprise individually culturing each of a plurality of defined microbial strains prior to combining the defined microbial strains. In other embodiments, methods of producing a high-complexity defined gut microbial community comprise culturing all of a plurality of defined microbial strains together. In still other embodiments, methods of producing a high-complexity defined gut microbial community comprise individually culturing one or more defined microbial strains and culturing two or more defined microbial strains, then combining together the individually-cultured defined microbial strains and co-cultured defined microbial strains.

### 7.1 Pathway-Based Selection of High-Complexity Defined Gut Microbial Communities

The taxonomic structure of the human gut microbiome is highly variable between individuals, but the functional structure is highly conserved and informs a heuristic for the design of a metabolically comprehensive high-complexity defined gut microbial community. High-complexity defined gut microbial communities disclosed herein contain core functional diversity present in the gut microbiomes of healthy human subjects. In some embodiments high-complexity defined gut microbial communities incorporate metabolic redundancy amongst the constituent defined microbial strains to allow engraftment of the defined gut microbial community independent of the diet or genetics of the subject to which the defined gut microbial community is administered.

In some embodiments, high-complexity defined gut microbial communities disclosed herein are assembled based on the metabolic pathways utilized by one or more of the defined microbial strains rather than selecting microbial strains based on their specific taxa. In some embodiments, function/pathway-based assembly of high-complexity defined gut microbial communities is achieved by screening genomes of microbes found in donor fecal samples for the presence of: (i) core metabolic pathways of the normal human gut microbiome; and (ii) metabolic pathways involved in the consumption/metabolization of a comprehensive panel of substrates or nutrients, and/or the synthesis/production of a comprehensive panel of metabolites.

As used herein, “core metabolic pathways” refer to complete MetaCyc pathways (Caspi et al. 2018, “The MetaCyc database of metabolic pathways and enzymes”, Nucleic Acids Research 46(D1):D633-D639; MetaCyc: MetaCyc Metabolic Pathway Database [database online] [accessed May 20, 2020]. Retrieved from <https://metacyc.org/>.) that are found in the majority of gut metagenomes annotated in the GutCyc project (Hahn, Altman, Konwar, et al. GutCyc: a Multi-Study Collection of Human Gut Microbiome Metabolic Models bioRxiv. (2016); GutCyc: Collection of Pathway/Genome Dabases from the Human Gut [database online] [accessed May 20, 2020]. Retrieved from <http://gutcyc.org/>.). For example, in some embodiments, “core metabolic pathways” can be pathways where all enzymes encoding all reactions of the pathway are present in the majority of gut metagenomes annotated in the GutCyc project. Metagenomes surveyed in the GutCyc project are derived from 418 healthy human subjects from three large-scale studies (MetaHit, The Human Microbiome Project (Lloyd-Price J, Mahurkar A, Rahnavard G, et al. Strains, functions and dynamics in the expanded Human Microbiome Project. Nature. 2017; 550(7674):61-66.), and the Beijing Genomics Institute Diabetes Study Junjie Qin, Ruiqiang Li, Jeroen Raes, et al. A human gut microbial gene catalogue established by metagenomic sequencing. Nature. 2010 Mar. 4; 464(7285):59-65.)).

In some embodiments, core metabolic pathways can include any one or more of the 1CMET2-PWY, 2.6.1.32-RXN, AEROBACTINSYN-PWY, ALACAT2-PWY, ALADEG-PWY, ALANINE-DEG3-PWY, ALANINE-SYN2-PWY, ALANINE-VALINESYN-PWY, ANAPHENOXI-PWY, ARGASEDEG-PWY, ARGDEG-III-PWY, ARGDEG-IV-PWY, ARGDEGRAD-PWY, ARGDEG-V-PWY, ARG-GLU-PWY, ARGININE-SYN4-PWY, ARG-PRO-PWY, ARGSYNBSUB-PWY, ARGSYN-PWY, ASPARAGINE-BIOSYNTHESIS, ASPARAGINE-DEG1-PWY, ASPARAGINE-DEG1-PWY-1, ASPARAGINESYN-PWY, ASPARTATE-DEG1-PWY, ASPARTATESYN-PWY, ASPASN-PWY, ASPSYNII-PWY, AST-PWY, BETA-ALA-DEGRADATION-I-PWY, CAMALEXIN-SYN, CITRULBIO-PWY, CITRULLINE-DEG-PWY, COA-PWY, CODH-PWY, CYSTEINE-DEG-PWY, CYSTSYN-PWY, DAPLYSINESYN-PWY, ENTBACSYN-PWY, ETHYL-PWY, FAO-PWY, FERMENTATION-PWY, GLNSYN-PWY, GLUDEG-I-PWY, GLUGLNSYN-PWY, GLUTAMATE-DEG1-PWY, GLUTAMATE-SYN2-PWY, GLUTAMINDEG-PWY, GLUTAMINEFUM-PWY, GLUTATHIONESYN-PWY, GLUTDEG-PWY, GLUTORN-PWY, GLUTSYNIII-PWY, GLUTSYN-PWY, GLYCGREAT-PWY, GLYSYN-ALA-PWY, GLYSYN-PWY, GLYSYN-THR-PWY, HISDEG-PWY, HISHP-PWY, HISTDEG-PWY, HISTSYN-PWY, HOMOCYSDEGR-PWY, HOMOSER-METSYN-PWY, HOMOSERSYN-PWY, HSERMETANA-PWY, HYDROXYPRODEG-PWY, ILEUDEG-PWY, ILEUSYN-PWY, LARABITOLUTIL-PWY, LCYSDEG-PWY, LEU-DEG2-PWY, LEUSYN-PWY, LYSDEGII-PWY, LYSINE-AMINOAD-PWY, LYSINE-DEG1-PWY, MALATE-ASPARTATE-SHUTTLE-PWY, METH-ACETATE-PWY, METHANOGENESIS-PWY, METHIONINE-DEG1-PWY, MGLDLCTANA-PWY, ORN-AMINOPENTANOATE-CAT-PWY, ORNDEG-PWY, P101-PWY, P162-PWY, P163-PWY, P181-PWY, P261-PWY, P283-PWY, P401-PWY, P541-PWY, PHENYLALANINE-DEG1-PWY, PHESYN, PHOSLIPSYN2-PWY, PHOSPHONOTASE-PWY, PROSYN-PWY, PROUT-PWY, PWY0-1021, PWY0-1221, PWY0-1299, PWY0-1303, PWY0-1305, PWY0-1313, PWY0-1317, PWY0-1321, PWY0-1338, PWY0-1347, PWY0-1355, PWY0-1356, PWY0-1534, PWY0-1544, PWY0-1565, PWY0-1576, PWY0-1577, PWY0-1578, PWY0-1585, PWY0-1601, PWY0-42, PWY0-461, PWY0-823, PWY0-901, PWY-1, PWY-1061, PWY-1121, PWY-1186, PWY1-2, PWY-1263, PWY-1622, PWY-1722, PWY-1781, PWY-181, PWY-1881, PWY-1962, PWY-1981, PWY1F-467, PWY1F-FLAVSYN, PWY1G-0, PWY-2021, PWY-2161, PWY-2181, PWY-2201, PWY-2821, PWY-2941, PWY-2942, PWY-3, PWY-3022, PWY-3081, PWY-3161, PWY-3162, PWY-3181, PWY-3301, PWY-3341, PWY-3385, PWY-3461, PWY-3462, PWY-3581, PWY-361, PWY-3661, PWY-3661-1, PWY-381, PWY-3841, PWY-3941, PWY3DJ-12, PWY3O-4107, PWY3O-4108, PWY-4, PWY-40, PWY-4002, PWY-4041, PWY-4201, PWY-4281, PWY-43, PWY-4321, PWY-4341, PWY-4361, PWY-46, PWY490-3, PWY490-4, PWY-4981, PWY-4983, PWY-4984, PWY4FS-6, PWY-5, PWY-5022, PWY-5024, PWY-5028, PWY-5029, PWY-5030, PWY-5031, PWY-5041, PWY-5048, PWY-5049, PWY-5057, PWY-5075, PWY-5076, PWY-5078, PWY-5079, PWY-5081, PWY-5082, PWY-5087, PWY-5097, PWY-5101, PWY-5103, PWY-5104, PWY-5108, PWY-5109, PWY-5129, PWY-5135, PWY-5136, PWY-5151, PWY-5154, PWY-5155, PWY-5159, PWY-5176, PWY-5188, PWY-5189, PWY-5196, PWY-5199, PWY-5207, PWY-5250, PWY-5254, PWY-5265, PWY-5280, PWY-5283, PWY-5290, PWY-5297, PWY-5298, PWY-5311, PWY-5314, PWY-5316, PWY-5319, PWY-5324, PWY-5329, PWY-5331, PWY-5332, PWY-5364, PWY-5381, PWY-5382, PWY-5386, PWY-5394, PWY-5399, PWY-5436, PWY-5437, PWY-5441, PWY-5443, PWY-5458, PWY-5467, PWY-5468, PWY-5473, PWY-5474, PWY-5494, PWY-5497, PWY-5499, PWY-5629, PWY-5651, PWY-5653, PWY-5665, PWY-5669, PWY-5675, PWY-5679, PWY-5686, PWY-5710, PWY-5736, PWY-5737, PWY-5739, PWY-5740, PWY-5742, PWY-5747, PWY-5748, PWY-5751, PWY-5754, PWY-5766, PWY-5770, PWY-5784, PWY-5788, PWY-5797, PWY-5800, PWY-581, PWY-5811, PWY-5818, PWY-5826, PWY-5877, PWY-5883, PWY-5886, PWY-5912, PWY-5913, PWY-5921, PWY-5936, PWY-5940, PWY-5958, PWY-5963, PWY-5968, PWY-5978, PWY-5980, PWY-5990, PWY-6003, PWY-6004, PWY-601, PWY-6030, PWY-6039, PWY-6045, PWY-6052, PWY-6053, PWY-6054, PWY-6055, PWY-6068, PWY-6069, PWY-6082, PWY-6120, PWY-6121, PWY-6122, PWY-6123, PWY-6124, PWY-6133, PWY-6134, PWY-6141, PWY-6143, PWY-6148, PWY-6151, PWY-6160, PWY-6173, PWY-6196, PWY-6219, PWY-622, PWY-6220, PWY-6233, PWY-6273, PWY-6277, PWY-6281, PWY-6307, PWY-6309, PWY-6313, PWY-6318, PWY-6320, PWY-6321, PWY-6322, PWY-6324, PWY-6328, PWY-6334, PWY-6339, PWY-6343, PWY-6344, PWY-6345, PWY-6346, PWY-6375, PWY-6376, PWY-6381, PWY-6386, PWY-6387, PWY-6397, PWY-6403, PWY-6407, PWY-6408, PWY-6409, PWY-6431, PWY-6435, PWY-6444, PWY-6455, PWY-6456, PWY-6457, PWY-6466, PWY-6471, PWY-6473, PWY-6481, PWY-6486, PWY-6493, PWY-6495, PWY-6511, PWY-6512, PWY-6519, PWY-6533, PWY-6535, PWY-6536, PWY-6537, PWY-6543, PWY-6549, PWY-6559, PWY-6562, PWY-6572, PWY-6573, PWY-6574, PWY-6578, PWY-6588, PWY-6614, PWY-6627, PWY66-301, PWY66-375, PWY-6638, PWY66-391, PWY-6642, PWY66-420, PWY66-421, PWY66-425, PWY66-426, PWY66-428, PWY-6643, PWY-6661, PWY-6673, PWY-6682, PWY-6690, PWY-6696, PWY-6711, PWY-6717, PWY-6720, PWY-6724, PWY-6728, PWY-6731, PWY-6735, PWY-6749, PWY-6769, PWY-6771, PWY-6772, PWY-6773, PWY-6781, PWY-6784, PWY-6790, PWY-6791, PWY-6802, PWY-6807, PWY-6808, PWY-6813, PWY-6815, PWY-6816, PWY-6817, PWY-6818, PWY-6821, PWY-6822, PWY-6823, PWY-6831, PWY-6832, PWY-6834, PWY-6840, PWY-6845, PWY-6853, PWY-6854, PWY-6855, PWY-6891, PWY-6892, PWY-6896, PWY-6898, PWY-6902, PWY-6907, PWY-6908, PWY-6920, PWY-6922, PWY-6936, PWY-6942, PWY-6949, PWY-6953, PWY-6955, PWY-6963, PWY-6964, PWY-6965, PWY-6968, PWY-6969, PWY-6981, PWY-6982, PWY-6986, PWY-6994, PWY-7000, PWY-701, PWY-7014, PWY-7015, PWY-7016, PWY-7018, PWY-7019, PWY-702, PWY-7022, PWY-7025, PWY-7028, PWY-7040, PWY-7046, PWY-7052, PWY-7054, PWY-7064, PWY-7072, PWY-7088, PWY-7090, PWY-7097, PWY-7104, PWY-7115, PWY-7117, PWY-7118, PWY-7126, PWY-7147, PWY-7153, PWY-7158, PWY-7176, PWY-7177, PWY-7185, PWY-7186, PWY-7219, PWY-7221, PWY-7234, PWY-7246, PWY-7248, PWY-7250, PWY-7255, PWY-7274, PWY-7275, PWY-7282, PWY-7288, PWY-7297, PWY-7304, PWY-7315, PWY-7316, PWY-7318, PWY-7342, PWY-7351, PWY-7356, PWY-7376, PWY-7377, PWY-7383, PWY-7387, PWY-7397, PWY-7398, PWY-7400, PWY-7414, PWY-7425, PWY-7430, PWY-7432, PWY-7440, PWY-7441, PWY-7456, PWY-7467, PWY-7498, PWY-7501, PWY-7506, PWY-7510, PWY-7514, PWY-7518, PWY-7520, PWY-7525, PWY-7531, PWY-7532, PWY-7533, PWY-7536, PWY-7542, PWY-7543, PWY-7547, PWY-7549, PWY-7550, PWY-7555, PWY-7561, PWY-7565, PWY-7570, PWY-7571, PWY-7600, PWY-7605, PWY-761, PWY-7612, PWY-7626, PWY-7645, PWY-7648, PWY-7649, PWY-7650, PWY-7665, PWY-7667, PWY-7668, PWY-7669, PWY-7671, PWY-7674, PWY-7688, PWY-7690, PWY-7693, PWY-7694, PWY-7701, PWY-7704, PWY-7706, PWY-7708, PWY-7717, PWY-7718, PWY-7719, PWY-7733, PWY-7734, PWY-7735, PWY-7737, PWY-7751, PWY-7761, PWY-7765, PWY-7767, PWY-7769, PWY-7770, PWY-7782, PWY-7790, PWY-7791, PWY-7793, PWY-7797, PWY-7811, PWY-7814, PWY-7822, PWY-7824, PWY-7826, PWY-7842, PWY-7850, PWY-7851, PWY-7855, PWY-7860, PWY-7861, PWY-7863, PWY-7867, PWY-7870, PWY-7880, PWY-7888, PWY-7889, PWY-7891, PWY-7892, PWY-7897, PWY-7901, PWY-7904, PWY-7907, PWY-7909, PWY-7910, PWY-7913, PWY-7917, PWY-7930, PWY-7931, PWY-7936, PWY-7953, PWY-7955, PWY-7956, PWY-7957, PWY-7958, PWY-7959, PWY-7960, PWY-7962, PWY-7977, PWY-7985, PWY-7986, PWY-7987, PWY-7988, PWY-7990, PWY-8002, PWY-8003, PWY-8006, PWY-8007, PWY-8008, PWY-8009, PWY-801, PWY-8010, PWY-8011, PWY-8013, PWY-8014, PWY-8015, PWY-8016, PWY-8017, PWY-8024, PWY-8032, PWY-8040, PWY-8043, PWY-8045, PWY-8071, PWY-8072, PWY-8080, PWY-8081, PWY-8082, PWY-8083, PWY-8086, PWY-8088, PWY-842, PWY-861, PWY-862, PWY8J2-1, PWY8J2-22, PWYDQC-4, PWYG-321, PWY-I9, PWYQT-4450, PWYQT-4476, PYRIDNUCSAL-PWY, PYRIDNUCSYN-PWY, PYRIDOXSYN-PWY, SAM-PWY, SERDEG-PWY, SERSYN-PWY, SPHINGOLIPID-SYN-PWY, TAURINEDEG-PWY, THRDLCTCAT-PWY, THREONINE-DEG2-PWY, TRNA-CHARGING-PWY, TRPCAT-PWY, TRPIAACAT-PWY, TRPKYNCAT-PWY, TRPSYN-PWY, TRYPDEG-PWY, TYRFUMCAT-PWY, TYRSYN, UDPNACETYLGALSYN-PWY, UDPNAGSYN-PWY, VALDEG-PWY, and VALSYN-PWY MetaCyc pathways. For example, in some embodiments, core metabolic pathways can include at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or all the MetaCyc pathways described above.

The common names of the core MetaCyc pathways described above are provided in Table 9 below.

TABLE 9
Core MetaCyc Pathways

| MetaCyc Pathway ID | Pathway Common Name |
|---|---|
| 12DICHL0RETHDEG-PW Y | 1,2-dichloroethane degradation |
| 1CMET2-PWY | formylTHF biosynthesis I |
| 2OXOBUTYRATECAT-PWY | 2-oxobutanoate degradation II |
| ACETOACETATE-DEG-PWY | acetoacetate degradation (to acetyl Co A) |
| ALACAT2-PWY | alanine degradation II (to D-lactate) |
| ALANINE-SYN2-PWY | alanine biosynthesis II |
| ALANINE-VALINESYN-PWY | alanine biosynthesis I |
| ANAGLYCOLYSIS-PWY | glycolysis III (from glucose) |
| ANARESP1-PWY | respiration (anaerobic) |
| ARABCAT-PWY | L-arabinose degradation I |
| ARG-PRO-PWY | arginine degradation VI (arginase 2 pathway) |
| ARGASEDEG-PWY | arginine degradation I (arginase pathway) |
| ARGDEG-III-PWY | arginine degradation IV (arginine |
| | decarboxylase/agmatine deiminase pathway) |
| ARGDEG-V-PWY | arginine degradation X (arginine monooxygenase |
| | pathway) |
| ARGS YNB SUB-PWY | arginine biosynthesis II (acetyl cycle) |
| ASPARAGINE-BIOSYNTHESIS | asparagine biosynthesis I |
| ASPARAGINE-DEG1-PWY | asparagine degradation I |
| ASPARAGINESYN-PWY | asparagine biosynthesis II |
| AST-PWY | arginine degradation II (AST pathway) |
| BGALACT-PWY | lactose degradation III |
| BSUBPOLYAMSYN-PWY | spermidine biosynthesis I |
| CATECHOL-ORTHO-CLEAVAGE- | catechol degradation to beta-ketoadipate |
| PWY | |
| CENTBENZCOA-PWY | benzoyl-CoA degradation II (anaerobic) |
| CENTFERM-PWY | pyruvate fermentation to butanoate |
| CHLOROPHYLL-SYN | chlorophyllide a biosynthesis I (aerobic, light- |
| | dependent) |
| CITRULLINE-DEG-PWY | citrulline degradation |
| COA-PWY | coenzyme A biosynthesis |
| COBALSYN-PWY | adenosylcobalamin salvage from cobinamide I |
| CRNFORCAT-PWY | creatinine degradation I |
| CYSTSYN-PWY | cysteine biosynthesis I |
| DAPLYSINESYN-PWY | lysine biosynthesis I |
| DARABCATK12-PWY | D-arabinose degradation I |
| DENITRIFICATION-PWY | nitrate reduction I (denitrification) |
| DETOX1-PWY | superoxide radicals degradation |
| DTDPRHAMSYN-PWY | dTDP-L-rhamnose biosynthesis I |
| ETOH-ACETYLCOA-ANA-PWY | ethanol degradation I |
| FASYN-ELONG-PWY | fatty acid elongation-saturated |
| FERMENTATION-PWY | mixed acid fermentation |
| FUCCAT-PWY | fucose degradation |
| GALACTARDEG-PWY | D-galactarate degradation I |
| GALACTCAT-PWY | D-galactonate degradation |
| GALACTUROCAT-PWY | D-galacturonate degradation I |
| GLUAMCAT-PWY | N-acetylglucosamine degradation I |
| GLUCARDEG-PWY | D-glucarate degradation I |
| GLUCONEO-PWY | gluconeogenesis I |
| GLUCONSUPER-PWY | D-gluconate degradation |
| GLUCOSE1PMETAB-PWY | glucose and glucose-1-phosphate degradation |
| GLUTAMINDEG-PWY | glutamine degradation I |
| GLUTAMINEFUM-PWY | glutamine degradation II |
| GLUTATHIONESYN-PWY | glutathione biosynthesis |
| GLUTDEG-PWY | glutamate degradation II |
| GLUTORN-PWY | ornithine biosynthesis |
| GLUTSYN-PWY | glutamate biosynthesis I |
| GLUTSYNIII-PWY | glutamate biosynthesis III |
| GLYCEROLMETAB-PWY | glycerol degradation V |
| GLYCLEAV-PWY | glycine cleavage |
| GLYCOCAT-PWY | glycogen degradation I |
| GLYCOGENS YNTH-PWY | glycogen biosynthesis I (from ADP-D-Glucose) |
| GLYCOLYSIS | glycolysis I (from glucose-6P) |
| GLYSYN-PWY | glycine biosynthesis I |
| GLYSYN-THR-PWY | glycine biosynthesis IV |
| HEME-BIOSYNTHESIS-II | heme biosynthesis from uroporphyrinogen-III I |
| HEMESYN2-PWY | heme biosynthesis from uroporphyrinogen-III II |
| HISDEG-PWY | histidine degradation I |
| HISTSYN-PWY | histidine biosynthesis |
| HOMOSER-METSYN-PWY | methionine biosynthesis I |
| HOMOSER-THRESYN-PWY | threonine biosynthesis from homoserine |
| HOMOSERSYN-PWY | homoserine biosynthesis |
| ILEUDEG-PWY | isoleucine degradation I |
| ILEUSYN-PWY | isoleucine biosynthesis I (from threonine) |
| KDO-LIPASYN-PWY | (KD0)2-lipid A biosynthesis 1 |
| LACTOSECAT-PWY | lactose and galactose degradation I |
| LACTOSEUTIL-PWY | lactose degradation II |
| LARABITOLUTIL-PWY | xylitol degradation |
| LCYSDEG-PWY | L-cysteine degradation II |
| LEUSYN-PWY | leucine biosynthesis |
| LIPAS-PWY | triacylglycerol degradation |
| LIPASYN-PWY | phospholipases |
| MALTOSECAT-PWY | maltose degradation |
| MANNCAT-PWY | D-mannose degradation |
| MANNIDEG-PWY | mannitol degradation I |
| MENAQUINONESYN-PWY | menaquinol-8 biosynthesis |
| METH-ACETATE-PWY | methanogenesis from acetate |
| METHFORM-PWY | methyl-coenzyme M reduction to methane |
| METHIONINE-DEG1-PWY | methionine degradation I (to homocysteine) |
| N2FIX-PWY | nitrogen fixation |
| NAD-BIOSYNTHESIS-III | NAD biosynthesis III |
| NAGLIPASYN-PWY | lipid IVA biosynthesis |
| NONMEVIPP-PWY | methylerythritol phosphate pathway |
| NONOXIPENT-PWY | pentose phosphate pathway (non-oxidative branch) |
| NPGLUCAT-PWY | Entner-Doudoroff pathway II (non- |
| | phosphorylative) |
| OXIDATIVEPENT-PWY | pentose phosphate pathway (oxidative branch) |
| P105-PWY | TCA cycle IV (2-oxoglutarate decarboxylase) |
| P121-PWY | adenine and adenosine salvage I |
| P122-PWY | heterolactic fermentation |
| P124-PWY | Bifidobacterium shunt |
| P162-PWY | glutamate degradation V (via hydroxyglutarate) |
| Pl 63-PWY | lysine fermentation to acetate and butyrate |
| P164-PWY | purine nucleobases degradation I (anaerobic) |
| P2-PWY | 2-(5-phosphoribosyl)-3-dephospho-CoA |
| | biosynthesis I (citrate lyase) |
| P21-PWY | pentose phosphate pathway (partial) |
| P283-PWY | hydrogen oxidation I (aerobic) |
| P3-PWY | gallate degradation III (anaerobic) |
| P302-PWY | L-sorbose degradation |
| P321-PWY | benzoyl-CoA degradation III (anaerobic) |
| P42-PWY | incomplete reductive TCA cycle |
| P562-PWY | myo-inositol degradation I |
| PANTO-PWY | phosphopantothenate biosynthesis I |
| PHENYLALANINE-DEG1-PWY | phenylalanine degradation I (aerobic) |
| PHESYN | phenylalanine biosynthesis I |
| PHOSPHONOTASE-PWY | 2-aminoethylphosphonate degradation I |
| PLPSAL-PWY | pyridoxal 5-phosphate salvage I |
| PPGPPMET-PWY | ppGpp biosynthesis |
| PROPIONMET-PWY | methylmalonyl pathway |
| PROSYN-PWY | proline biosynthesis I |
| PROUT-PWY | proline degradation |
| PWY-1001 | cellulose biosynthesis |
| PWY-1081 | homogalacturonan degradation |
| PWY-1269 | CMP-KDO biosynthesis I |
| PWY-1622 | formaldehyde assimilation I (serine pathway) |
| PWY-1722 | formaldehyde oxidation V (tetrahydrofolate |
| | pathway) |
| PWY-1881 | formate oxidation to CO2 |
| PWY-2161 | folate polyglutamylation |
| PWY-2221 | Entner-Doudoroff pathway III (semi- |
| | phosphorylative) |
| PWY-2301 | myo-inositol biosynthesis |
| PWY-2361 | 3-oxoadipate degradation |
| PWY-2622 | trehalose biosynthesis IV |
| PWY-2661 | trehalose biosynthesis V |
| PWY-2941 | lysine biosynthesis II |
| PWY-2942 | lysine biosynthesis III |
| PWY-3221 | dTDP-L-rhamnose biosynthesis II |
| PWY-3561 | choline biosynthesis III |
| PWY-3781 | aerobic respiration (cytochrome c) |
| PWY-3982 | uracil degradation I (reductive) |
| PWY-4 | UDP-D-galacturonate biosynthesis II (from D- |
| | galacturonate) |
| PWY-4081 | glutathione redox reactions I |
| PWY-4101 | D-sorbitol degradation I |
| PWY-4121 | glutathionylspermidine biosynthesis |
| PWY-4261 | glycerol degradation I |
| PWY-43 | putrescine biosynthesis II |
| PWY-4341 | glutamate biosynthesis V |
| PWY-43 81 | fatty acid biosynthesis initiation I |
| PWY-4621 | arsenate detoxification II (glutaredoxin) |
| PWY-4722 | creatinine degradation II |
| PWY-4921 | protein citrullination |
| PWY-4984 | urea cycle |
| PWY-5022 | 4-aminobutyrate degradation V |
| PWY-5041 | S-adenosyl-L-methionine cycle II |
| PWY-5046 | 2-oxoisovalerate decarboxylation to isobutanoyl- |
| | CoA |
| PWY-5057 | valine degradation II |
| PWY-5084 | 2-oxoglutarate decarboxylation to succinyl-CoA |
| PWY-5097 | lysine biosynthesis VI |
| PWY-5101 | isoleucine biosynthesis II |
| PWY-5104 | isoleucine biosynthesis IV |
| PWY-5122 | geranyl diphosphate biosynthesis |
| PWY-5142 | acyl-ACP thioesterase pathway |
| PWY-5143 | fatty acid activation |
| PWY-5148 | acyl-CoA hydrolysis |
| PWY-5155 | beta-alanine biosynthesis III |
| PWY-5162 | 2-oxopentenoate degradation |
| PWY-5188 | tetrapyrrole biosynthesis I (from glutamate) |
| PWY-5194 | siroheme biosynthesis |
| PWY-5207 | coenzyme B/coenzyme M regeneration |
| PWY-5261 | methanogenesis from tetramethylammonium |
| PWY-5278 | sulfite oxidation III |
| PWY-5340 | sulfate activation for sulfonation |
| PWY-5344 | homocysteine biosynthesis |
| PWY-5350 | thiosulfate disproportionation III (rhodanese) |
| PWY-5382 | hydrogen oxidation II (aerobic, NAD) |
| PWY-5384 | sucrose degradation IV (sucrose phosphorylase) |
| PWY-5386 | methylglyoxal degradation I |
| PWY-5436 | threonine degradation IV |
| PWY-5480 | pyruvate fermentation to ethanol I |
| PWY-5481 | pyruvate fermentation to lactate |
| PWY-5493 | reductive monocarboxylic acid cycle |
| PWY-5497 | purine nucleobases degradation II (anaerobic) |
| PWY-5508 | adenosylcobalamin biosynthesis from cobyrinate |
| | a,c-diamide II |
| PWY-5509 | adenosylcobalamin biosynthesis from cobyrinate |
| | a,c-diamide I |
| PWY-5659 | GDP-mannose biosynthesis |
| PWY-5667 | CDP-diacylglycerol biosynthesis I |
| PWY-5668 | cardiolipin biosynthesis I |
| PWY-5669 | phosphatidylethanolamine biosynthesis I |
| PWY-5674 | nitrate reduction IV (dissimilatory) |
| PWY-5677 | succinate fermentation to butyrate |
| PWY-5686 | UMP biosynthesis |
| PWY-5695 | urate biosynthesis/inosine 5-phosphate degradation |
| PWY-5698 | allantoin degradation to ureidoglycolate II |
| | (ammonia producing) |
| PWY-5703 | urea degradation I |
| PWY-5704 | urea degradation II |
| PWY-5743 | 3-hydroxypropionate cycle |
| PWY-5751 | phenylethanol biosynthesis |
| PWY-5783 | octaprenyl diphosphate biosynthesis |
| PWY-5785 | di-trans,poly-cis-undecaprenyl phosphate |
| | biosynthesis |
| PWY-5789 | 3-hydroxypropionate/4-hydroxybutyrate cycle |
| PWY-5791 | 1,4-dihydroxy-2-naphthoate biosynthesis II |
| | (plants) |
| PWY-5794 | malonate degradation I (biotin-independent) |
| PWY-5807 | heptaprenyl diphosphate biosynthesis |
| PWY-5831 | CDP-abequose biosynthesis |
| PWY-5833 | CDP-3,6-dideoxyhexose biosynthesis |
| PWY-5837 | 1,4-dihydroxy-2-naphthoate biosynthesis I |
| PWY-5839 | menaquinol-7 biosynthesis |
| PWY-5844 | menaquinol-9 biosynthesis |
| PWY-5849 | menaquinol-6 biosynthesis |
| PWY-5851 | demethylmenaquinol-9 biosynthesis |
| PWY-5852 | demethylmenaquinol-8 biosynthesis I |
| PWY-5853 | demethylmenaquinol-6 biosynthesis |
| PWY-5875 | staphyloxanthin biosynthesis |
| PWY-5886 | 4-hydroxyphenylpyruvate biosynthesis |
| PWY-5890 | menaquinol-10 biosynthesis |
| PWY-5891 | menaquinol-11 biosynthesis |
| PWY-5892 | menaquinol-12 biosynthesis |
| PWY-5895 | menaquinol-13 biosynthesis |
| PWY-5901 | 2,3-dihydroxybenzoate biosynthesis |
| PWY-5913 | TCA cycle VI (obligate autotrophs) |
| PWY-5921 | L-glutamine biosynthesis II (tRNA-dependent) |
| PWY-5940 | streptomycin biosynthesis |
| PWY-5941 | glycogen degradation II |
| PWY-5964 | guanylyl molybdenum cofactor biosynthesis |
| PWY-5971 | palmitate biosynthesis II (bacteria and plants) |
| PWY-5973 | cis-vaccenate biosynthesis |
| PWY-5988 | wound-induced proteolysis I |
| PWY-5989 | stearate biosynthesis II (bacteria and plants) |
| PWY-6012 | acyl carrier protein metabolism |
| PWY-6018 | seed germination protein turnover |
| PWY-6019 | pseudouridine degradation |
| PWY-6028 | acetoin degradation |
| PWY-6038 | citrate degradation |
| PWY-6121 | 5-aminoimidazole ribonucleotide biosynthesis I |
| PWY-6122 | 5-aminoimidazole ribonucleotide biosynthesis II |
| PWY-6131 | glycerol degradation II |
| PWY-6139 | CMP-N-acetylneuraminate biosynthesis II |
| | (bacteria) |
| PWY-6143 | CMP-pseudaminate biosynthesis |
| PWY-6147 | 6-hydroxymethyl-dihydropterin diphosphate |
| | biosynthesis I |
| PWY-6153 | autoinducer AI-2 biosynthesis I |
| PWY-6154 | autoinducer AI-2 biosynthesis II (Vibrio) |
| PWY-6163 | chorismate biosynthesis from 3-dehydroquinate |
| PWY-6164 | 3-dehydroquinate biosynthesis I |
| PWY-6173 | histamine biosynthesis |
| PWY-6193 | 3-chlorocatechol degradation II (ortho) |
| PWY-6196 | serine racemization |
| PWY-621 | sucrose degradation III (sucrose invertase) |
| PWY-622 | starch biosynthesis |
| PWY-6268 | adenosylcobalamin salvage from cobalamin |
| PWY-6269 | adenosylcobalamin salvage from cobinamide II |
| PWY-6282 | palmitoleate biosynthesis I |
| PWY-6317 | galactose degradation I (Leloir pathway) |
| PWY-6322 | phosphinothricin tripeptide biosynthesis |
| PWY-6344 | ornithine degradation II (Stickland reaction) |
| PWY-6348 | phosphate acquisition |
| PWY-6349 | CDP-archaeol biosynthesis |
| PWY-6357 | phosphate utilization in cell wall regeneration |
| PWY-6386 | UDP-N-acetylmuramoyl-pentapeptide |
| | biosynthesis II (lysine-containing) |
| PWY-6387 | UDP-N-acetylmuramoyl-pentapeptide |
| | biosynthesis III (meso-DAP-containing) |
| PWY-6397 | mycolyl-arabinogalactan-peptidoglycan complex |
| | biosynthesis |
| PWY-6430 | thymine degradation |
| PWY-6461 | peptidoglycan cross-bridge biosynthesis II (E. |
| | faecium) |
| PWY-6465 | omega-hydroxylation of caprate and laurate |
| PWY-6476 | cytidylyl molybdenum cofactor biosynthesis |
| PWY-6507 | 5-dehydro-4-deoxy-D-glucuronate degradation |
| PWY-6512 | hydrogen oxidation III (anaerobic, NADP) |
| PWY-6518 | glycocholate metabolism (bacteria) |
| PWY-6519 | 8-amino-7-oxononanoate biosynthesis I |
| PWY-6543 | 4-aminobenzoate biosynthesis |
| PWY-6545 | pyrimidine deoxyribonucleotides de novo |
| | biosynthesis III |
| PWY-6559 | spermidine biosynthesis II |
| PWY-6562 | norspermidine biosynthesis |
| PWY-6572 | chondroitin sulfate and dermatan sulfate |
| | degradation I (bacterial) |
| PWY-6578 | 8-amino-7-oxononanoate biosynthesis III |
| PWY-6583 | pyruvate fermentation to butanol I |
| PWY-6587 | pyruvate fermentation to ethanol III |
| PWY-6599 | guanine and guanosine salvage II |
| PWY-66 | GDP-L-fucose biosynthesis I (from GDP-D- |
| | mannose) |
| PWY-6608 | guanosine nucleotides degradation III |
| PWY-6609 | adenine and adenosine salvage III |
| PWY-6610 | adenine and adenosine salvage IV |
| PWY-6613 | tetrahydrofolate salvage from 5,10- |
| | methenyltetrahydrofolate |
| PWY-6614 | tetrahydrofolate biosynthesis |
| PWY-6617 | adenosine nucleotides degradation III |
| PWY-6620 | guanine and guanosine salvage I |
| PWY-6627 | salinosporamide A biosynthesis |
| PWY-6638 | sulfolactate degradation III |
| PWY-6642 | (R)-cysteate degradation |
| PWY-6643 | coenzyme M biosynthesis II |
| PWY-6649 | glycolate and glyoxylate degradation III |
| PWY-6695 | oxalate degradation II |
| PWY-6700 | queuosine biosynthesis |
| PWY-6703 | preQ0 biosynthesis |
| PWY-6708 | ubiquinol-8 biosynthesis (prokaryotic) |
| PWY-6737 | starch degradation V |
| PWY-6744 | hydrogen production I |
| PWY-6756 | S-methyl-5-thioadenosine degradation II |
| PWY-6758 | hydrogen production II |
| PWY-6769 | rhamnogalacturonan type I degradation I (fungi) |
| PWY-6772 | hydrogen production V |
| PWY-6780 | hydrogen production VI |
| PWY-6785 | hydrogen production VIII |
| PWY-6815 | porphyran degradation |
| PWY-6816 | agarose degradation |
| PWY-6823 | molybdenum cofactor biosynthesis |
| PWY-6827 | gellan degradation |
| PWY-6855 | chitin degradation I (archaea) |
| PWY-6890 | 4-amino-2-methyl-5-diphosphomethylpyrimidine |
| | biosynthesis |
| PWY-6892 | thiazole biosynthesis I (E. coli) |
| PWY-6893 | thiamin diphosphate biosynthesis II (Bacillus) |
| PWY-6894 | thiamin diphosphate biosynthesis I (E. coli) |
| PWY-6896 | thiamin salvage I |
| PWY-6898 | thiamin salvage III |
| PWY-6899 | base-degraded thiamin salvage |
| PWY-6902 | chitin degradation II |
| PWY-6906 | chitin derivatives degradation |
| PWY-6907 | thiamin diphosphate biosynthesis III |
| | (Staphylococcus) |
| PWY-6910 | hydroxymethylpyrimidine salvage |
| PWY-6932 | selenate reduction |
| PWY-6938 | NADH repair |
| PWY-6943 | testosterone and androsterone degradation to |
| | androstendione |
| PWY-6944 | androstenedione degradation |
| PWY-6946 | cholesterol degradation to androstenedione II |
| | (cholesterol dehydrogenase) |
| PWY-6948 | sitosterol degradation to androstenedione |
| PWY-6951 | docosahexanoate biosynthesis II |
| PWY-6952 | glycerophosphodiester degradation |
| PWY-6961 | L-ascorbate degradation II (bacteria1, aerobic) |
| PWY-6964 | ammonia assimilation cycle II |
| PWY-6969 | TCA cycle V (2-oxoglutarate:ferredoxin |
| | oxidoreductase) |
| PWY-6984 | lipoate salvage II |
| PWY-6986 | alginate degradation |
| PWY-6987 | lipoate biosynthesis and incorporation III |
| | (Bacillus) |
| PWY-6999 | theophylline degradation |
| PWY-701 | methionine degradation II |
| PWY-7028 | UDP-N,N-diacetylbacillosamine biosynthesis |
| PWY-7054 | N-acetylglutaminylglutamine amide biosynthesis |
| PWY-7096 | triclosan resistance |
| PWY-7159 | chlorophyllide a biosynthesis III (aerobic, light |
| | independent) |
| PWY-7174 | S-methyl-5-thio-alpha-D-ribose 1-phosphate |
| | degradation II |
| PWY-7176 | UTP and CTP de novo biosynthesis |
| PWY-7179 | purine deoxyribonucleosides degradation I |
| PWY-7180 | 2-deoxy-alpha-D-ribose 1-phosphate degradation |
| PWY-7181 | pyrimidine deoxyribonucleosides degradation |
| PWY-7183 | pyrimidine nucleobases salvage I |
| PWY-7184 | pyrimidine deoxyribonucleotides de novo |
| | biosynthesis I |
| PWY-7185 | UTP and CTP dephosphorylation I |
| PWY-7187 | pyrimidine deoxyribonucleotides de novo |
| | biosynthesis II |
| PWY-7193 | pyrimidine ribonucleosides salvage I |
| PWY-7197 | pyrimidine deoxyribonucleotide phosphorylation |
| PWY-7199 | pyrimidine deoxyribonucleosides salvage |
| PWY-7205 | CMP phosphorylation |
| PWY-7210 | pyrimidine deoxyribonucleotides biosynthesis |
| | from CTP |
| PWY-7219 | adenosine ribonucleotides de novo biosynthesis |
| PWY-7220 | adenosine deoxyribonucleotides de novo |
| | biosynthesis II |
| PWY-7221 | guanosine ribonucleotides de novo biosynthesis |
| PWY-7222 | guanosine deoxyribonucleotides de novo |
| | biosynthesis II |
| PWY-7224 | purine deoxyribonucleosides salvage |
| PWY-7242 | D-fructuronate degradation |
| PWY-7246 | pectin degradation II |
| PWY-7247 | beta-D-glucuronide and D-glucuronate |
| | degradation |
| PWY-7248 | pectin degradation III |
| PWY-7250 | [2Fe-2S] iron-sulfur cluster biosynthesis |
| PWY-7285 | methylwyosine biosynthesis |
| PWY-7286 | 7-(3-amino-3-carboxypropyl)-wy osine |
| | biosynthesis |
| PWY-7294 | xylose degradation IV |
| PWY-7295 | L-arabinose degradation IV |
| PWY-7308 | acrylonitrile degradation I |
| PWY-7330 | UDP-N-acetyl-beta-L-fucosamine biosynthesis |
| PWY-7331 | UDP-N-acetyl-beta-L-quinovosamine biosynthesis |
| PWY-7333 | UDP-N-acetyl-alpha-D-fucosamine biosynthesis |
| PWY-7334 | UDP-N-acetyl-alpha-D-quinovosamine |
| | biosynthesis |
| PWY-7335 | UDP-N-acetyl-alpha-D-mannosaminouronate |
| | biosynthesis |
| PWY-7343 | UDP-glucose biosynthesis |
| PWY-7344 | UDP-D-galactose biosynthesis |
| PWY-7346 | UDP-alpha-D-glucuronate biosynthesis (from |
| | UDP-glucose) |
| PWY-7347 | sucrose biosynthesis III |
| PWY-7353 | 4-methyl-5(beta-hydroxyethyl)thiazole salvage |
| | (yeast) |
| PWY-7356 | thiamin salvage IV (yeast) |
| PWY-7367 | phosphatidylcholine resynthesis via |
| | glycerophosphocholine |
| PWY-901 | methylglyoxal degradation II |
| PWY0-1021 | alanine biosynthesis III |
| PWY0-1182 | trehalose degradation II (trehalase) |
| PWY0-1241 | ADP-L-glycero-beta-D-manno-heptose |
| | biosynthesis |
| PWY0-1261 | 1,6-anhydro-N-acetylmuramic acid recycling |
| PWY0-1264 | biotin-carboxyl carrier protein assembly |
| PWY0-1275 | lipoate biosynthesis and incorporation II |
| PWY0-1280 | ethylene glycol degradation |
| PWY0-1295 | pyrimidine ribonucleosides degradation |
| PWY0-1296 | purine ribonucleosides degradation |
| PWY0-1299 | arginine dependent acid resistance |
| PWY0-1300 | 2-O-alpha-mannosyl-D-glycerate degradation |
| PWY0-1301 | melibiose degradation |
| PWY0-1305 | glutamate dependent acid resistance |
| PWY0-1312 | acetate formation from acetyl-CoA I |
| PWY0-1313 | acetate conversion to acetyl-CoA |
| PWY0-1314 | fructose degradation |
| PWY0-1315 | L-lactaldehyde degradation (anaerobic) |
| PWY0-1317 | L-lactaldehyde degradation (aerobic) |
| PWY0-1319 | CDP-diacylglycerol biosynthesis II |
| PWY0-1324 | N-acetylneuraminate and N-acetylmannosamine |
| | degradation |
| PWY0-1334 | NADH to cytochrome bd oxidase electron transfer |
| PWY0-1338 | polymyxin resistance |
| PWY0-1353 | succinate to cytochrome bd oxidase electron |
| | transfer |
| PWY0-1477 | ethanolamine utilization |
| PWY0-1479 | tRNA processing |
| PWY0-1507 | biotin biosynthesis from 8-amino-7-oxononanoate |
| PWY0-1535 | D-serine degradation |
| PWY0-1545 | cardiolipin biosynthesis III |
| PWY0-1546 | muropeptide degradation |
| PWY0-321 | phenylacetate degradation I (aerobic) |
| PWY0-42 | 2-methylcitrate cycle I |
| PWY0-43 | conversion of succinate to propionate |
| PWY0-461 | lysine degradation I |
| PWY0-501 | lipoate biosynthesis and incorporation I |
| PWY0-522 | lipoate salvage I |
| PWY0-541 | cyclopropane fatty acid (CFA) biosynthesis |
| PWY0-662 | PRPP biosynthesis I |
| PWY0-823 | arginine degradation III (arginine |
| | decarboxylase/agmatinase pathway) |
| PWY0-862 | cis-dodecenoyl biosynthesis |
| PWY0-901 | selenocysteine biosynthesis I (bacteria) |
| PWY1-2 | alanine degradation IV |
| PWY1A0-6325 | actinorhodin biosynthesis |
| PWY3O-4106 | NAD salvage pathway III |
| PWY490-4 | asparagine biosynthesis III (tRNA-dependent) |
| PWY66-21 | ethanol degradation II |
| PWY66-400 | glycolysis VI (metazoan) |
| PWYG-321 | mycolate biosynthesis |
| PWYQT-4429 | CO2 fixation into oxaloacetate (anapleurotic) |
| PYRIDNUCSYN-PWY | NAD biosynthesis I (from aspartate) |
| PYRIDOXSYN-PWY | pyridoxal 5-phosphate biosynthesis I |
| PYRUVDEHYD-PWY | pyruvate decarboxylation to acetyl CoA |
| PYRUVOX-PWY | pyruvate oxidation pathway |
| REDCITCYC | TCA cycle III (helicobacter) |
| RHAMCAT-PWY | L-rhamnose degradation I |
| RIBOSYN2-PWY | flavin biosynthesis I (bacteria and plants) |
| SALVADEHYPOX-PWY | adenosine nucleotides degradation II |
| SALVPURINE2-PWY | xanthine and xanthosine salvage |
| SAM-PWY | S-adenosyl-L-methionine biosynthesis |
| SERDEG-PWY | L-serine degradation |
| SERSYN-PWY | serine biosynthesis |
| SORBDEG-PWY | D-sorbitol degradation II |
| SUCROSEUTIL2-PWY | sucrose degradation VII (sucrose 3- |
| | dehydrogenase) |
| SUCUTIL-PWY | sucrose degradation I (sucrose phosphotransferase) |
| TEICHOICACID-PWY | teichoic acid (poly-glycerol) biosynthesis |
| THIOREDOX-PWY | thioredoxin pathway |
| THREONINE-DEG2-PWY | threonine degradation II |
| TREDEGLOW-PWY | trehalose degradation I (low osmolarity) |
| TRESYN-PWY | trehalose biosynthesis I |
| TRNA-CHARGING-PWY | tRNA charging |
| TRPSYN-PWY | tryptophan biosynthesis |
| TRYPDEG-PWY | tryptophan degradation II (via pyruvate) |
| TYRFUMCAT-PWY | tyrosine degradation I |
| TYRSYN | tyrosine biosynthesis I |
| UDPNAGSYN-PWY | UDP-N-acetyl-D-glucosamine biosynthesis I |
| VALDEG-PWY | valine degradation I |
| VALSYN-PWY | valine biosynthesis |
| XYLCAT-PWY | xylose degradation I |

In some embodiments, in addition to core metabolic pathways, high-complexity defined gut microbial communities can further comprise one or more microbes utilizing one or more variable metabolic pathways. As used herein, “variable metabolic pathways” refers to metabolic pathways that are found in some gut metagenomes annotated in the GutCyc project. For example, in some embodiments variable metabolic pathways can include any one or more of the 2AMINOBENZDEG-PWY, 2PHENDEG-PWY, 3-HYDROXYPHENYLACETATE-DEGRADATION-PWY, 7ALPHADEHYDROX-PWY, AEROBACTINSYN-PWY, ALADEG-PWY, ALKANEMONOX-PWY, AMMOXID-PWY, ANAPHENOXI-PWY, ARG-GLU-PWY, ARGDEG-IV-PWY, ARGSPECAT-PWY, ASPARTATE-DEG1-PWY, ASPARTATESYN-PWY, BETSYN-PWY, CALVIN-PWY, CARNMET-PWY, CHOLINE-BETAINE-ANA-PWY, CO2FORM-PWY, CODH-PWY, CYANCAT-PWY, DARABCAT-PWY, DARABITOLUTIL-PWY, DHGLUCONATE-PYR-CAT-PWY, DISSULFRED-PWY, ECASYN-PWY, ENTNER-DOUDOROFF-PWY, FAO-PWY, FESULFOX-PWY, FORMASS-PWY, GALDEG-PWY, GDPRHAMSYN-PWY, GLUCUROCAT-PWY, GLUT-REDOX-PWY, GLUTAMATE-SYN2-PWY, GLYCOLATEMET-PWY, GLYOXDEG-PWY, GLYOXYLATE-BYPASS, GLYSYN-ALA-PWY, HCAMHPDEG-PWY, HOMOCYSDEGR-PWY, HSERMETANA-PWY, IDNCAT-PWY, KDOSYN-PWY, LEU-DEG2-PWY, LIPA-CORESYN-PWY, METHANOGENESIS-PWY, NADPHOS-DEPHOS-PWY, OCTOPINEDEG-PWY, ORN-AMINOPENTANOATE-CAT-PWY, P1-PWY, P101-PWY, P108-PWY, P141-PWY, P161-PWY, P181-PWY, P183-PWY, P184-PWY, P201-PWY, P224-PWY, P241-PWY, P261-PWY, P303-PWY, P341-PWY, P344-PWY, P483-PWY, P541-PWY, P561-PWY, P621-PWY, P641-PWY, PARATHION-DEGRADATION-PWY, PCEDEG-PWY, PROTOCATECHUATE-ORTHO-CLEAVAGE-PWY, PUTDEG-PWY, PWY-101, PWY-1263, PWY-1281, PWY-1341, PWY-1361, PWY-1641, PWY-1723, PWY-1781, PWY-1801, PWY-181, PWY-1861, PWY-2, PWY-2201, PWY-2242, PWY-2503, PWY-2721, PWY-2722, PWY-283, PWY-3161, PWY-3181, PWY-3462, PWY-3602, PWY-3661, PWY-3722, PWY-3941, PWY-40, PWY-4181, PWY-4521, PWY-4601, PWY-481, PWY-4821, PWY-4861, PWY-5025, PWY-5026, PWY-5028, PWY-5033, PWY-5055, PWY-5074, PWY-5080, PWY-5087, PWY-5103, PWY-5111, PWY-5120, PWY-5123, PWY-5154, PWY-5159, PWY-5169, PWY-5177, PWY-5189, PWY-5197, PWY-5198, PWY-5209, PWY-5247, PWY-5250, PWY-5254, PWY-5274, PWY-5276, PWY-5277, PWY-5279, PWY-5280, PWY-5302, PWY-5331, PWY-5332, PWY-5352, PWY-5358, PWY-5364, PWY-5372, PWY-5392, PWY-5437, PWY-5453, PWY-5482, PWY-5484, PWY-5486, PWY-5489, PWY-5490, PWY-5499, PWY-5517, PWY-5519, PWY-5521, PWY-5526, PWY-5530, PWY-5531, PWY-5532, PWY-5533, PWY-5534, PWY-5535, PWY-5651, PWY-5654, PWY-5656, PWY-5662, PWY-5663, PWY-5670, PWY-5675, PWY-5687, PWY-5691, PWY-5697, PWY-5726, PWY-5731, PWY-5739, PWY-5740, PWY-5744, PWY-5747, PWY-5755, PWY-5766, PWY-5782, PWY-5796, PWY-5805, PWY-5810, PWY-5834, PWY-5907, PWY-5915, PWY-5917, PWY-5927, PWY-5929, PWY-5938, PWY-5939, PWY-5951, PWY-5963, PWY-5966, PWY-5979, PWY-5981, PWY-5983, PWY-5985, PWY-5986, PWY-6004, PWY-6021, PWY-6048, PWY-6050, PWY-6060, PWY-6077, PWY-6082, PWY-6107, PWY-6120, PWY-6123, PWY-6125, PWY-6126, PWY-6130, PWY-6137, PWY-6148, PWY-6160, PWY-6166, PWY-6167, PWY-6174, PWY-6183, PWY-6184, PWY-6190, PWY-6213, PWY-6223, PWY-6262, PWY-6281, PWY-6328, PWY-6373, PWY-6383, PWY-6388, PWY-6390, PWY-6406, PWY-6409, PWY-6416, PWY-6419, PWY-6424, PWY-6454, PWY-6455, PWY-6458, PWY-6463, PWY-6464, PWY-6466, PWY-6478, PWY-6481, PWY-6482, PWY-6497, PWY-6499, PWY-6502, PWY-6510, PWY-6523, PWY-6535, PWY-6536, PWY-6537, PWY-6550, PWY-6556, PWY-6580, PWY-6588, PWY-6605, PWY-6611, PWY-6618, PWY-6619, PWY-6622, PWY-6626, PWY-6637, PWY-6644, PWY-6646, PWY-6654, PWY-6655, PWY-6672, PWY-6675, PWY-6679, PWY-6682, PWY-6687, PWY-6690, PWY-6696, PWY-6698, PWY-6711, PWY-6713, PWY-6717, PWY-6728, PWY-6731, PWY-6748, PWY-6753, PWY-6754, PWY-6755, PWY-6759, PWY-6767, PWY-6771, PWY-6789, PWY-6790, PWY-6793, PWY-6795, PWY-6797, PWY-6805, PWY-6813, PWY-6814, PWY-6821, PWY-6825, PWY-6891, PWY-6945, PWY-6966, PWY-6972, PWY-6978, PWY-6993, PWY-6994, PWY-7013, PWY-7014, PWY-7022, PWY-7032, PWY-7046, PWY-7052, PWY-7072, PWY-7097, PWY-7098, PWY-7104, PWY-7106, PWY-7130, PWY-7177, PWY-7198, PWY-7206, PWY-722, PWY-7241, PWY-7254, PWY-7255, PWY-7301, PWY-7309, PWY-7312, PWY-7315, PWY-7316, PWY-7318, PWY-761, PWY-822, PWY-922, PWY0-1221, PWY0-1297, PWY0-1298, PWY0-1321, PWY0-1329, PWY0-1335, PWY0-1336, PWY0-1337, PWY0-1347, PWY0-1348, PWY0-1352, PWY0-1355, PWY0-1356, PWY0-1391, PWY0-1433, PWY0-1465, PWY0-1466, PWY0-1471, PWY0-1533, PWY0-1544, PWY0-163, PWY0-166, PWY0-181, PWY0-301, PWY0-44, PWY0-521, PWY0-981, PWY1G-0, PWY1G-170, PWY3DJ-11281, PWY3O-246, PWY3O-450, PWY490-3, PWY5F9-12, PWYQT-4427, PYRIDNUCSAL-PWY, QUINATEDEG-PWY, RIBITOLUTIL-PWY, RIBOKIN-PWY, RUMP-PWY, SHIKIMATEDEG-PWY, SUCSYN-PWY, TAURINEDEG-PWY, TCA, THRDLCTCAT-PWY, TOLUENE-DEG-2-OH-PWY, TOLUENE-DEG-3-OH-PWY, TOLUENE-DEG-4-OH-PWY, TRPCAT-PWY, and TRPKYNCAT-PWY MetaCyc pathways.

The common names of the variable MetaCyc pathways described above are provided in Table 10 below.

TABLE 10
Variable MetaCyc Pathways

| MetaCyc Pathway ID | Pathway Common Name |
|---|---|
| 2AMINOBENZDEG- | anthranilate degradation III (anaerobic) |
| PWY | |
| 2PHENDEG-PWY | phenylethylamine degradation I |
| 3-HYDROXY- | 4-hydroxyphenylacetate degradation |
| PHENYLACETATE- | |
| DEGRADATION-PWY | |
| 7ALPHADE- | cholate degradation (bacteria, anaerobic) |
| HYDROX-PWY | |
| AEROBACTINSYN- | aerobactin biosynthesis |
| PWY | |
| ALADEG-PWY | alanine degradation I |
| ALKANEMONOX- | two-component alkanesulfonate |
| PWY | monooxygenase |
| AMMOXID-PWY | ammonia oxidation 1 (aerobic) |
| ANAPHENOXI-PWY | phenylalanine degradation II (anaerobic) |
| ARG-GLU-PWY | arginine degradation VII (arginase 3 pathway) |
| ARGDEG-IV-PWY | arginine degradation VIII (arginine oxidase |
| | pathway) |
| ARGSPECAT-PWY | spermine biosynthesis |
| ASPARTATE-DEG1- | aspartate degradation I |
| PWY | |
| ASPARTATESYN- | aspartate biosynthesis |
| PWY | |
| BETSYN-PWY | glycine betaine biosynthesis I (Gram- |
| | negative bacteria) |
| CALVIN-PWY | Calvin-Benson-Bassham cycle |
| CARNMET-PWY | carnitine degradation I |
| CHOLINE-BETAINE- | choline degradation I |
| ANA-PWY | |
| CO2FORM-PWY | methanogenesis from methanol |
| CODH-PWY | reductive acetyl coenzyme A pathway |
| CYANCAT-PWY | cyanate degradation |
| DARABCAT-PWY | D-arabinose degradation II |
| DARABITOLUTIL- | D-arabitol degradation |
| PWY | |
| DHGLUCONATE- | glucose degradation (oxidative) |
| PYR-CAT-PWY | |
| DISSULFRED-PWY | sulfate reduction IV (dissimilatory) |
| ECASYN-PWY | enterobacterial common antigen biosynthesis |
| ENTNER-DOUDOR- | Entner-Doudoroff pathway I |
| OFF-PWY | |
| FAO-PWY | fatty acid beta-oxidation I |
| FESULFOX-PWY | sulfur oxidation II (Fe +3-dependent) |
| FORMASS-PWY | formaldehyde oxidation IV (thiol-independent) |
| GALDEG-PWY | galactose degradation II |
| GDPRHAMSYN-PWY | GDP-D-rhamnose biosynthesis |
| GLUCUROCAT-PWY | superpathway of _-D-glucuronosides |
| | degradation |
| GLUT-REDOX-PWY | glutathione redox reactions II |
| GLUTAMATE- | glutamate biosynthesis II |
| SYN2-PWY | |
| GLYCOLATEMET- | glycolate and glyoxylate degradation I |
| PWY | |
| GLYOXDEG-PWY | glycolate and glyoxylate degradation II |
| GLYOXYLATE- | glyoxylate cycle |
| BYPASS | |
| GLYSYN-ALA-PWY | glycine biosynthesis III |
| HCAMHPDEG-PWY | 3-phenylpropanoate and 3-(3- |
| | hydroxyphenyl)propanoate degradation to 2- |
| | oxopent-4-enoate |
| HOMOCYSDEGR- | cysteine biosynthesis/homocysteine |
| PWY | degradation |
| HSERMETANA-PWY | methionine biosynthesis III |
| IDNCAT-PWY | L-idonate degradation |
| KDOSYN-PWY | KDO transfer to lipid IVA I |
| LEU-DEG2-PWY | leucine degradation I |
| LIPA-CORESYN-PWY | Lipid A-core biosynthesis |
| METHANO- | methanogenesis from CO2 |
| GENESIS-PWY | |
| NADPHOS- | NAD phosphorylation and dephosphorylation |
| DEPHOS-PWY | |
| OCTOPINEDEG-PWY | octopine degradation |
| ORN-AMINOPENTA- | ornithine degradation I (proline biosynthesis) |
| NOATE-CAT-PWY | |
| P1-PWY | purine and pyrimidine metabolism |
| P101-PWY | ectoine biosynthesis |
| P108-PWY | pyruvate fermentation to propionate I |
| P141-PWY | atrazine degradation I (aerobic) |
| P161-PWY | acetylene degradation |
| P181-PWY | nicotine degradation I |
| P183-PWY | catechol degradation to 2-oxopent-4-enoate I |
| P184-PWY | protocatechuate degradation I (meta-cleavage |
| | pathway) |
| P201-PWY | nitroglycerin degradation |
| P224-PWY | sulfate reduction V (dissimilatory) |
| P241-PWY | coenzyme B biosynthesis |
| P261-PWY | coenzyme M biosynthesis I |
| P303-PWY | ammonia oxidation II (anaerobic) |
| P341-PWY | glycolysis V (Pyrococcus) |
| P344-PWY | acrylonitrile degradation |
| P483-PWY | phosphonoacetate degradation |
| P541-PWY | glycine betaine biosynthesis IV (from glycine) |
| P561-PWY | stachydrine degradation |
| P621-PWY | nylon-6 oligomer degradation |
| P641-PWY | phenylmercury acetate degradation |
| PARATHION- | parathion degradation |
| DEGRADATION-PWY | |
| PCEDEG-PWY | tetrachloroethene degradation |
| PROTOCATECHUATE- | protocatechuate degradation II (ortho- |
| ORTHO-CLEAVAGE- | cleavage pathway) |
| PWY | |
| PUTDEG-PWY | putrescine degradation I |
| PWY-101 | photosynthesis light reactions |
| PWY-1263 | taurine degradation I |
| PWY-1281 | sulfoacetaldehyde degradation I |
| PWY-1341 | phenylacetate degradation II (anaerobic) |
| PWY-1361 | benzoyl-CoA degradation I (aerobic) |
| PWY-1641 | methane oxidation to methanol I |
| PWY-1723 | formaldehyde oxidation VI (H4MPT pathway) |
| PWY-1781 | beta-alanine degradation II |
| PWY-1801 | formaldehyde oxidation II (glutathione- |
| | dependent) |
| PWY-181 | photorespiration |
| PWY-1861 | formaldehyde assimilation II (RuMP Cycle) |
| PWY-2 | putrescine degradation IV |
| PWY-2201 | folate transformations I |
| PWY-2242 | ammonia oxidation III |
| PWY-2503 | benzoate degradation I (aerobic) |
| PWY-2721 | trehalose degradation III |
| PWY-2722 | trehalose degradation IV |
| PWY-283 | benzoate degradation II (aerobic and |
| | anaerobic) |
| PWY-3161 | indole-3-acetate biosynthesis III (bacteria) |
| PWY-3181 | tryptophan degradation VI (via tryptamine) |
| PWY-3462 | phenylalanine biosynthesis II |
| PWY-3602 | carnitine degradation II |
| PWY-3661 | glycine betaine degradation |
| PWY-3722 | glycine betaine biosynthesis II (Gram-positive |
| | bacteria) |
| PWY-3941 | beta-alanine biosynthesis II |
| PWY-40 | putrescine biosynthesis I |
| PWY-4181 | glutathione amide metabolism |
| PWY-4521 | arsenite oxidation (respiratory) |
| PWY-4601 | arsenate reduction (respiratory) |
| PWY-481 | ethylbenzene degradation (anaerobic) |
| PWY-4821 | UDP-D-xylose and UDP-D-glucuronate |
| | biosynthesis |
| PWY-4861 | UDP-D-galacturonate biosynthesis I |
| | (from UDP-D-glucuronate) |
| PWY-5025 | indole-3-acetate biosynthesis IV (bacteria) |
| PWY-5026 | indole-3-acetate biosynthesis V |
| | (bacteria and fungi) |
| PWY-5028 | histidine degradation II |
| PWY-5033 | nicotinate degradation II |
| PWY-5055 | nicotinate degradation III |
| PWY-5074 | mevalonate degradation |
| PWY-5080 | very long chain fatty acid biosynthesis |
| PWY-5087 | glutamate degradation VI (to pyruvate) |
| PWY-5103 | isoleucine biosynthesis III |
| PWY-5111 | CMP-KDO biosynthesis II (from D- |
| | arabinose 5-phosphate) |
| PWY-5120 | geranylgeranyl diphosphate biosynthesis |
| PWY-5123 | trans, trans-farnesyl diphosphate |
| | biosynthesis |
| PWY-5154 | arginine biosynthesis III |
| PWY-5159 | 4-hydroxyproline degradation II |
| PWY-5169 | cyanurate degradation |
| PWY-5177 | glutaryl-CoA degradation |
| PWY-5189 | tetrapyrrole biosynthesis II (from glycine) |
| PWY-5197 | lactate biosynthesis (archaea) |
| PWY-5198 | factor 420 biosynthesis |
| PWY-5209 | methyl-coenzyme M oxidation to CO2 |
| PWY-5247 | methanogenesis from methylamine |
| PWY-5250 | methanogenesis from trimethylamine |
| PWY-5254 | methanofuran biosynthesis |
| PWY-5274 | sulfide oxidation II (sulfide dehydrogenase) |
| PWY-5276 | sulfite oxidation I (sulfite oxidoreductase) |
| PWY-5277 | thiosulfate disproportionation I (thiol- |
| | dependent) |
| PWY-5279 | sulfite oxidation II |
| PWY-5280 | lysine degradation IV |
| PWY-5302 | sulfur disproportionation II (aerobic) |
| PWY-5331 | taurine biosynthesis |
| PWY-5332 | sulfur reduction I |
| PWY-5352 | thiosulfate disproportionation II (non thiol- |
| | dependent) |
| PWY-5358 | tetrathionate reduction I (to thiosulfate) |
| PWY-5364 | sulfur reduction II (via polysulfide) |
| PWY-5372 | carbon tetrachloride degradation II |
| PWY-5392 | reductive TCA cycle II |
| PWY-5437 | threonine degradation I |
| PWY-5453 | methylglyoxal degradation III |
| PWY-5482 | pyruvate fermentation to acetate II |
| PWY-5484 | glycolysis II (from fructose-6P) |
| PWY-5486 | pyruvate fermentation to ethanol II |
| PWY-5489 | methyl parathion degradation |
| PWY-5490 | paraoxon degradation |
| PWY-5499 | vitamin B6 degradation |
| PWY-5517 | L-arabinose degradation III |
| PWY-5519 | D-arabinose degradation III |
| PWY-5521 | L-ascorbate biosynthesis III |
| PWY-5526 | bacteriochlorophyll a biosynthesis |
| PWY-5530 | sorbitol biosynthesis II |
| PWY-5531 | chlorophyllide a biosynthesis II (anaerobic) |
| PWY-5532 | adenosine nucleotides degradation IV |
| PWY-5533 | acetone degradation II (to acetoacetate) |
| PWY-5534 | propylene degradation |
| PWY-5535 | acetate formation from acetyl-CoA II |
| PWY-5651 | tryptophan degradation to 2-amino-3- |
| | carboxymuconate semialdehyde |
| PWY-5654 | 2-amino-3-carboxymuconate semialdehyde |
| | degradation to 2-oxopentenoate |
| PWY-5656 | mannosylglycerate biosynthesis I |
| PWY-5662 | glucosylglycerate biosynthesis I |
| PWY-5663 | tetrahydrobiopterin biosynthesis I |
| PWY-5670 | epoxysqualene biosynthesis |
| PWY-5675 | nitrate reduction V (assimilatory) |
| PWY-5687 | pyrimidine ribonucleotides interconversion |
| PWY-5691 | urate degradation to allantoin |
| PWY-5697 | allantoin degradation to ureidoglycolate |
| | I (urea producing) |
| PWY-5726 | deethyl simazine degradation |
| PWY-5731 | atrazine degradation III |
| PWY-5739 | GDP-D-perosamine biosynthesis |
| PWY-5740 | GDP-L-colitose biosynthesis |
| PWY-5744 | glyoxylate assimilation |
| PWY-5747 | 2-methylcitrate cycle II |
| PWY-5755 | 4-hydroxybenzoate biosynthesis II |
| | (bacteria and fungi) |
| PWY-5766 | glutamate degradation X |
| PWY-5782 | 2-keto-L-gulonate biosynthesis |
| PWY-5796 | 2-(5-phosphoribosyl)-3-dephospho-CoA |
| | biosynthesis II (malonate decarboxylase) |
| PWY-5805 | nonaprenyl diphosphate biosynthesis I |
| PWY-5810 | usnate biosynthesis |
| PWY-5834 | CDP-tyvelose biosynthesis |
| PWY-5907 | homospermidine biosynthesis |
| PWY-5915 | phycoerythrobilin biosynthesis |
| PWY-5917 | phycocyanobilin biosynthesis |
| PWY-5927 | (4S)-carveol and (4S)-dihydrocarveol |
| | degradation |
| PWY-5929 | puromycin biosynthesis |
| PWY-5938 | (R)-acetoin biosynthesis I |
| PWY-5939 | (R)-acetoin biosynthesis II |
| PWY-5951 | (R,R)-butanediol biosynthesis |
| PWY-5963 | thio-molybdenum cofactor biosynthesis |
| PWY-5966 | fatty acid biosynthesis initiation II |
| PWY-5979 | 3-amino-5-hydroxybenzoate biosynthesis |
| PWY-5981 | CDP-diacylglycerol biosynthesis III |
| PWY-5983 | trehalose biosynthesis VI |
| PWY-5985 | trehalose biosynthesis VII |
| PWY-5986 | ammonium transport |
| PWY-6004 | glycine betaine biosynthesis V (from glycine) |
| PWY-6021 | nitrilotriacetate degradation |
| PWY-6048 | methylthiopropionate degradation I (cleavage) |
| PWY-6050 | dimethyl sulfoxide degradation |
| PWY-6060 | malonate degradation II (biotin-dependent) |
| PWY-6077 | anthranilate degradation II (aerobic) |
| PWY-6082 | alginate biosynthesis II |
| PWY-6107 | chlorosalicylate degradation |
| PWY-6120 | tyrosine biosynthesis III |
| PWY-6123 | inosine-5-phosphate biosynthesis I |
| PWY-6125 | guanosine nucleotides de novo biosynthesis |
| PWY-6126 | adenosine nucleotides de novo biosynthesis |
| PWY-6130 | glycerol degradation III |
| PWY-6137 | copper transport II |
| PWY-6148 | tetrahydromethanopterin biosynthesis |
| PWY-6160 | 3-dehydroquinate biosynthesis II (archaea) |
| PWY-6166 | calcium transport I |
| PWY-6167 | flavin biosynthesis II (archaea) |
| PWY-6174 | mevalonate pathway II (archaea) |
| PWY-6183 | salicylate degradation I |
| PWY-6184 | methylsalicylate degradation |
| PWY-6190 | 2,4-dichlorotoluene degradation |
| PWY-6213 | cadmium transport I |
| PWY-6223 | gentisate degradation |
| PWY-6262 | demethylmenaquinol-8 biosynthesis II |
| PWY-6281 | selenocysteine biosynthesis II (archaea and |
| | eukaryotes) |
| PWY-6328 | lysine degradation X |
| PWY-6373 | acrylate degradation |
| PWY-6383 | mono-trans, poly-cis decaprenyl phosphate |
| | biosynthesis |
| PWY-6388 | (S,S)-butanediol degradation |
| PWY-6390 | (S,S)-butanediol biosynthesis |
| PWY-6406 | salicylate biosynthesis I |
| PWY-6409 | pyoverdine I biosynthesis |
| PWY-6416 | quinate degradation II |
| PWY-6419 | shikimate degradation II |
| PWY-6424 | 26,27-dehydrozymosterol metabolism |
| PWY-6454 | vancomycin resistance I |
| PWY-6455 | vancomycin resistance II |
| PWY-6458 | benzoyl-CoA biosynthesis |
| PWY-6463 | peptidoglycan cross-bridge biosynthesis IV |
| | (Weissella viridescens) |
| PWY-6464 | polyvinyl alcohol degradation |
| PWY-6466 | pyridoxal 5-phosphate biosynthesis II |
| PWY-6478 | GDP-D-glycero-alpha-D-manno-heptose |
| | biosynthesis |
| PWY-6481 | L-dopachrome biosynthesis |
| PWY-6482 | diphthamide biosynthesis |
| PWY-6497 | D-galactarate degradation II |
| PWY-6499 | D-glucarate degradation II |
| PWY-6502 | oxidized GTP and dGTP detoxification |
| PWY-6510 | methanol oxidation to formaldehyde II |
| PWY-6523 | intra-aerobic nitrite reduction |
| PWY-6535 | 4-aminobutyrate degradation I |
| PWY-6536 | 4-aminobutyrate degradation III |
| PWY-6537 | 4-aminobutyrate degradation II |
| PWY-6550 | carbazole degradation |
| PWY-6556 | pyrimidine ribonucleosides degradation II |
| PWY-6580 | L-1-phosphatidyl-inositol biosynthesis |
| | (Mycobacteria) |
| PWY-6588 | pyruvate fermentation to acetone |
| PWY-6605 | adenine and adenosine salvage II |
| PWY-6611 | adenine and adenosine salvage V |
| PWY-6618 | guanine and guanosine salvage III |
| PWY-6619 | adenine and adenosine salvage VI |
| PWY-6622 | heptadecane biosynthesis |
| PWY-6626 | CDP-2-glycerol biosynthesis |
| PWY-6637 | sulfolactate degradation II |
| PWY-6644 | fluoroacetate and fluorothreonine |
| | biosynthesis |
| PWY-6646 | fluoroacetate degradation |
| PWY-6654 | phosphopantothenate biosynthesis III |
| PWY-6655 | xanthan biosynthesis |
| PWY-6672 | cis-genanyl-CoA degradation |
| PWY-6675 | sulfur oxidation IV (intracellular sulfur) |
| PWY-6679 | jadomycin biosynthesis |
| PWY-6682 | dehydrophos biosynthesis |
| PWY-6687 | mannosylglucosylglycerate biosynthesis II |
| PWY-6690 | cinnamate and 3-hydroxycinnamate |
| | degradation to 2-oxopent-4-enoate |
| PWY-6696 | oxalate degradation III |
| PWY-6698 | oxalate degradation V |
| PWY-6711 | archaeosine biosynthesis |
| PWY-6713 | L-rhamnose degradation II |
| PWY-6717 | (1,4)-beta-xylan degradation |
| PWY-6728 | methylaspartate cycle |
| PWY-6731 | starch degradation III |
| PWY-6748 | nitrate reduction VII (denitrification) |
| PWY-6753 | S-methyl-5-thioadenosine degradation III |
| PWY-6754 | S-methyl-5-thioadenosine degradation I |
| PWY-6755 | S-methyl-5-thio-alpha-D-ribose 1-phosphate |
| | degradation I |
| PWY-6759 | hydrogen production III |
| PWY-6767 | 4,4-diapolycopenedioate biosynthesis |
| PWY-6771 | rhamnogalacturonan type I degradation |
| | II (bacteria) |
| PWY-6789 | (l,3)-beta-D-xylan degradation |
| PWY-6790 | L-arabinan degradation |
| PWY-6793 | demethylmenaquinol-8 biosynthesis III |
| PWY-6795 | diacylglyceryl-N,N,N-trimethylhomoserine |
| | biosynthesis |
| PWY-6797 | 6-hydroxymethyl-dihydropterin diphosphate |
| | biosynthesis II (archaea) |
| PWY-6805 | cellulose degradation I (cellulosome) |
| PWY-6813 | glucuronoarabinoxylan degradation |
| PWY-6814 | acidification and chitin degradation |
| | (in carnivorous plants) |
| PWY-6821 | kappa-carrageenan degradation |
| PWY-6825 | phosphatidylcholine biosynthesis V |
| PWY-6891 | thiazole biosynthesis II (Bacillus) |
| PWY-6945 | cholesterol degradation to androstenedione |
| | I (cholesterol oxidase) |
| PWY-6966 | methanol oxidation to formaldehyde I |
| PWY-6972 | oleandomycin activation/inactivation |
| PWY-6978 | plastoquinol-9 biosynthesis II |
| PWY-6993 | nicotine degradation II |
| PWY-6994 | pyrrolysine biosynthesis |
| PWY-7013 | L-l,2-propanediol degradation |
| PWY-7014 | paromamine biosynthesis I |
| PWY-7022 | paromamine biosynthesis II |
| PWY-7032 | alkane biosynthesis I |
| PWY-7046 | 4-coumarate degradation (anaerobic) |
| PWY-7052 | cyanophycin metabolism |
| PWY-7072 | hopanoid biosynthesis (bacteria) |
| PWY-7097 | vanillin and vanillate degradation I |
| PWY-7098 | vanillin and vanillate degradation II |
| PWY-7104 | dTDP-L-megosamine biosynthesis |
| PWY-7106 | erythromycin D biosynthesis |
| PWY-7130 | L-glucose degradation |
| PWY-7177 | UTP and CTP dephosphorylation II |
| PWY-7198 | pyrimidine deoxyribonucleotides de novo |
| | biosynthesis IV |
| PWY-7206 | pyrimidine deoxyribonucleotides |
| | dephosphorylation |
| PWY-722 | nicotinate degradation I |
| PWY-7241 | myo-inositol degradation II |
| PWY-7254 | TCA cycle VII (acetate-producers) |
| PWY-7255 | ergothioneine biosynthesis |
| PWY-7301 | dTDP-beta-L-noviose biosynthesis |
| PWY-7309 | acrylonitrile degradation II |
| PWY-7312 | dTDP-D-beta-fucofuranose biosynthesis |
| PWY-7315 | dTDP-N-acetylthomosamine biosynthesis |
| PWY-7316 | dTDP-N-acetylviosamine biosynthesis |
| PWY-7318 | dTDP-3-acetamido-3,6-dideoxy-alpha- |
| | D-glucose biosynthesis |
| PWY-761 | rhizobactin 1021 biosynthesis |
| PWY-822 | fructan biosynthesis |
| PWY-922 | mevalonate pathway I |
| PWY0-1221 | putrescine degradation II |
| PWY0-1297 | superpathway of purine |
| | deoxyribonucleosides degradation |
| PWY0-1298 | superpathway of pyrimidine deoxy- |
| | ribonucleosides degradation |
| PWY0-1321 | nitrate reduction III (dissimilatory) |
| PWY0-1329 | succinate to cytochrome bo oxidase |
| | electron transfer |
| PWY0-1335 | NADH to cytochrome bo oxidase |
| | electron transfer |
| PWY0-1336 | NADH to fumarate electron transfer |
| PWY0-1337 | oleate beta-oxidation |
| PWY0-1347 | NADH to trimethylamine N-oxide |
| | electron transfer |
| PWY0-1348 | NADH to dimethyl sulfoxide |
| | electron transfer |
| PWY0-1352 | nitrate reduction VIII (dissimilatory) |
| PWY0-1355 | formate to trimethylamine N-oxide |
| | electron transfer |
| PWY0-1356 | formate to dimethyl sulfoxide |
| | electron transfer |
| PWY0-1391 | S-methyl-5-thioadenosine degradation IV |
| PWY0-1433 | tetrahydromonapterin biosynthesis |
| PWY0-1465 | D-malate degradation |
| PWY0-1466 | trehalose degradation VI (periplasmic) |
| PWY0-1471 | uracil degradation III |
| PWY0-1533 | methylphosphonate degradation |
| PWY0-1544 | proline to cytochrome bo oxidase |
| | electron transfer |
| PWY0-163 | salvage pathways of pyrimidine |
| | ribonucleotides |
| PWY0-166 | superpathway of pyrimidine deoxy- |
| | ribonucleotides de novo biosynthesis |
| | (E. coli) |
| PWY0-181 | salvage pathways of pyrimidine |
| | deoxy rib onucl eoti des |
| PWY0-301 | L-ascorbate degradation I (bacterial, |
| | anaerobic) |
| PWY0-44 | D-allose degradation |
| PWY0-521 | fructoselysine and psicoselysine |
| | degradation |
| PWY0-981 | taurine degradation IV |
| PWY1G-0 | mycothiol biosynthesis |
| PWY1G-170 | formaldehyde oxidation III (mycothiol- |
| | dependent) |
| PWY3DJ-11281 | sphingomyelin metabolism |
| PWY3O-246 | (R,R)-butanediol degradation |
| PWY3O-450 | phosphatidylcholine biosynthesis I |
| PWY490-3 | nitrate reduction VI (assimilatory) |
| PWY5F9-12 | biphenyl degradation |
| PWYQT-4427 | sulfolipid biosynthesis |
| PYRJDNUCSAL-PWY | NAD salvage pathway I |
| QUINATEDEG-PWY | quinate degradation I |
| RIBITOLUTIL-PWY | ribitol degradation |
| RIBOKIN-PWY | ribose degradation |
| RUMP-PWY | formaldehyde oxidation I |
| SHIKIMATEDEG-PWY | shikimate degradation I |
| SUCSYN-PWY | sucrose biosynthesis |
| TAURINEDEG-PWY | taurine degradation III |
| TCA | TCA cycle I (prokaryotic) |
| THRDLCTCAT-PWY | threonine degradation III (to methylglyoxal) |
| TOLUENE-DEG- | toluene degradation to 2-oxopent-4-enoate I |
| 2-OH-PWY | (via o-cresol) |
| TOLUENE-DEG- | toluene degradation to 2-oxopent-4-enoate |
| 3-OH-PWY | (via 4-methylcatechol) |
| TOLUENE-DEG- | toluene degradation to protocatechuate |
| 4-OH-PWY | (via p-cresol) |
| TRPCAT-PWY | tryptophan degradation I (via anthranilate) |
| TRPKYNCAT-PWY | tryptophan degradation IV (via indole- |
| | 3-lactate) |

In some embodiments, the comprehensive panel of substrates or nutrients metabolized by a metabolic pathway include a-mannan (yeast), acetate, agarose, alanine, alginate, anthocyanin, arabinan, arabinogalactan, arabinoxylan, arginine, asparagine, Aspartate, b-glucans, butyrate, carrageenan, chitin, chlorogenic acids, chondroitin sulfate, cinnamic acid, Cysteine, dextran (40), Dihydrochalcones, Enterodiol, flavan-3-ols, flavanones, flavones, flavonols, folate, formate, galactomannan (carob), galacturonan (homo), galacturonate, glucomannan (konjac), glutamate, Glutamine, Glycine, Histidine, hyaluronan, hydrogen, hydroxycinnamic acids, hydroxyproline, inulin, isoflavones/isoflavanones, Isoleucine, lactate, laminarin, Leucine, levan, Lysine, Methionine, mucin O-linked glycans, Ornithine, Phenylalanine, porphyran, Proline, propionate, rhamnogalacturonan I, rhamnogalacturonan II, Secoisolariciresinol diglucoside, Serine, starch (potato), starch (structure 1), thiamine, Threonine, tryptophan, Tyrosine, Valine, xyloglucan, and xylooligosaccharides (XOS). For example, in some embodiments, the comprehensive panel of substrates or nutrients metabolized by a metabolic pathway comprises at least 90%, at least 91%, at least 92%, at least 93%, at least 94%, at least 95%, at least 96%, at least 97%, at least 98%, at least 99%, or all the substrates described above.

In some embodiments, the comprehensive panel of metabolites synthesized or produced by a metabolic pathway include formate, acetate, propionate, butyrate, isobutyrate, valerate, isovalerate, 2-methylbutyrate, caporate, isocaporate, 3-methylvaleric acid, L-phenylalanine, 3-phenylpropionic acid, phyenypyruvate, DL-3-phenyllactic acid, trans-cinnamic acid, phenyllactic acid, phenethylamine, L-tyrosine, 3-(4-hydroxyphenyl)propionic acid, 3-(4-hydroxyphenyl) pyruvic acid, DL-p-hydroxyphenyl lactic acid, p-coumaric acid, 4-hydroxyphenyl acetic acid, tyramine, phenol, p-cresol, 4-ethylphenol, 4-vinylphenol, 4-hydroxybenzoic acid, L-tryptophan, 3-indolepropionic acid, 3-indolepyruvic acid, DL-indole-3-lactic acid, trans-3-indoleacrylic acid, 3-indoleacetic acid, tryptamine, indole, skatol, indole-3-carboxylic acid, indole-3-carboxyaldehyde, N-acetyl-L-phenylalanine, phenylpropionylglycine, 3-(3-hydroxyphenyl) propionic acid, cinnamoylglycine, phenylacetylglycine, phenylacetylglutamine, hippuric acid, 2-hydroxyhippuric acid, 3-hydroxyhippuric acid, 4-hydroxyhippuric acid, 4-hydroxyphenylacetylglycine, phenyl sulfate, phenyl glucuronide, p-cresol sulfate, p-cresol glucuronide, 4-ethylphenol sulfate, 4-ethylphenol glucuronide, N-acetyl-L-tryptophan, 5-hydroxy-L-typtophan, N-acetyl serotonin, 3-indolepriopionylglycine, indolyl-3-acryloylglycine, indoxyl sulfate, indoxyl glucuronide, 5-hydroxyindole-3-acetic acid, indoleacetylglycine, lithocholic acid, murocholic acid, ursodeoxycholic acid, hyodeoxycholic acid, deoxycholic acid, 7-oxocholic acid, ω-muricholic acid, α-muricholic acid, β-muricholic acid, γ-muricholic acid, 7f3cholic acid, taurolithocholic acid, tauroursodeoxycholic acid, taurohyodeoxycholic acid, taurochenodeoxycholic acid, taurodeoxycholic acid, tauro-β-muricholic acid, tauro-ω-muricholic acid, and taurocholic acid. For example, in some embodiments, the comprehensive panel of metabolites synthesized or produced by a metabolic pathway comprises at least 90%, at least 91%, at least 92%, at least 93%, at least 94%, at least 95%, at least 96%, at least 97%, at least 98%, at least 99%, or all the substrates described above.

In some embodiments, high-complexity defined gut microbial communities disclosed herein are assembled to have the ability to metabolize at least 90% (e.g., at least 91%, at least 92%, at least 93%, at least 94%, at least 95%, at least 96%, at least 97%, at least 98%, at least 99%, or all) of the substrates described above and produce at least 90% (e.g., at least 91%, at least 92%, at least 93%, at least 94%, at least 95%, at least 96%, at least 97%, at least 98%, at least 99%, or all) of the metabolites described above.

In some embodiments, high-complexity defined gut microbial communities disclosed herein are assembled to have the ability to metabolize at least 90% (e.g., at least 91%, at least 92%, at least 93%, at least 94%, at least 95%, at least 96%, at least 97%, at least 98%, at least 99%, or all) of the substrates described above and utilize at least 90% (e.g., at least 91%, at least 92%, at least 93%, at least 94%, at least 95%, at least 96%, at least 97%, at least 98%, at least 99%, or all) of the MetaCyc metabolic pathways described above.

In some embodiments, high-complexity defined gut microbial communities disclosed herein are assembled to have the ability to produce at least 90% (e.g., at least 91%, at least 92%, at least 93%, at least 94%, at least 95%, at least 96%, at least 97%, at least 98%, at least 99%, or all) of the metabolites described above and utilize at least 90% (e.g., at least 91%, at least 92%, at least 93%, at least 94%, at least 95%, at least 96%, at least 97%, at least 98%, at least 99%, or all) of the MetaCyc metabolic pathways described above.

In some embodiments, high-complexity defined gut microbial communities disclosed herein are assembled to have the ability to metabolize at least 90% (e.g., at least 91%, at least 92%, at least 93%, at least 94%, at least 95%, at least 96%, at least 97%, at least 98%, at least 99%, or all) of the substrates described above, produce at least 90% (e.g., at least 91%, at least 92%, at least 93%, at least 94%, at least 95%, at least 96%, at least 97%, at least 98%, at least 99%, or all) of the metabolites described above, and utilize at least 90% (e.g., at least 91%, at least 92%, at least 93%, at least 94%, at least 95%, at least 96%, at least 97%, at least 98%, at least 99%, or all) of the MetaCyc metabolic pathways described above.

In some embodiments, the ability to metabolize a substrate, produce a metabolite, or utilize a MetaCyc pathway is experimentally determined by culturing the defined gut microbial community in vitro and measuring whether a substrate is metabolized, a metabolite is produced, and/or a reaction intermediate in a MetaCyc pathway is produced by liquid chromatography-mass spectrometry analysis.

In some embodiments, the ability to metabolize a substrate, produce a metabolite, or utilize a MetaCyc pathway is experimentally determined by administering the defined gut microbial community to a gnotobiotic mouse and measuring whether a substrate is metabolized, a metabolite is produced, and/or a reaction intermediate in a MetaCyc pathway is produced after a defined period of time by liquid chromatography-mass spectrometry (LC-MS) analysis of a sample obtained from the mouse. In some embodiments, the defined gut microbial community is administered via a route selected from the group consisting of oral, rectal, fecal (by enema), and naso/oro-gastric gavage. In some embodiments, the defined period of time is about 1 hour, 2 hours, 4 hours, 8 hours, 12 hours, 24 hours, 2 days, 7 days, 14 days, 1 month, or 2 months. In some embodiments, the same obtained from the mouse is selected from the group consisting of a fecal sample, urine sample, blood sample, or serum sample.

### 8. Microbial Communities for the Treatment of Dysbiosis or a Pathological Condition

Backfill communities identified using the methods described herein can be used to treat patients by administration of a high-complexity defined gut microbial community. Exemplary patients are patients with dysbiosis or a pathological condition.

8.1 Clostridium difficile Infection

### 8.1.1 Murine Model

In some embodiments, when tested in a murine model of C. difficile infection, the high-complexity defined microbial community of the present invention reduces the number of C. difficile colony forming units (CFU) per μl of stool by at least 1 to 2 logs, at least 2 to 3 logs, at least 3 to 4 logs, at least 4 to 5 logs, or by at least 5 to 6 logs. In some embodiments, when tested in a murine model of C. difficile infection, the high-complexity defined microbial community of the present invention reduces the number of C. difficile colony forming units (CFU) per gram of stool by at least 1 to 2 logs, at least 2 to 3 logs, at least 3 to 4 logs, at least 4 to 5 logs, or by at least 5 to 6 logs.

8.1.2 Treatment of Persistent C. difficile Infection

In some embodiments, a high-complexity defined gut microbial community of the present invention can be used to treat an animal having a persistent C. difficile infection. For example in some embodiments, the animal may be a mammal, and more particularly a human.

In some embodiments, a method for producing a high-complexity defined gut microbial community of the present invention for treatment of persistent C. difficile infection, may comprise: i) performing a C. difficile plate count on a stool sample obtained from an animal having a persistent C. difficile infection; ii) engrafting the high-complexity defined gut microbial community into the gut of the animal having a persistent C. difficile infection to produce an engrafted, infected animal; iii) maintaining the engrafted, infected animal for a time sufficient for enteric colonization by microbial strains of the high-complexity defined gut microbial community, thereby producing an engrafted, infected community in the gut of the engrafted, infected animal; iv) performing an additional C. difficile plate count on a stool sample obtained from the engrafted, infected animal; v) if the number of C. difficile CFUs obtained from the plate count of step iv) is not significantly less than the number of C. difficile CFUs obtained from the plate count of step i), adding one or more than one additional defined microbial strain to the high-complexity defined gut microbial community that was not present in step ii) to produce a modified, high-complexity defined gut microbial community and repeating steps i) to iv) in an animal having a persistent C. difficile infection that has never been engrafted, using the modified, high-complexity defined gut microbial community as the high-complexity defined gut microbial community; and if there is a statistically significant reduction in the number of C. difficile CFUs obtained from the plate count of step iv) as compared to the number of C. difficile CFUs obtained from the plate count of step i), the modified, defined, stable enteric community in the final step iv) is a final, high-complexity defined gut microbial community.

In some embodiments, administration of an effective amount of final, high-complexity defined gut microbial community to an animal having a persistent C. difficile infection effectively reduces the number of C. difficile CFU/μ1 of stool in the treated animal. In some embodiments, administration of an effective amount of final, high-complexity defined gut microbial community to an animal having a persistent C. difficile infection effectively reduces the number of C. difficile CFU/g of stool in the treated animal.

### 8.2 Bile Acid Metabolism and Cholestatic Disease

In some embodiments, a high-complexity defined gut microbial community significantly alters the profile and/or concentration of bile acids present in an animal (e.g., mouse) stool sample as compared to an isogenic gnotobiotic control animal (e.g., isogenic gnotobiotic control mouse).

For example, in some embodiments, a high-complexity defined gut microbial community of the present invention significantly alters the profile and/or concentration of Tβ-MCA, Tα-MCA, TUDCA, THDCA, TCA, 7β-CA, 7-oxo-CA, TCDCA, Tω-MCA, TDCA, α-MCA, β-MCA, ω-MCA, Muro-CA, d4-CA, CA, TLCA, UDCA, HDCA, CDCA, DCA, and LCA in an animal (e.g. mouse).

In some embodiments, a high-complexity defined gut microbial community of the present invention can be used to treat an animal having a cholestatic disease, such as, for example, primary sclerosing cholangitis, primary biliary cholangitis, progressive familial intrahepatic cholestasis, or nonalcoholic steatohepatitis. For example in some embodiments, the animal may be a mammal, and more particularly a human.

### 9. Modification of Metabolites

In some embodiments, a high-complexity defined gut microbial community significantly alters the concentration of metabolites present in an animal (e.g., mouse) urine sample as compared to an isogenic gnotobiotic control animal (e.g. isogenic gnotobiotic control mouse).

For example in some embodiments, a high-complexity defined gut microbial community of the present invention significantly alters the concentration of 4-hydroxybenzoic acid, L-tyrosine, 4-hydroxyphenylacetic acid, DL-p-hydroxyphenyllactic acid, p-coumaric acid, 3-(4-Hydroxyphenyl) propionic acid, 3-(4-hydroxyphenyl)pyruvic acid, indole-3-carboxylic acid, tyramine, L-phenylalanine, phenylacetic acid, 3-indoleacetic acid, DL-3-phenyllactic acid, L-tryptophan, DL-indole-3-lactic acid, phenylpyruvate, trans-3-indoleacrylic acid, 3-indolepyruvic acid, 3-indolepyropionic acid, 3-phenylproprionic acid, trans-cinnamic acid, tryptamine, phenol, indole-3-carboxaldehyde, p-cresol, indole, 4-vinylphenol, or 4-ethylphenol.

### 10. Pharmaceutical Compositions

A product of the in vivo backfill process is a defined microbial community (e.g., a stable defined microbial community) with a known phenotype (e.g., a metabolic phenotype) that, when engrafted into a subject, confers benefit to the subject.

The therapeutic backfill community may be expanded and combined with excipients for administration orally (e.g., as a capsule), by naso/oro-gastric gavage, fecally (e.g. by enema), or rectally (e.g., by colonoscopy). Exemplary excipients include normal saline and others known in the art.

The present disclosure also provides pharmaceutical compositions that contain an effective amount of a microbial community, e.g., a high-complexity defined gut microbial community. The composition can be formulated for use in a variety of delivery systems. One or more physiologically acceptable excipient(s) or carrier(s) can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

In some embodiments a pharmaceutical composition disclosed herein may comprise a microbial community, e.g., a high-complexity defined gut microbial community, of the present invention and one or more than one agent selected from, but not limited to: carbohydrates (e.g., glucose, sucrose, galactose, mannose, ribose, arabinose, xylose, fructose, maltose, cellobiose, lactose, deoxyribose, hexose); lipids (e.g., lauric acid (12:0) myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0)); minerals (e.g., chloride, sodium, calcium, iron, chromium, copper, iodine, zinc, magnesium, manganese, molybdenum, phosphorus, potassium, and selenium); vitamins (e.g., vitamin C, vitamin A, vitamin E, vitamin B 12, vitamin K, riboflavin, niacin, vitamin D, vitamin B6, folic acid, pyridoxine, thiamine, pantothenic acid, and biotin); buffering agents (e.g., sodium citrate, magnesium carbonate, magnesium bicarbonate, calcium carbonate, and calcium bicarbonate); preservatives (e.g., alpha-tocopherol, ascorbate, parabens, chlorobutanol, and phenol); binders (e.g., starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides); lubricants (e.g., magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil); dispersants (e.g., starch, alginic acid, polyvinylpyrrolidones, guar gum, kaolin, bentonite, purified wood cellulose, sodium starch glycolate, isomorphous silicate, and microcrystalline cellulose); disintegrants (e.g., corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, tragacanth, sodium bicarbonate in combination with citric acid, and sodium bicarbonate in combination with tartaric acid); flavoring agents; sweeteners; and coloring agents.

In certain embodiments, a microbial community, e.g., a high-complexity defined gut microbial community, of the present invention is administered orally as a lyophilized powder, capsule, tablet, troche, lozenge, granule, gel or liquid. In some embodiments, a microbial community, e.g., a high-complexity defined gut microbial community, of the present invention is administered as a tablet or pill and can be compressed, multiply compressed, multiply layered, and/or coated.

### 11. Dosages

In some embodiments, a microbial community, e.g., a high-complexity defined gut microbial community of the present invention is administered in a dosage form having a total amount of microbial community, e.g., a high-complexity defined gut microbial community, of 1×10⁶ to 1×10¹³ CFUs, 1×10⁶ to 1×10¹² CFUs, 1×10⁶ to 1×10¹¹ CFUs, 1×10⁶ to 1×10¹⁰ CFUs, 1×10⁶ to 1×10⁹ CFUs, 1×10⁶ to 1×10⁸ CFUs, 1×10⁶ to 1×10⁷ CFUs, 5×10⁶ to 1×10¹³ CFUs, 5×10⁶ to 1×10¹² CFUs, 5×10⁶ to 1×10¹¹ CFUs, 5×10⁶ to 1×10¹⁰ CFUs, 5×10⁶ to 1×10⁹ CFUs, 5×10⁶ to 1×10⁸ CFUs, 5×10⁶ to 1×10⁷ CFUs, 1×10⁷ to 1×10¹³ CFUs, 1×10⁷ to 1×10¹² CFUs, 1×10⁷ to 1×10¹¹ CFUs, 1×10⁷ to 1×10¹⁰ CFUs, 1×10⁷ to 1×10⁹ CFUs, 1×10⁷ to 1×10⁸ CFUs, 5×10⁷ to 1×10¹³ CFUs, 5×10⁷ to 1×10¹² CFUs, 5×10⁷ to 1×10¹¹ CFUs, 5×10⁷ to 1×10¹⁰ CFUs, 5×10⁷ to 1×10⁹ CFUs, 5×10⁷ to 1×10⁸ CFUs, 1×10⁸ to 1×10¹³ CFUs, 1×10⁸ to 1×10¹² CFUs, 1×10⁸ to 1×10¹¹ CFUs, 1×10⁸ to 1×10¹⁰ CFUs, 1×10⁸ to 1×10⁹ CFUs, 5×10⁸ to 1×10¹³ CFUs, 5×10⁸ to 1×10¹² CFUs, 5×10⁸ to 1×10¹¹ CFUs, 5×10⁸ to 1×10¹⁰ CFUs, 5×10⁸ to 1×10⁹ CFUs, 1×10⁹ to 1×10¹³ CFUs, 1×10⁹ to 1×10¹² CFUs, 1×10⁹ to 1×10¹¹ CFUs, 1×10⁹ to 1×10¹⁰ CFUs, 5×10⁹ to 1×10¹³ CFUs, 5×10⁹ to 1×10¹² CFUs, 5×10⁹ to 1×10¹¹ CFUs, 5×10⁹ to 1×10¹⁰ CFUs, 1×10¹⁰ to 1×10¹³ CFUs, 1×10¹⁰ to 1×10¹² CFUs, 1×10¹⁰ to 1×10¹¹ CFUs, 5×10¹⁰ to 1×10¹³ CFUs, 5×10¹⁰ to 1×10¹² CFUs or 5×10¹⁰ to 1×10¹¹ CFUs.

In some embodiments, a microbial community, e.g., a high-complexity defined gut microbial community of the present invention is administered in a dosage form having a total amount of microbial community, e.g., a high-complexity defined gut microbial community, of 0.1 ng to 500 mg, 0.5 ng to 500 mg, 1 ng to 500 mg, 5 ng to 500 mg, 10 ng to 500 mg, 50 ng to 500 mg, 100 ng to 500 mg, 500 ng to 500 mg, 1 μg to 500 mg, 5 μg to 500 mg, 10 μg to 500 mg, 50 μg to 500 mg, 100 μg to 500 mg, 500 μg to 500 mg, 1 mg to 500 mg, 5 mg to 500 mg, 10 mg to 500 mg, 50 mg to 500 mg, 100 mg to 500 mg, 0.1 ng to 100 mg, 0.5 ng to 100 mg, 1 ng to 100 mg, 5 ng to 100 mg, 10 ng to 100 mg, 50 ng to 100 mg, 100 ng to 100 mg, 500 ng to 500 mg, 1 to 100 mg, 5 μg to 100 mg, 10 μg to 100 mg, 50 μg to 100 mg, 100 μg to 100 mg, 500 μg to 100 mg, 1 mg to 500 mg, 5 mg to 100 mg, 10 mg to 100 mg, 50 mg to 100 mg, 0.1 ng to 50 mg, 0.5 ng to 50 mg, 1 ng to 50 mg, 5 ng to 50 mg, 10 ng to 50 mg, 50 ng to 50 mg, 100 ng to 50 mg, 500 ng to 500 mg, 1 μs to 50 mg, 5 μg to 50 mg, 10 μs to 50 mg, 50 μs to 50 mg, 100 μg to 50 mg, 500 μg to 50 mg, 1 mg to 500 mg, 5 mg to 50 mg, 10 mg to 50 mg, 0.1 ng to 10 mg, 0.5 ng to 10 mg, 1 ng to 10 mg, 5 ng to 10 mg, 10 ng to 10 mg, 50 ng to 10 mg, 100 ng to 10 mg, 500 ng to 500 mg, 1 μg to 10 mg, 5 μg to 10 mg, 10 μg to 10 mg, 50 μg to 10 mg, 100 μg to 10 mg, 500 μg to 10 mg, 1 mg to 500 mg, 5 mg to 10 mg, 0.1 ng to 5 mg, 0.5 ng to 5 mg, 1 ng to 5 mg, 5 ng to 5 mg, 10 ng to 5 mg, 50 ng to 5 mg, 100 ng to 5 mg, 500 ng to 500 mg, 1 μg to 5 mg, 5 μg to 5 mg, 10 μg to 5 mg, 50 μg to 5 mg, 100 μg to 5 mg, 500 μg to 5 mg, 1 mg to 500 mg, 0.1 ng to 1 mg, 0.5 ng to 1 mg, 1 ng to 1 mg, 5 ng to 1 mg, 10 ng to 1 mg, 50 ng to 1 mg, 100 ng to 1 mg, 500 ng to 500 mg, 1 μg to 1 mg, 5 μg to 1 mg, 10 μg to 1 mg, 50 μg to 1 mg, 100 μg to 1 mg, 500 μg to 1 mg, 0.1 ng to 500 μg, 0.5 ng to 500 μg, 1 ng to 500 μg, 5 ng to 500 μg, 10 ng to 500 μg, 50 ng to 500 μg, 100 ng to 500 μg, 500 ng to 500 μg, 1 μg to 500 μg, 5 μg to 500 μg, 10 μg to 500 μg, 50 μg to 500 μg, 100 μg to 500 μg, 0.1 ng to 100 μg, 0.5 ng to 100 μg, 1 ng to 100 μg, 5 ng to 100 μg, 10 ng to 100 μg, 50 ng to 100 μg, 100 ng to 100 μg, 500 ng to 100 μg, 1 μg to 100 μg, 5 μg to 100 μg, 10 μg to 100 μg, 50 μg to 100 μg, 0.1 ng to 50 μg, 0.5 ng to 50 μg, 1 ng to 50 μg, 5 ng to 50 μg, 10 ng to 50 μg, 50 ng to 50 μg, 100 ng to 50 μg, 500 ng to 50 μg, 1 μg to 50 μg, 5 μg to 50 μg, 10 μg to 50 μg, 0.1 ng to 10 μg, 0.5 ng to 10 μg, 1 ng to 10 μg, 5 ng to 10 μg, 10 ng to 10 μg, 50 ng to 10 μg, 100 ng to 10 μg, 500 ng to 10 μg, 1 μg to 10 μg, 5 μg to 10 μg, 0.1 ng to 5 μg, 0.5 ng to 5 μg, 1 ng to 5 μg, 5 ng to 5 μg, 10 ng to 5 μg, 50 ng to 5 μg, 100 ng to 5 μg, 500 ng to 5 μg, 1 μg to 5 μg, 0.1 ng to 1 μg, 0.5 ng to 1 μg, 1 ng to 1 μg, 5 ng to 1 μg, 10 ng to 1 μg, 50 ng to 1 μg, 100 ng to 1 μg, 500 ng to 1 μg, 0.1 ng to 500 ng, 0.5 ng to 500 ng, 1 ng to 500 ng, 5 ng to 500 ng, 10 ng to 500 ng, 50 ng to 500 ng, 100 ng to 500 ng, 0.1 ng to 100 ng, 0.5 ng to 100 ng, 1 ng to 100 ng, 5 ng to 100 ng, 10 ng to 100 ng, 50 ng to 100 ng, 0.1 ng to 50 ng, 0.5 ng to 50 ng, 1 ng to 50 ng, 5 ng to 50 ng, 10 ng to 50 ng, 0.1 ng to 10 ng, 0.5 ng to 10 ng, 1 ng to 10 ng, 5 ng to 10 ng, 0.1 ng to 5 ng, 0.5 ng to 5 ng, 1 ng to 5 ng, 0.1 ng to 1 ng, 0.1 ng to 1 ng, or 0.1 ng to 0.5 ng.

In other embodiments, a microbial community, e.g., a high-complexity defined gut microbial community of the present invention is consumed at a rate of 1×10⁶ to 1×10¹³ CFUs a day, 1×10⁶ to 1×10¹² CFUs a day, 1×10⁶ to 1×10¹¹ CFUs a day, 1×10⁶ to 1×10¹⁰ CFUs a day, 1×10⁶ to 1×10⁹ CFUs a day, 1×10⁶ to 1×10⁸ CFUs a day, 1×10⁶ to 1×10⁷ CFUs a day, 5×10⁶ to 1×10¹³ CFUs a day, 5×10⁶ to 1×10¹² CFUs a day, 5×10⁶ to 1×10¹¹ CFUs a day, 5×10⁶ to 1×10¹⁰ CFUs a day, 5×10⁶ to 1×10⁹ CFUs a day, 5×10⁶ to 1×10⁸ CFUs a day, 5×10⁶ to 1×10⁷ CFUs a day, 1×10⁷ to 1×10¹³ CFUs a day, 1×10⁷ to 1×10¹² CFUs a day, 1×10⁷ to 1×10¹¹ CFUs a day, 1×10⁷ to 1×10¹⁰ CFUs a day, 1×10⁷ to 1×10⁹ CFUs a day, 1×10⁷ to 1×10⁸ CFUs a day, 5×10⁷ to 1×10¹³ CFUs a day, 5×10⁷ to 1×10¹² CFUs a day, 5×10⁷ to 1×10¹¹ CFUs a day, 5×10⁷ to 1×10¹⁰ CFUs a day, 5×10⁷ to 1×10⁹ CFUs a day, 5×10⁷ to 1×10⁸ CFUs a day, 1×10⁸ to 1×10¹³ CFUs a day, 1×10⁸ to 1×10¹² CFUs a day, 1×10⁸ to 1×10¹¹ CFUs a day, 1×10⁸ to 1×10¹⁰ CFUs a day, 1×10⁸ to 1×10⁹ CFUs a day, 5×10⁸ to 1×10¹³ CFUs a day, 5×10⁸ to 1×10¹² CFUs a day, 5×10⁸ to 1×10¹¹ CFUs a day, 5×10⁸ to 1×10¹⁰ CFUs a day, 5×10⁸ to 1×10⁹ CFUs a day, 1×10⁹ to 1×10¹³ CFUs a day, 1×10⁹ to 1×10¹² CFUs a day, 1×10⁹ to 1×10¹¹ CFUs a day, 1×10⁹ to 1×10¹⁰ CFUs a day, 5×10⁹ to 1×10¹³ CFUs a day, 5×10⁹ to 1×10¹² CFUs a day, 5×10⁹ to 1×10¹¹ CFUs a day, 5×10⁹ to 1×10¹⁰ CFUs a day, 1×10¹⁰ to 1×10¹³ CFUs a day, 1×10¹⁰ to 1×10¹² CFUs a day, 1×10¹⁰ to 1×10¹¹ CFUs a day, 5×10¹⁰ to 1×10¹³ CFUs a day, 5×10¹⁰ to 1×10¹² CFUs a day or 5×10¹⁰ to 1×10¹¹ CFUs a day.

In other embodiments, a microbial community, e.g., a high-complexity defined gut microbial community of the present invention is consumed at a rate of 0.1 ng to 500 mg a day, 0.5 ng to 500 mg a day, 1 ng to 500 mg a day, 5 ng to 500 mg a day, 10 ng to 500 mg a day, 50 ng to 500 mg a day, 100 ng to 500 mg a day, 500 ng to 500 mg a day, 1 μg to 500 mg a day, 5 μg to 500 mg a day, 10 μg to 500 mg a day, 50 μg to 500 mg a day, 100 μg to 500 mg a day, 500 μg to 500 mg a day, 1 mg to 500 mg a day, 5 mg to 500 mg a day, 10 mg to 500 mg a day, 50 mg to 500 mg a day, 100 mg to 500 mg a day, 0.1 ng to 100 mg a day, 0.5 ng to 100 mg a day, 1 ng to 100 mg a day, 5 ng to 100 mg a day, 10 ng to 100 mg a day, 50 ng to 100 mg a day, 100 ng to 100 mg a day, 500 ng to 500 mg a day, 1 μg to 100 mg a day, 5 μg to 100 mg a day, 10 μg to 100 mg a day, 50 μg to 100 mg a day, 100 μg to 100 mg a day, 500 μg to 100 mg a day, 1 mg to 500 mg a day, 5 mg to 100 mg a day, 10 mg to 100 mg a day, 50 mg to 100 mg a day, 0.1 ng to 50 mg a day, 0.5 ng to 50 mg a day, 1 ng to 50 mg a day, 5 ng to 50 mg a day, 10 ng to 50 mg a day, 50 ng to 50 mg a day, 100 ng to 50 mg a day, 500 ng to 500 mg a day, 1 μg to 50 mg a day, 5 μg to 50 mg a day, 10 μg to 50 mg a day, 50 μg to 50 mg a day, 100 μg to 50 mg a day, 500 μg to 50 mg a day, 1 mg to 500 mg a day, 5 mg to 50 mg a day, 10 mg to 50 mg a day, 0.1 ng to 10 mg a day, 0.5 ng to 10 mg a day, 1 ng to 10 mg a day, 5 ng to 10 mg a day, 10 ng to 10 mg a day, 50 ng to 10 mg a day, 100 ng to 10 mg a day, 500 ng to 500 mg a day, 1 μg to 10 mg a day, 5 μg to 10 mg a day, 10 μg to 10 mg a day, 50 μg to 10 mg a day, 100 μg to 10 mg a day, 500 μg to 10 mg a day, 1 mg to 500 mg a day, 5 mg to 10 mg a day, 0.1 ng to 5 mg a day, 0.5 ng to 5 mg a day, 1 ng to 5 mg a day, 5 ng to 5 mg a day, 10 ng to 5 mg a day, 50 ng to 5 mg a day, 100 ng to 5 mg a day, 500 ng to 500 mg a day, 1 μg to 5 mg a day, 5 μg to 5 mg a day, 10 μg to 5 mg a day, 50 μg to 5 mg a day, 100 μg to 5 mg a day, 500 μg to 5 mg a day, 1 mg to 500 mg a day, 0.1 ng to 1 mg a day, 0.5 ng to 1 mg a day, 1 ng to 1 mg a day, 5 ng to 1 mg a day, 10 ng to 1 mg a day, 50 ng to 1 mg a day, 100 ng to 1 mg a day, 500 ng to 500 mg a day, 1 μg to 1 mg a day, 5 μg to 1 mg a day, 10 μg to 1 mg a day, 50 μg to 1 mg a day, 100 μg to 1 mg a day, 500 μg to 1 mg a day, 0.1 ng to 500 μg a day, 0.5 ng to 500 μg a day, 1 ng to 500 μg a day, 5 ng to 500 μg a day, 10 ng to 500 μg a day, 50 ng to 500 μg a day, 100 ng to 500 μg a day, 500 ng to 500 μg a day, 1 μg to 500 μg a day, 5 μg to 500 μg a day, 10 μg to 500 μg a day, 50 μg to 500 μg a day, 100 μg to 500 μg a day, 0.1 ng to 100 μg a day, 0.5 ng to 100 μg a day, 1 ng to 100 μg a day, 5 ng to 100 μg a day, 10 ng to 100 μg a day, 50 ng to 100 μg a day, 100 ng to 100 μg a day, 500 ng to 100 μg a day, 1 μg to 100 μg a day, 5 μg to 100 μg a day, 10 μg to 100 μg a day, 50 μg to 100 μg a day, 0.1 ng to 50 μg a day, 0.5 ng to 50 μg a day, 1 ng to 50 μg a day, 5 ng to 50 μg a day, 10 ng to 50 μg a day, 50 ng to 50 μg a day, 100 ng to 50 μg a day, 500 ng to 50 μg a day, 1 μg to 50 μg a day, 5 μg to 50 μg a day, 10 μg to 50 μg a day, 0.1 ng to 10 μg a day, 0.5 ng to 10 μg a day, 1 ng to 10 μg a day, 5 ng to 10 μg a day, 10 ng to 10 μg a day, 50 ng to 10 μg a day, 100 ng to 10 μg a day, 500 ng to 10 μg a day, 1 μg to 10 μg a day, 5 μg to 10 μg a day, 0.1 ng to 5 μg a day, 0.5 ng to 5 μg a day, 1 ng to 5 μg a day, 5 ng to 5 μg a day, 10 ng to 5 μg a day, 50 ng to 5 μg a day, 100 ng to 5 μg a day, 500 ng to 5 μg a day, 1 μg to 5 μg a day, 0.1 ng to 1 μg a day, 0.5 ng to 1 μg a day, 1 ng to 1 μg a day, 5 ng to 1 μg a day, 10 ng to 1 μg a day, 50 ng to 1 μg a day, 100 ng to 1 μg a day, 500 ng to 1 μg a day, 0.1 ng to 500 ng a day, 0.5 ng to 500 ng a day, 1 ng to 500 ng a day, 5 ng to 500 ng a day, 10 ng to 500 ng a day, 50 ng to 500 ng a day, 100 ng to 500 ng a day, 0.1 ng to 100 ng a day, 0.5 ng to 100 ng a day, 1 ng to 100 ng a day, 5 ng to 100 ng a day, 10 ng to 100 ng a day, 50 ng to 100 ng a day, 0.1 ng to 50 ng a day, 0.5 ng to 50 ng a day, 1 ng to 50 ng a day, 5 ng to 50 ng a day, 10 ng to 50 ng a day, 0.1 ng to 10 ng a day, 0.5 ng to 10 ng a day, 1 ng to 10 ng a day, 5 ng to 10 ng a day, 0.1 ng to 5 ng a day, 0.5 ng to 5 ng a day, 1 ng to 5 ng a day, 0.1 ng to 1 ng a day, 0.1 ng to 1 ng a day, or 0.1 ng to 0.5 ng a day.

In some embodiments, the microbial composition of the present invention is administered for a period of at least 1 day to 1 week, 1 week to 1 month, 1 month to 3 months, 3 months to 6 months, 6 months to 1 year, or more than 1 year. For example, in some embodiments, the microbial composition of the present invention is administered for a period of at least 1 day, 2 days, 3 days, 4 days, 5 days, 6 days, 1 week, 2 weeks, 3 weeks, 1 month, 2 months, 3 months, 4 months, 5 months, 6 months, or 1 year.

In some embodiments, a microbial community, e.g., a high-complexity defined gut microbial community of the present invention is administered as a single dose or as multiple doses. For example, in some embodiments, a microbial community, e.g., a high-complexity defined gut microbial community of the present invention, is administered once a day for 2 days, 3 days, 4 days, 5 days, 6 days, 1 week, 2 weeks 3 weeks, 1 month, 2 months, 3 months, 4 months, 5 months, 6 months, or 1 year. In some embodiments, a microbial community, e.g., a high-complexity defined gut microbial community of the present invention, is administered multiple times daily. For example, in some embodiments, a microbial community, e.g., a high-complexity defined gut microbial community of the present invention, is administered twice daily, three times daily, 4 times daily, or 5 times daily. In some embodiments, a microbial community, e.g., a high-complexity defined gut microbial community of the present invention, is administered intermittently. For example, in some embodiments, a microbial community, e.g., a high-complexity defined gut microbial community of the present invention is administered once weekly, once monthly, or when a subject is in need thereof.

### 12. Combination Therapy

In some embodiments, a microbial community, e.g., a high-complexity defined gut microbial community of the present invention, can be administered in combination with other agents. For example, in some embodiments, a microbial community, e.g., a high-complexity defined gut microbial community of the present invention, can be administered concurrently with or after an antimicrobial agent, an antifungal agent, an antiviral agent, an antiparasitic agent or a prebiotic. Administration may be sequential over a period of hours or days, or simultaneously.

For example, in some embodiments, a microbial community, e.g., a high-complexity defined gut microbial community, can be administered concurrently with or after one or more than one antibacterial agent selected from fluoroquinolone antibiotics (e.g., ciprofloxacin, levaquin, floxin, tequin, avelox, and norflox); cephalosporin antibiotics (e.g., cephalexin, cefuroxime, cefadroxil, cefazolin, cephalothin, cefaclor, cefamandole, cefoxitin, cefprozil, and ceftobiprole); penicillin antibiotics (e.g., amoxicillin, ampicillin, penicillin V, dicloxacillin, carbenicillin, vancomycin, and methicillin); tetracycline antibiotics (e.g., tetracycline, minocycline, oxytetracycline, and doxycycline); and carbapenem antibiotics (e.g., ertapenem, doripenem, imipenem/cilastatin, and meropenem).

For example, in some embodiments, a microbial community, e.g., a high-complexity defined gut microbial community, can be administered concurrently with or after one or more than one antiviral agent selected from Abacavir, Acyclovir, Adefovir, Amprenavir, Atazanavir, Cidofovir, Darunavir, Delavirdine, Didanosine, Docosanol, Efavirenz, Elvitegravir, Emtricitabine, Enfuviltide, Etravirine, Famciclovir, Foscamet, Fomivirsen, Ganciclovir, Indinavir, Idoxuridine, Lamivudine, Lopinavir Maraviroc, MK-2048, Nelfinavir, Nevirapine, Penciclovir, Raltegravir, Rilpivirine, Ritonavir, Saquinavir, Stavudine, Tenofovir Trifluridine, Valaciclovir, Valganciclovir, Vidarabine, Ibacitabine, Amantadine, Oseltamivir, Rimantidine, Tipranavir, Zalcitabine, Zanamivir, and Zidovudine.

In some embodiments, a microbial community, e.g., a high-complexity defined gut microbial community can be administered concurrently with or after one or more than one antifungal agent selected from miconazole, ketoconazole, clotrimazole, econazole, omoconazole, bifonazole, butoconazole, fenticonazole, isoconazole, oxiconazole, sertaconazole, sulconazole, and tioconazole; triazole antifungals such as fluconazole, itraconazole, isavuconazole, ravuconazole, posaconazole, voriconazok, terconazole, and albaconazole; thiazole antifungals such as abafungin; allylamine antifungals such as terbinafine, naftifine, and butenafine; and echinocandin antifungals such as anidulafungin, caspofungin, and micafungin; polygodial; benzoic acid; ciclopirox; tolnaftate; undecylenic acid; flucytosine or 5-fluorocytosine; griseofulvin; and haloprogin.

In some embodiments, a microbial community, e.g., a high-complexity defined gut microbial community, can be administered concurrently with or after one or more than one anti-inflammatory and/or immunosuppressive agent selected from corticosteroids, mesalazine, mesalamine, sulfasalazine, sulfasalazine derivatives, cyclosporin A, mercaptopurine, azathiopurine, prednisone, methotrexate, antihistamines, glucocorticoids, epinephrine, theophylline, cromolyn sodium, anti-leukotrienes, anticholinergics, monoclonal anti-IgE, antibodies, and vaccines.

In some embodiments, a microbial community, e.g., a high-complexity defined gut microbial community of the present invention, can be administered concurrently with or after one or more than one prebiotic selected from, but not limited to, amino acids, biotin, fructooligosaccharides, galactooligosaccharides, inulin, lactulose, mannan oligosaccharides, oligofructose-enriched inulin, oligofructose, oligodextrose, tagatose, trans-galactooligosaccharide, and xylooligosaccharides.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1: Sourcing and Identification of Active and Supportive Microbial Strains

Microbial strains were purchased from a depository (e.g., the American Type Culture Collection (ATCC)) or are derived from human donor fecal samples.

Microbial strains purchased from a depository were cultured according to depository instructions and using the media as described in Table 11.

TABLE 11
Microbial Strains and Culture Media

| | Media |
|---|---|
| | (see Tables |
| Strain | 1-6 above) |
| Acidaminococcus fermentans—VR4 | B |
| Acidaminococcus sp.—D21 | B |
| Adlercreutzia equolifaciens—FJC-B9 | C2 + L- |
| | Arginine |
| Alistipes finegoldii—AHN 2437 | C2 |
| Alistipes onderdonkii—WAL 8169 | B |
| Anaerofustis stercorihominis—ATCC BAA-858, CCUG | F |
| 47767, CIP 108481, WAL 14563 | |
| Bilophila wadsworthia—WAL 7959 [Lab 88-130H] | B |
| Blautia hansenii—VPI C7-24 | B |
| Blautia hydrogenotrophica—S5a33 | C2 |
| Blautia obeum—ATCC 29174, KCTC 15206, VPI B3-21 | B |
| Catenibacterium mitsuokai—RCA14-39, CIP 106738, JCM | B |
| 10609 | |
| Clostridium asparagiforme—N6, CCUG 48471 | C2 |
| Collinsella stercoris—RCA 55-54, JCM 10641 | C2 |
| Coprococcus eutactus—VPI C33-22 | B |
| Parabacteroides distasonis—NCTC 11152 | B |
| Holdemania filiformis—VPI J1-31B-1, ATCC 51649 | B |
| Hungatella hathewayi—1313, CCUG 43506, CIP 109440, | B |
| MTCC 10951 | |
| Intestinimonas butyriciproducens—SRB-521-5-1, CCUG 63529 | B |
| Solobacterium moorei—RCA59-74, CIP 106864, JCM 10645 | B |
| Olsenellauli—D76D-27C, ATCC 49627, CIP 109912 | B |
| Roseburia inulinivorans—A2-194, CIP 109405, JCM 17584, | B |
| NCIMB 14030 | |
| Alistipes putredinis—CCUG 45780, CIP 104286, ATCC | B |
| 29800, Carlier 10203, VPI 3293 | |
| Clostridium spiroforme—VPI C28-23-1A, ATCC 29900, | B |
| NCTC 11211 | |
| Slackia exigua—S-7, ATCC 700122, JCM 11022, KCTC 5966 | B |
| Bacteroides pectinophilus—N3 | B |
| Butyrivibrio crossotus—T9-40A, ATCC 29175 | B |
| Subdoligranulum variabile—BI-114, CCUG 47106 | YCFAC |
| Turicibacter sanguinis—MOL361, NCCB 100008 | B |
| Streptococcus salivarius subsp. therm ophilus—LMD-9 | B |
| Oscillibacter sp.—KLE 1728 | D |
| Desulfovibrio piger—VPIC3-23 [DSM 749] | B |
| Lactobacillus ruminis—E 194e | B |
| Clostridium hiranonis—TO-931, JCM 10541, KCTC 15199 | B |
| Clostridium sp.—L2-50 | B |
| Clostridium orbiscindens—1_3_50AFAA | B |
| Alistipes shahii—WAL 8301 | B |
| Faecalibacterium prausnitzii—A2-165, JCM 31915 | B |
| Akkermansia muciniphila—Muc [CIP 107961] | A |
| Alistipes indistinctus—JCM 16068, YIT 12060 | A |
| Anaerobutyricum hallii—VPI B4-27 | A |
| Anaerostipes caccae—L1-92 | A |
| Anaerotruncus colihominis—277 | A |
| Bacteroides caccae—VPI 3452A [CIP 104201T, JCM 9498] | A |
| Bacteroides cellulosilyticus—CRE21, CCUG 44979 | A |
| Bacteroides coprocola—M16 | A |
| Bacteroides coprophilus—CB42, JCM 13818 | A |
| Bacteroides dorei—175 | A |
| Bacteroides dorei—5_1_36/D4 | A |
| Bacteroides eggerthii—ATCC 27754, NCTC 11155 | A |
| Bacteroides finegoldii—199 | A |
| Bacteroides fragilis—3_1_12 | A |
| Bacteroides intestinalis—341 | A |
| Bacteroides ovatus—NCTC 11153 | A |
| Bacteroides rodentium—ST28, CCUG 59334, JCM 16469 | A |
| Bacteroides thetaiotaomicron—1_1_6 | A |
| Bacteroides fragilis—2_1_16 | A |
| Bacteroides xylanisolvens—2_1_22 | A |
| Parabacteroides distasonis—3_1_19 | A |
| Bacteroides dorea—9_1_42FAA | A |
| Bacteroides ovatus—D2 | A |
| Bacteroides stercoris—VPI B3-21, ATCC 43183, CIP 104203, | A |
| JCM 9496 | |
| Bacteroides thetaiotaomicron—VPI 5482 [CIP 104206T, E50, | A |
| NCTC 10582] | |
| Bacteroides uniformis—ATCC 8492 | A |
| Bacteroides vulgatus—NCTC 11154 | A |
| Bifidobacterium pseudocatenulatum—B1279, ATCC 27919 | A |
| Blautia sp.—KLE 1732 | A |
| Blautia wexlerae—ATCC BAA-1564, JCM 17041, KCTC | A |
| 5965, WAL 14507 | |
| Clostridium hylemonae—TN-271, JCM 10539 | A |
| Clostridium leptum—VPI T7-24-1, ATCC 29065 | A |
| Tyzzerella nexilis DSM 1787 | A |
| Clostridium saccharolyticum—WM1, ATCC 35040, NRC 2533 | A |
| Absiella dolichum DSM 3991 | A |
| Collinsella aerofaciens—VPI 1003 [DSM 3979, JCM 10188] | A |
| Coprococcus comes—VPI CI-38 | A |
| Dialister invisus—E7.25, CCUG 47026 | A |
| Eubacterium rectale—VPI 0990 [CIP 105953] | A |
| Eubacterium siraeum—VPI T9-50-2, ATCC 29066, DSM 3996 | A |
| Eubacterium ventriosum—VPI 1013B | A |
| Holdemanella biformis—VPI C17-5, ATCC 27806, KCTC 5969 | A |
| Intestinibacter bartlettii—WAL 16138, ATCC BAA-827, | A |
| CCUG 48940 | |
| Megasphaera sp.—Sanger 24, Sanger_24 | A |
| Odoribacter splanchnicus—1651/6, ATCC 29572, CCUG | A |
| 21054, CIP 104287, LMG 8202, NCTC 10825 | |
| Parabacteroides merdae—VPI T4-1, ATCC 43184, CCUG | A |
| 38734, CIP 104202, JCM 9497 | |
| Parabacteroides sp.—D13 | A |
| Granulicatella adiacens—GaD [CIP 103243, DSM 9848] | A |
| Mitsuokella multacida—A 405-1, ATCC 27723, NCTC 10934 | A |
| Parabacteroides johnsonii—M-165, CIP 109537, JCM 13406 | A |
| Prevotella buccalis—HS4, ATCC 35310, NCDO 2354 | A |
| Prevotella copri—CB7, JCM 13464 | A |
| Clostridium sp.—VPI C48-50 (unassigned Clostridiales) | A |
| Ruminococcus gauvreauii—CCRI-16110, CCUG 54292, JCM | A |
| 14987, NML 060141 | |
| Ruminococcus lactaris—VPI X6-29 | A |
| Ruminococcus torques—VPI B2-51 | A |
| Alistipes senegalensis—CSUR P150, JCM 32779, JC50 | A |
| Bifidobacterium breve—S1, ATCC 15700, NCTC 11815 | A |
| Bifidobacterium catenulatum—B669, ATCC 27539, CECT | A |
| 7362, CIP 104175, DSM 20103 | |
| Butyricimonas virosa—MT12, CCUG 56611, JCM 15149 | A |
| Dorea formicigenerans—VPI C8-13 [JCM 9500] | A |
| Bacteroides plebeius—M12 | A |
| Ruminococcus gnavus—VPI C7-9 | A |
| Clostridium sp.—M62/1 | A |
| Slackia heliotrinireducens—RHS 1, ATCC 29202, NCTC 11029 | A |
| Clostridium methylpentosum—R2, ATCC 43829 | A |
| Ethanoligenens harbinense—YUAN-3, CGMCC 1.5033, JCM | A |
| 12961 | |
| Marvinbryantia formatexigens—I-52, CCUG 46960 | A |
| Clostridium bolteae—WAL 16351, [CCUG 46953], ATCC | A |
| BAA-613, Song et al. 2003 | |
| Clostridium scindens—VPI 13733, ATCC 35704, 19 | A |
| Bacteroides xylanisolvens—XB1A, CCUG 53782 | A |

### Isolation of Donor-Derived Active and Supportive Microbial Strains

Fecal donors are selected based on multiple criteria, including a health and medical history questionnaire, physical exam, and blood and stool tests for assessing pathogen-free status. Stool samples from donors who do not meet the inclusion criteria based on any of the above-mentioned assessment are discarded from quarantine.

Donors provide a stool sample sealed in a plastic container. Upon collection, stool samples are immediately transferred to an anaerobic chamber (5% CO₂, 5% H₂, 90% N₂) within 15 minutes of collection.

Once transferred to the anaerobic chamber, the fresh stool sample is labeled, weighed, evaluated for anomalies (presence of urine, toilet paper, etc.), and scored according to the Bristol scale. A stool sample weighing less than 45 g, or that fails to conform to a Bristol Stool Scale type 2, 3, 4 or 5, is rejected. Stool samples that meet the acceptance criteria are processed and aliquoted. 45 g of the stool sample is transferred into a sterile container for specific pathogen testing. The remainder of the sample is aliquoted into cryovials containing sterile glycerol solution (about 2 g of sample per vial; 6 vials per stool sample). These vials are transferred from the anaerobic chamber to a −80° C. freezer for storage until shipping on dry ice.

Microbial strain isolation is performed by making serial dilution aliquots of the stool samples and plating on a variety of microbial cultivation media suitable for growth of anaerobes. Specific enrichment techniques are performed for species having particular metabolic capabilities, such as consumption and degradation of oxalate from culture media. Species-specific PCR assays are developed to identify and follow the presence of specific species in the stool samples, isolated colonies, or enrichment culture. When appropriate, the enrichment cultures are plated on appropriate agar media to generate isolated colonies of microbes. After incubation under anaerobic conditions, microbial colonies are picked and transferred to plates with appropriate culture media to isolate the desired strain away from any microbial contaminating strain, followed by anaerobic incubation.

To identify isolated microbial colonies to the species level, either Matrix-Assisted Laser Desorption/Ionization Time-Of-Flight Mass Spectrometry (MALDI-TOF MS), 16S rRNA sequencing, or whole genome sequencing will be used. Identified colonies belonging to species of interest will be re-plated on appropriate culture media and their identify reconfirmed by 16S sequencing prior to their liquid media propagation and storage at −80° C.

### DNA Extraction

DNA was extracted from fecal samples using a Qiagen DNeasy Power Soil Kit (Qiagen, Germantown, Md.) in accordance with the manufacturer's instructions. Alternative methods for extracting DNA from fecal samples are well-known and routinely practiced in the art (e.g., described by Sambrook and Russell, Molecular Cloning: A Laboratory Manual, 3d ed., 2001).

### Whole Genome Shotgun Sequencing

Sequencing of DNA samples is carried out using the TruSeq Nano DNA Library Preparation kit (Illumina, San Diego, Calif., US) and a NextSeq platform (Illumina, San Diego, Calif., US). In brief, sequencing libraries are prepared from DNA extracted from each sample. DNA is mechanically fragmented using an ultrasonicator. The fragmented DNA is subjected to end repair and size selection of fragments, adenylation of 3′ ends, linked with adaptors, and DNA fragments enriched according to the TruSeq Nano DNA Library Preparation kit manual (Illumina, San Diego, Calif., US). Samples were sequenced to generate 30-40 million paired-end reads of 75 bp length.

16S rRNA Sequencing

Microbial species identification by 16S rRNA sequencing is performed by a method as known by persons of skill in the art (see, for example, Turner et al., 1999, “Investigating Deep Phylogenetic Relationships among Cyanobacteria and Plastids by Small Subunit rRNA Sequence Analysis,” J Eukaryot Microbiol. 46:327-338; Shin et al., 2016, “Analysis of the mouse gut microbiome using full-length 16S rRNA amplicon sequencing,” Sci Rep. 6:29681.) For each microbial stain, at least 1300 bp of 16S rRNA sequence is obtained for species level identification.

### MALDI-TOF MS

Species level identification by MALDI-TOF MS of microbial strains is performed by a method as known by persons of skill in the art (see, for example, Seuylemezian et al., 2018, “Development of a Custom MALDI-TOF MS Database for Species-Level Identification of Bacterial Isolates Collected From Spacecraft and Associated Surfaces,” Front Micrbiol. 9:780.) In brief, spots of microbial isolates are transferred to a well of a 48-well or 96-well plate, layered with 1 μl of 70% formic acid and left to air dry. 1 μl of α-Cyano-4-hydroxycinnamic acid matrix in 50% acetonitrile-25% trifluoroacetic acid is layered on the sample and left to air dry. MALDI-TOF MS is performed using, for example, a microbflex LT bench-top mass spectrometry instrument (Bruker Daltonics, Billerica, Mass.). Processing of spectral data is performed, for example, using flexAnalysis software (Bruker Daltonics, Billerica, Mass.). At least 10 spectra are calculated for each isolate to create a main spectral profile, wherein each spectral line that constitutes the main spectral profile has a log score of greater than 2.7 and a peak frequency greater than 75%.

### Example 2—Preparation and Optimization of a High-Complexity Defined Gut Microbial Community

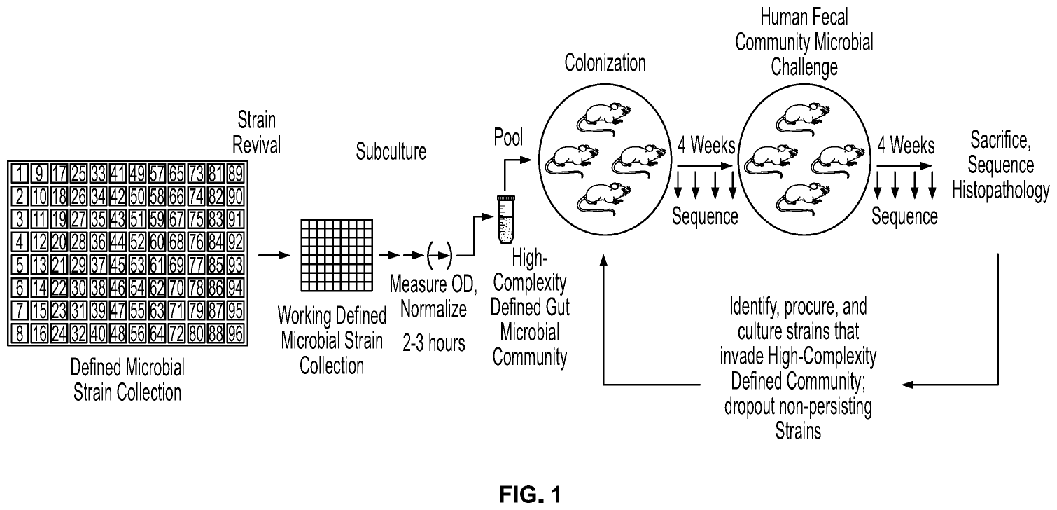

FIG. 1 shows a workflow schematic for the preparation and optimization of a high-complexity defined gut microbial community. Defined microbial strains purchased from American Type Culture Collection (ATCC, Manassas, Va.) were assembled as a frozen glycerol stock collection in 96-well plate format. Defined microbial strains were revived by culturing in 96-well plate format aliquots in growth medium and culture conditions in accordance with the supplier's instructions (“Working Defined Microbial Strain Collection). Defined microbial strains were sub-cultured for 24 hours, two times. Optical density of cultures was measured and cultures normalized to an O.D. value of 0.1. Defined microbial strains were pooled to form a high-complexity defined gut microbial community, washed and resuspended with PBS, then gavaged into gnotobiotic, 6-8 week old, female, Swiss Webster mice, once per day for 3 days, and permitted to colonize. Stool samples from inoculated mice were collected weekly for 4 consecutive weeks and frozen for subsequent DNA extraction and metagenomic analysis. 4-weeks after inoculation, mice were challenged with human fecal samples obtained from three donors. Human fecal samples were administered by oral gavage. Stool samples from challenged mice were collected weekly for 4 consecutive weeks and frozen for subsequent DNA extraction and metagenomic analysis. 4 weeks after human fecal microbial challenge, mice were sacrificed, and colon samples were prepared for histologic analysis. Strains identified to have “jumped in” to the community were identified (by metagenomic analysis), procured and cultured and optionally added to the high-complexity defined gut microbial community to produce a new high-complexity defined gut microbial community. Conversely, strains that were identified (by metagenomic analysis) to “drop out” of the community were omitted from the new high-complexity defined gut microbial community.

### DNA Extraction

DNA was extracted from fecal samples using a Qiagen DNesay Power Soil Kit (Qiagen, Germantown, Md.) in accordance with the manufacturer's instructions. Alternative methods for extracting DNA from fecal samples are well-known and routinely practiced in the art (e.g., described by Sambrook and Russell, Molecular Cloning: A Laboratory Manual, 3d ed., 2001).

### Metagenomic Analysis

Sequencing of the DNA samples was carried out using the TruSeq Nano DNA Library Preparation kit (Illumina, San Diego, Calif., US) and a NextSeq platform (Illumina, San Diego, Calif., US). In brief, sequencing libraries were prepared from DNA extracted from each sample. DNA was mechanically fragmented using an ultrasonicator. The fragmented DNA was subjected to end repair and size selection of fragments, adenylation of 3′ ends, linked with adaptors, and DNA fragments enriched according to the TruSeq Nano DNA Library Preparation kit manual (Illumina, San Diego, Calif., US). Samples were sequenced to generate 30-40 million paired-end reads of 75 bp length.

Each metagenome was run through the Metagenomic Intra-Species Diversity Analysis System (MIDAS) (see Nayfach et al., 2016, “An integrated metagenomics pipeline for strain profiling reveals novel patterns of bacterial transmission and biogeography,” Genome Res. 26 (11): 1612-1625.) which estimates the sequencing depth and relative abundance of each microbial species in a fecal sample by mapping reads to a reference database of 15 gene families of 5,952 bacterial species which each occur in nearly all bacterial genomes at one copy per genome.

### Backfill

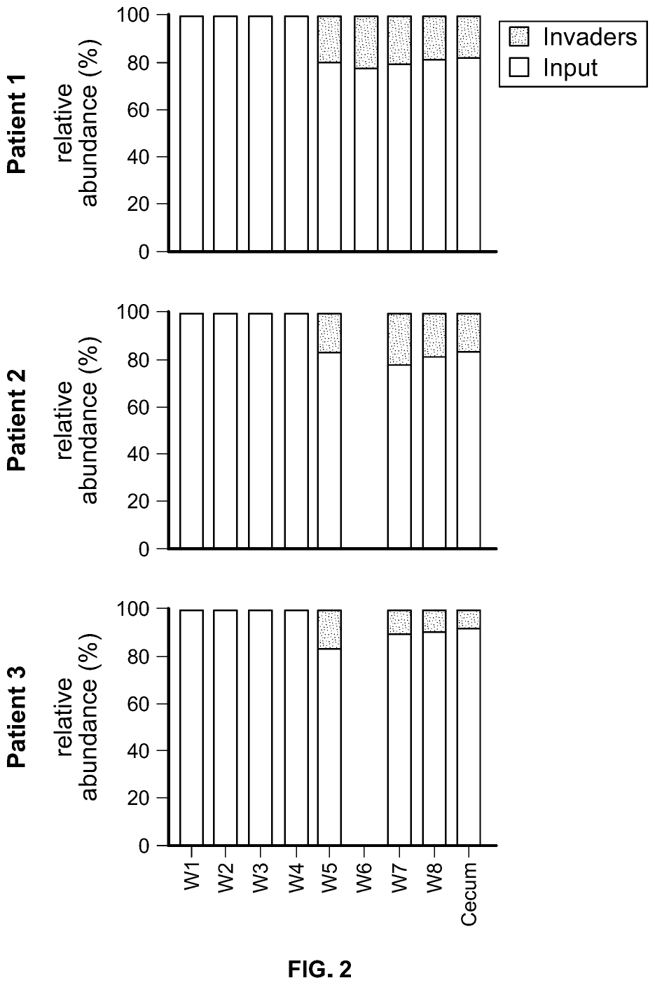

Defined microbial strains that did not engraft (i.e. dropped out) of the microbial community were identified by the metagenomic analysis above. Similarly, microbial strains from the human fecal microbial challenge that engrafted into the mouse gut (i.e. jumped in) were identified by the metagenomic analysis above. After a first human fecal microbial challenge, 97 defined microbial strains out of the inoculated 104 defined microbial strains persisted in fecal samples of the challenged mice and 7 defined microbial strains dropped out. In two mice, 26 microbial strains from the human fecal microbial challenge jumped in and in one mouse, 44 microbial strains from the human fecal microbial challenge jumped in. 22 of the 26 microbial strains that jumped into the microbial communities in two of the challenged mice were obtained from ATCC and added to the 97 defined microbial strains that persisted after human fecal microbial challenge to produce a high-complexity defined gut microbial community consisting of 119 defined microbial strains (See Table 12 and FIG. 2; “Invaders”=microbial strains that “dropped in” to community, “Input”=defined microbial strains inoculated into mouse).

TABLE 12

| | Defined Microbial | Defined | Human |
|---|---|---|---|
| | Strains Persisting | Microbial | Microbial |
| | Post-Microbial | Strains | Strains |
| Mouse | Challenge | Dropping Out | Jumping In |
| 1 | 97 | 7 | 26 |
| (Receiving Human | | | |
| Stool Sample 1) | | | |
| 2 | 97 | 7 | 26 |
| (Receiving Human | | | |
| Stool Sample 2) | | | |
| 3 | 97 | 7 | 44 |
| (Receiving Human | | | |
| Stool Sample 3) | | | |

### Example 3—Treatment of Mice with Persistent C. Difficile Infection

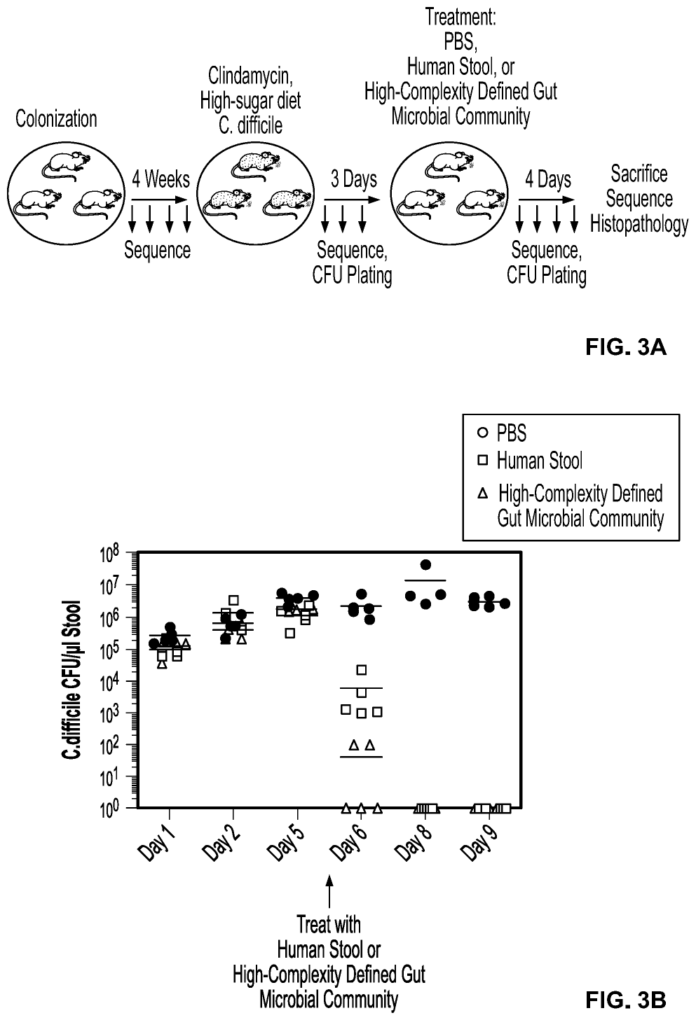

Gnotobiotic, 6-8 week old, female, Swiss Webster mice were colonized with human stool samples (200 μl of human stool diluted with an equal volume of PBS) by oral gavage. Stool samples from colonized mice were collected weekly for 4 consecutive weeks and frozen for subsequent DNA extraction and metagenomic analysis (as described in Example 2). 4 weeks following human fecal colonization, mice were treated with 200 μl of 1 mg/ml clindamycin by oral gavage. 24 hours after clindamycin treatment, mice were orally gavaged with 200 μl of turbid, overnight cultures of C. difficile, and maintained on a high-sugar diet. Stool samples from the inoculated mice were collected daily for 3 days post-inoculation for CFU plating and frozen for subsequent DNA extraction and metagenomic analysis. 3 days post-inoculation with C. difficile, mice were treated with human stool sample, the 119 strain high-complexity defined gut microbial community, or phosphate buffered saline (PBS) vehicle control. Stool samples from treated mice were collected daily for 4 days for CFU plating and frozen for subsequent DNA extraction and metagenomic analysis. 4 days post-treatment, mice were sacrificed, and colon samples (e.g., ceca) were prepared for mass spectrometry and histologic analysis. See FIG. 3A for schematic workflow of C. difficile infection and treatment schedule.

### CFU Plating

Stool samples were diluted in PBS, homogenized using a vortex mixer, and left to sediment. The supernatant was used to make serial 10-fold dilutions in PBS from 1×10⁻¹ to 10⁻⁵. A 100 μl aliquot of each dilution was plated onto CDDC selective agar (see, Table 13)

TABLE 13

| | Amount |
|---|---|
| Component | (in 500 mL) |
| C. difficile agar base | 34.5 g |
| Cysteine | 250 mg |
| Cefoxitin | 8 mg |
| D-cycloserine | 125 mg |
| Defibrinated horse blood | 35 ml |
| Milli-Q water (dHO)* | to total volume of 500 mL |

After 48 h of anaerobic incubation at 37° C., plates were inspected for growth of colonies with morphology characteristic of C. difficile. Plates with 30 to 300 colonies were counted with a detection limit of 3.0 log₁₀ CFU/g. For each dilution, the average of the two duplicate plates was calculated. When two successive dilutions yielded 30 to 300 colonies, the average count of both dilutions was calculated.

As shown in FIG. 3B, mice receiving treatment with human stool sample or the 119 defined microbial strain high-complexity defined gut microbial community, significantly reduced the number of C. difficile CFUs/μ1 in stool samples collected at 6 days post C. difficile infection (i.e. 3 days post treatment) as compared to mice treated with PBS alone.

### Example 4—Bile Acid Analysis by Mass Spectrometry

Frozen stool samples or homogenized cecum sections were pelleted in a centrifuge tube and extracted with ethyl acetate. Ethyl acetate was evaporated under vacuum and pellets were re-dissolved in 200 μl of 20% DMSO/MeOH.

LC-MS/MS was performed on an Agilent 6120 quadrupole mass spectrometer in negative mode using a Kinetex C18 stationary phase (1.7 μm) column.

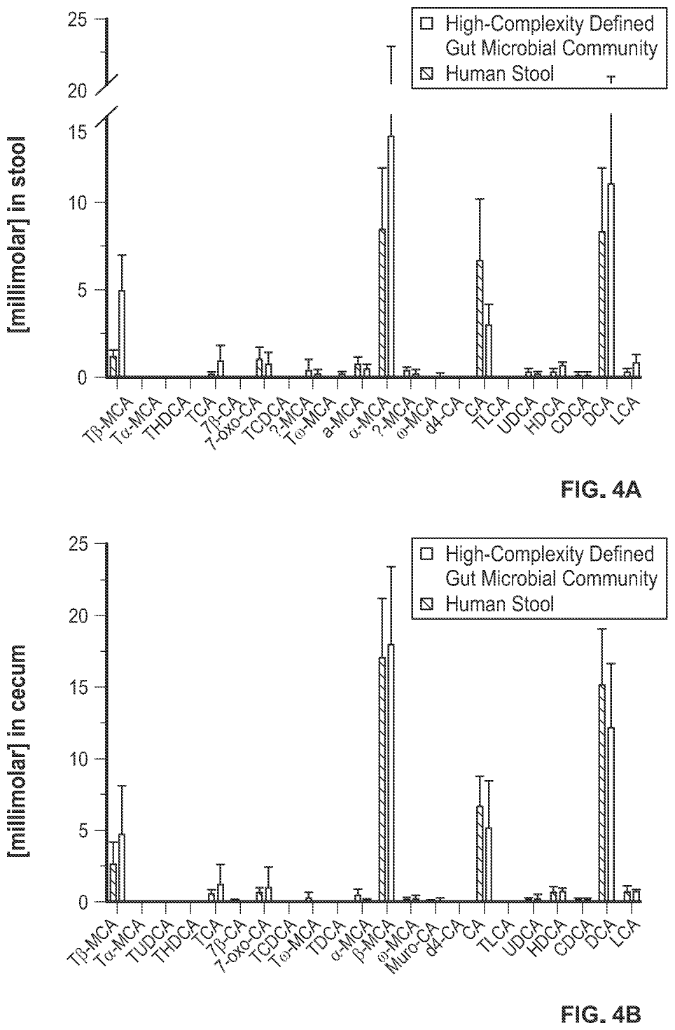

As shown in FIG. 4, bile acid concentrations in stool samples (FIG. 4A) and ceca homogenates (FIG. 4B) collected from mice treated with human stool sample and mice treated with the 119 defined microbial strain high-complexity defined gut microbial community had similar bile acid profiles and concentrations as quantified by MS.

### Example 5—Metabolite Analysis by Mass Spectrometry

Urine samples were thawed at room temperature and centrifuged at 13,000×g for 15 min at 4° C. to remove particulate matter. 2 volumes of ethyl acetate was added per volume of urine sample, and the solution was vortex mixed to precipitate proteins. Ethyl acetate was removed by rotary evaporation. Dried material was dissolved in 80% MeOH/DMSO and separated by reverse phase HPLC (Agilent 1200 series) for small molecule purification. NMR spectra were collected on either a Bruker Avance DRX500 or a Bruker AvanceIII 600-I spectrometer. Purification of the ethyl acetate fraction was carried on by gradient HPLC on a C18 reverse phase column.

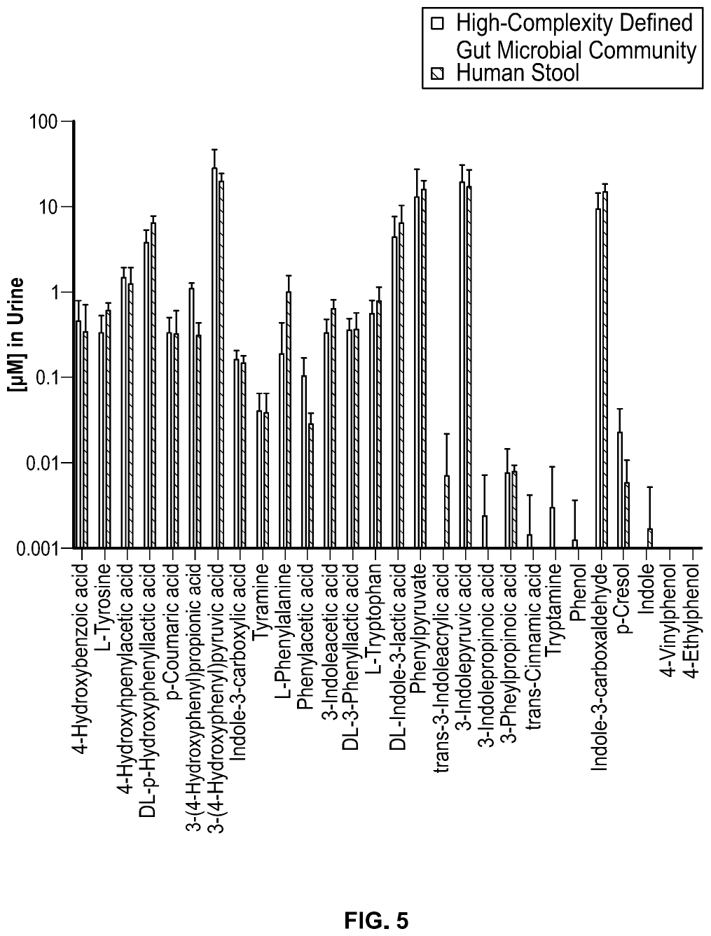

As shown in FIG. 5, urine samples collected from mice treated with human stool sample and mice treated with the 119 defined microbial strain high-complexity defined gut microbial community had similar bile acid profiles and concentrations as quantified by MS

### Example 6—Molecular Identification of Microbial Species

### Whole Genome Shotgun Sequencing

DNA extraction from isolated microbial cultures or fecal samples and whole genome shotgun sequencing is performed by methods as previously described in Example 2. Sequence reads are mapped against a comprehensive database of complete, sequenced genomes of all the defined microbial strains comprising a gut community.

16S rRNA Sequencing

Molecular identification by 16S rRNA sequencing of microbial colonies in liquid culture or resuspended in PBS is performed by a method as known by persons of skill in the art (see, for example, Turner et al., 1999, “Investigating Deep Phylogenetic Relationships among Cyanobacteria and Plastids by Small Subunit rRNA Sequence Analysis,” J Eukaryot Microbiol. 46:327-338; Shin et al., 2016, “Analysis of the mouse gut microbiome using full-length 16S rRNA amplicon sequencing,” Sci Rep. 6:29681.) For each defined microbial stain, at least 1300 bp of 16S rRNA sequence is obtained for species level identification.

### MALDI-TOF MS

Molecular identification by MALDI-TOF MS of microbial colonies in liquid culture or resuspended in PBS is performed by a method as known by persons of skill in the art (see, for example, Seuylemezian et al., 2018, “Development of a Custom MALDI-TOF MS Database for Species-Level Identification of Bacterial Isolates Collected From Spacecraft and Associated Surfaces,” Front Micrbiol. 9:780.) In brief, spots of microbial isolates are transferred to a well of a 48-well or 96-well plate, layered with 1 μl of 70% formic acid and left to air dry. 1 μl of α-Cyano-4-hydroxycinnamic acid matrix in 50% acetonitrile-25% trifluoroacetic acid is layered on the sample and left to air dry. MALDI-TOF MS is performed using, for example, a microbflex LT bench-top mass spectrometry instrument (Bruker Daltonics, Billerica, Mass.). Processing of spectral data is performed, for example, using flexAnalysis software (Bruker Daltonics, Billerica, Mass.). At least 10 spectra are calculated for each isolate to create a main spectral profile, wherein each spectral line that constitutes the main spectral profile has a log score of greater than 2.7 and a peak frequency greater than 75%.

### Example 7—Method of Treatment for Persistent C. difficile Infection

A high-complexity defined gut microbial community of the present invention is administered in an effective amount for the treatment of a persistent C. difficile infection in a mammalian subject in need thereof. The high-complexity defined gut microbial community is administered as a composition formulated for oral administration or other non-parenteral route of administration as described herein. The mammalian subject may or may not have been treated with antibiotics in advance of treatment with the high-complexity defined gut microbial community. The mammalian subject is treated once prior to improvement of symptoms associated with persistent C. difficile infection or a significant reduction in the number of C. difficile CFUs in the gut of the mammalian subject. Alternatively, the mammalian subject is treated two or more times prior to improvement of symptoms associated with persistent C. difficile infection or a significant reduction in the number of C. difficile CFUs in the gut of the mammalian subject.

### Example 8—Method of Treatment for Cholestatic Disease

A high-complexity defined gut microbial community of the present invention is administered in an effective amount for the treatment of a cholestatic disease in a mammalian subject in need thereof. The high-complexity defined gut microbial community is administered as a composition formulated for oral administration or other non-parenteral route of administration as described herein. The mammalian subject may or may not have been treated with antibiotics in advance of treatment with the high-complexity defined gut microbial community. The mammalian subject is treated once prior to improvement of symptoms associated with cholestatic disease or a significant modification in bile acid composition profile and/or concentrations in the gut of the mammalian subject. Alternatively, the mammalian subject is treated two or more times prior to improvement of symptoms associated with cholestatic disease or a significant modification in bile acid composition profile and/or concentrations in the gut of the mammalian subject.

### Example 9—Pathway-based Assembly of a High-complexity Defined Gut Microbial Community from Human Donor Fecal Samples

A high-complexity defined gut microbial community of the present invention is assembled by assignment of specific MetaCyc pathways to defined microbial stains.

Species-level compositional profiles of a donor fecal sample is generated using shotgun metagenomic sequencing.

A complete reference genome from the type-strain of every microbial species in the donor sample is retrieved and annotated using a custom computational pipeline that detects and accurately annotates MetaCyc pathways and the specific genes comprising those pathways. This annotation associates all metabolic pathways of interest with all the microbial strains in the fecal sample that utilize those pathways, thus defining a set of candidate microbes that can be isolated to cover/perform a desired metabolic function or fill a desired functional niche.

Having identified a set of microbial strains present in a fecal sample that utilizes a desired pathway, and given a set of metabolic pathways to be included in the high complexity-defined gut microbial community (i.e. core and substrate/metabolite panel pathways described above), a custom optimization algorithm is used to computationally design communities comprising microbes from donor samples that carry all, or substantially all, of the given set of metabolic pathways in addition to meeting the following criteria: (i) all metabolic pathways are utilized or encoded by at least three different species to incorporate functional redundancy; and (ii) at least three of the four major phyla in the normal human gut microbiome (Bacteroidetes, Actinobacteria, Firmicutes, Proteobacteria) are represented, and no one phylum accounts for more than 60% of the strains in the high-complexity defined gut microbial community (i.e. to capture the taxonomic diversity of the normal gut microbiome).

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A high-complexity defined gut microbial community, comprising:
a plurality of between 40 and 500 defined microbial strains, wherein the defined microbial strains comprise at least 3 of 4 phyla selected from the group consisting of Bacteroidetes, Firmicutes, Actinobacteria and Proteobacteria;
wherein the defined gut microbial community is capable of:
a. metabolizing at least 90% of enumerated substrates selected from the group consisting of: a-mannan (yeast), acetate, agarose, alanine, alginate, anthocyanin, arabinan, arabinogalactan, arabinoxylan, arginine, asparagine, Aspartate, b-glucans, butyrate, carrageenan, chitin, chlorogenic acids, chondroitin sulfate, cinnamic acid, Cysteine, dextran (40), Dihydrochalcones, Enterodiol, flavan-3-ols, flavanones, flavones, flavonols, folate, formate, galactomannan (carob), galacturonan (homo), galacturonate, glucomannan (konjac), glutamate, Glutamine, Glycine, Histidine, hyaluronan, hydrogen, hydroxycinnamic acids, hydroxyproline, inulin, isoflavones/isoflavanones, Isoleucine, lactate, laminarin, Leucine, levan, Lysine, Methionine, mucin O-linked glycans, Ornithine, Phenylalanine, porphyran, Proline, propionate, rhamnogalacturonan I, rhamnogalacturonan II, Secoisolariciresinol diglucoside, Serine, starch (potato), starch (structure 1), thiamine, Threonine, tryptophan, Tyrosine, Valine, xyloglucan, and xylooligosaccharides (XOS), and/or
b. producing at least 90% of enumerated metabolites selected from the group consisting of: formate, acetate, propionate, butyrate, isobutyrate, valerate, isovalerate, 2-methylbutyrate, caporate, isocaporate, 3-methylvaleric acid, L-phenylalanine, 3-phenylpropionic acid, phyenypyruvate, DL-3-phenyllactic acid, trans-cinnamic acid, phenyllactic acid, phenethylamine, L-tyrosine, 3-(4-hydroxyphenyl)propionic acid, 3-(4-hydroxyphenyl) pyruvic acid, DL-p-hydroxyphenyl lactic acid, p-coumaric acid, 4-hydroxyphenyl acetic acid, tyramine, phenol, p-cresol, 4-ethylphenol, 4-vinylphenol, 4-hydroxybenzoic acid, L-tryptophan, 3-indolepropionic acid, 3-indolepyruvic acid, DL-indole-3-lactic acid, trans-3-indoleacrylic acid, 3-indoleacetic acid, tryptamine, indole, skatol, indole-3-carboxylic acid, indole-3-carboxyaldehyde, N-acetyl-L-phenylalanine, phenylpropionylglycine, 3-(3-hydroxyphenyl) propionic acid, cinnamoylglycine, phenylacetylglycine, phenylacetylglutamine, hippuric acid, 2-hydroxyhippuric acid, 3-hydroxyhippuric acid, 4-hydroxyhippuric acid, 4-hydroxyphenylacetylglycine, phenyl sulfate, phenyl glucuronide, p-cresol sulfate, p-cresol glucuronide, 4-ethylphenol sulfate, 4-ethylphenol glucuronide, N-acetyl-L-tryptophan, 5-hydroxy-L-typtophan, N-acetyl serotonin, 3-indolepriopionylglycine, indolyl-3-acryloylglycine, indoxyl sulfate, indoxyl glucuronide, 5-hydroxyindole-3-acetic acid, indoleacetylglycine, lithocholic acid, murocholic acid, ursodeoxycholic acid, hyodeoxycholic acid, deoxycholic acid, 7-oxocholic acid, ω-muricholic acid, α-muricholic acid, β-muricholic acid, γ-muricholic acid, 7βcholic acid, taurolithocholic acid, tauroursodeoxycholic acid, taurohyodeoxycholic acid, taurochenodeoxycholic acid, taurodeoxycholic acid, tauro-β-muricholic acid, tauro-ω-muricholic acid, and taurocholic acid,
wherein the defined gut microbial community achieves substantial engraftment when administered to a gnotobiotic mouse; and
wherein the engrafted defined gut microbial community is stable following a human fecal community microbial challenge.

2. The high complexity defined gut microbial community of claim 1, wherein metabolization of a substrate and/or production of a metabolite can be determined by culturing the defined gut microbial community in vitro and measuring whether the substrate is metabolized and/or the metabolite is produced by liquid chromatography-mass spectrometry analysis.

3. The high complexity defined gut microbial community of claim 1 or 2, wherein metabolization of a substrate and/or production of a product can be determined by administering the defined gut microbial community to a gnotobiotic mouse and measuring whether the substrate is metabolized and/or the product is produced after a defined period of time by liquid chromatography-mass spectrometry analysis of a sample obtained from the mouse.

4. The high complexity defined gut microbial community of claim 3, wherein the defined gut microbial community is administered via a route selected from the group consisting of oral, rectal, fecal (by enema), and naso/oro-gastric gavage.

5. The high complexity defined gut microbial community of claim 3 or 4, wherein the defined period of time is about 1 hour, 2 hours, 4 hours, 8 hours, 12 hours, 24 hours, 2 days, 7 days, 14 days, 1 month, or 2 months.

6. The high complexity defined gut microbial community of any one of claims 3-5, wherein the sample is selected from the group consisting of a fecal sample, urine sample, blood sample, or serum sample.

7. A high-complexity defined gut microbial community, comprising:
a plurality of between 40 and 500 defined microbial strains, wherein the defined microbial strains comprise at least 3 of 4 phyla selected from the group consisting of Bacteroidetes, Firmicutes, Actinobacteria and Proteobacteria;
wherein the defined gut microbial community encodes the enzymes catalyzing all reactions at least 90% of the enumerated MetaCyc metabolic pathways selected from the group consisting of: 1CMET2-PWY, 2.6.1.32-RXN, AEROBACTINSYN-PWY, ALACAT2-PWY, ALADEG-PWY, ALANINE-DEG3-PWY, ALANINE-SYN2-PWY, ALANINE-VALINESYN-PWY, ANAPHENOXI-PWY, ARGASEDEG-PWY, ARGDEG-III-PWY, ARGDEG-IV-PWY, ARGDEGRAD-PWY, ARGDEG-V-PWY, ARG-GLU-PWY, ARGININE-SYN4-PWY, ARG-PRO-PWY, ARGSYNBSUB-PWY, ARGSYN-PWY, ASPARAGINE-BIOSYNTHESIS, ASPARAGINE-DEG1-PWY, ASPARAGINE-DEG1-PWY-1, ASPARAGINESYN-PWY, ASPARTATE-DEG1-PWY, ASPARTATESYN-PWY, ASPASN-PWY, ASPSYNII-PWY, AST-PWY, BETA-ALA-DEGRADATION-I-PWY, CAMALEXIN-SYN, CITRULBIO-PWY, CITRULLINE-DEG-PWY, COA-PWY, CODH-PWY, CYSTEINE-DEG-PWY, CYSTSYN-PWY, DAPLYSINESYN-PWY, ENTBACSYN-PWY, ETHYL-PWY, FAO-PWY, FERMENTATION-PWY, GLNSYN-PWY, GLUDEG-I-PWY, GLUGLNSYN-PWY, GLUTAMATE-DEG1-PWY, GLUTAMATE-SYN2-PWY, GLUTAMINDEG-PWY, GLUTAMINEFUM-PWY, GLUTATHIONESYN-PWY, GLUTDEG-PWY, GLUTORN-PWY, GLUTSYNIII-PWY, GLUTSYN-PWY, GLYCGREAT-PWY, GLYSYN-ALA-PWY, GLYSYN-PWY, GLYSYN-THR-PWY, HISDEG-PWY, HISHP-PWY, HISTDEG-PWY, HISTSYN-PWY, HOMOCYSDEGR-PWY, HOMOSER-METSYN-PWY, HOMOSERSYN-PWY, HSERMETANA-PWY, HYDROXYPRODEG-PWY, ILEUDEG-PWY, ILEUSYN-PWY, LARABITOLUTIL-PWY, LCYSDEG-PWY, LEU-DEG2-PWY, LEUSYN-PWY, LYSDEGII-PWY, LYSINE-AMINOAD-PWY, LYSINE-DEG1-PWY, MALATE-ASPARTATE-SHUTTLE-PWY, METH-ACETATE-PWY, METHANOGENESIS-PWY, METHIONINE-DEG1-PWY, MGLDLCTANA-PWY, ORN-AMINOPENTANOATE-CAT-PWY, ORNDEG-PWY, P101-PWY, P162-PWY, P163-PWY, P181-PWY, P261-PWY, P283-PWY, P401-PWY, P541-PWY, PHENYLALANINE-DEG1-PWY, PHESYN, PHO SLIP SYN2-PWY, PHOSPHONOTASE-PWY, PRO SYN-PWY, PROUT-PWY, PWY0-1021, PWY0-1221, PWY0-1299, PWY0-1303, PWY0-1305, PWY0-1313, PWY0-1317, PWY0-1321, PWY0-1338, PWY0-1347, PWY0-1355, PWY0-1356, PWY0-1534, PWY0-1544, PWY0-1565, PWY0-1576, PWY0-1577, PWY0-1578, PWY0-1585, PWY0-1601, PWY0-42, PWY0-461, PWY0-823, PWY0-901, PWY-1, PWY-1061, PWY-1121, PWY-1186, PWY1-2, PWY-1263, PWY-1622, PWY-1722, PWY-1781, PWY-181, PWY-1881, PWY-1962, PWY-1981, PWY1F-467, PWY1F-FLAVSYN, PWY1G-0, PWY-2021, PWY-2161, PWY-2181, PWY-2201, PWY-2821, PWY-2941, PWY-2942, PWY-3, PWY-3022, PWY-3081, PWY-3161, PWY-3162, PWY-3181, PWY-3301, PWY-3341, PWY-3385, PWY-3461, PWY-3462, PWY-3581, PWY-361, PWY-3661, PWY-3661-1, PWY-381, PWY-3841, PWY-3941, PWY3DJ-12, PWY3O-4107, PWY3O-4108, PWY-4, PWY-40, PWY-4002, PWY-4041, PWY-4201, PWY-4281, PWY-43, PWY-4321, PWY-4341, PWY-4361, PWY-46, PWY490-3, PWY490-4, PWY-4981, PWY-4983, PWY-4984, PWY4FS-6, PWY-5, PWY-5022, PWY-5024, PWY-5028, PWY-5029, PWY-5030, PWY-5031, PWY-5041, PWY-5048, PWY-5049, PWY-5057, PWY-5075, PWY-5076, PWY-5078, PWY-5079, PWY-5081, PWY-5082, PWY-5087, PWY-5097, PWY-5101, PWY-5103, PWY-5104, PWY-5108, PWY-5109, PWY-5129, PWY-5135, PWY-5136, PWY-5151, PWY-5154, PWY-5155, PWY-5159, PWY-5176, PWY-5188, PWY-5189, PWY-5196, PWY-5199, PWY-5207, PWY-5250, PWY-5254, PWY-5265, PWY-5280, PWY-5283, PWY-5290, PWY-5297, PWY-5298, PWY-5311, PWY-5314, PWY-5316, PWY-5319, PWY-5324, PWY-5329, PWY-5331, PWY-5332, PWY-5364, PWY-5381, PWY-5382, PWY-5386, PWY-5394, PWY-5399, PWY-5436, PWY-5437, PWY-5441, PWY-5443, PWY-5458, PWY-5467, PWY-5468, PWY-5473, PWY-5474, PWY-5494, PWY-5497, PWY-5499, PWY-5629, PWY-5651, PWY-5653, PWY-5665, PWY-5669, PWY-5675, PWY-5679, PWY-5686, PWY-5710, PWY-5736, PWY-5737, PWY-5739, PWY-5740, PWY-5742, PWY-5747, PWY-5748, PWY-5751, PWY-5754, PWY-5766, PWY-5770, PWY-5784, PWY-5788, PWY-5797, PWY-5800, PWY-581, PWY-5811, PWY-5818, PWY-5826, PWY-5877, PWY-5883, PWY-5886, PWY-5912, PWY-5913, PWY-5921, PWY-5936, PWY-5940, PWY-5958, PWY-5963, PWY-5968, PWY-5978, PWY-5980, PWY-5990, PWY-6003, PWY-6004, PWY-601, PWY-6030, PWY-6039, PWY-6045, PWY-6052, PWY-6053, PWY-6054, PWY-6055, PWY-6068, PWY-6069, PWY-6082, PWY-6120, PWY-6121, PWY-6122, PWY-6123, PWY-6124, PWY-6133, PWY-6134, PWY-6141, PWY-6143, PWY-6148, PWY-6151, PWY-6160, PWY-6173, PWY-6196, PWY-6219, PWY-622, PWY-6220, PWY-6233, PWY-6273, PWY-6277, PWY-6281, PWY-6307, PWY-6309, PWY-6313, PWY-6318, PWY-6320, PWY-6321, PWY-6322, PWY-6324, PWY-6328, PWY-6334, PWY-6339, PWY-6343, PWY-6344, PWY-6345, PWY-6346, PWY-6375, PWY-6376, PWY-6381, PWY-6386, PWY-6387, PWY-6397, PWY-6403, PWY-6407, PWY-6408, PWY-6409, PWY-6431, PWY-6435, PWY-6444, PWY-6455, PWY-6456, PWY-6457, PWY-6466, PWY-6471, PWY-6473, PWY-6481, PWY-6486, PWY-6493, PWY-6495, PWY-6511, PWY-6512, PWY-6519, PWY-6533, PWY-6535, PWY-6536, PWY-6537, PWY-6543, PWY-6549, PWY-6559, PWY-6562, PWY-6572, PWY-6573, PWY-6574, PWY-6578, PWY-6588, PWY-6614, PWY-6627, PWY66-301, PWY66-375, PWY-6638, PWY66-391, PWY-6642, PWY66-420, PWY66-421, PWY66-425, PWY66-426, PWY66-428, PWY-6643, PWY-6661, PWY-6673, PWY-6682, PWY-6690, PWY-6696, PWY-6711, PWY-6717, PWY-6720, PWY-6724, PWY-6728, PWY-6731, PWY-6735, PWY-6749, PWY-6769, PWY-6771, PWY-6772, PWY-6773, PWY-6781, PWY-6784, PWY-6790, PWY-6791, PWY-6802, PWY-6807, PWY-6808, PWY-6813, PWY-6815, PWY-6816, PWY-6817, PWY-6818, PWY-6821, PWY-6822, PWY-6823, PWY-6831, PWY-6832, PWY-6834, PWY-6840, PWY-6845, PWY-6853, PWY-6854, PWY-6855, PWY-6891, PWY-6892, PWY-6896, PWY-6898, PWY-6902, PWY-6907, PWY-6908, PWY-6920, PWY-6922, PWY-6936, PWY-6942, PWY-6949, PWY-6953, PWY-6955, PWY-6963, PWY-6964, PWY-6965, PWY-6968, PWY-6969, PWY-6981, PWY-6982, PWY-6986, PWY-6994, PWY-7000, PWY-701, PWY-7014, PWY-7015, PWY-7016, PWY-7018, PWY-7019, PWY-702, PWY-7022, PWY-7025, PWY-7028, PWY-7040, PWY-7046, PWY-7052, PWY-7054, PWY-7064, PWY-7072, PWY-7088, PWY-7090, PWY-7097, PWY-7104, PWY-7115, PWY-7117, PWY-7118, PWY-7126, PWY-7147, PWY-7153, PWY-7158, PWY-7176, PWY-7177, PWY-7185, PWY-7186, PWY-7219, PWY-7221, PWY-7234, PWY-7246, PWY-7248, PWY-7250, PWY-7255, PWY-7274, PWY-7275, PWY-7282, PWY-7288, PWY-7297, PWY-7304, PWY-7315, PWY-7316, PWY-7318, PWY-7342, PWY-7351, PWY-7356, PWY-7376, PWY-7377, PWY-7383, PWY-7387, PWY-7397, PWY-7398, PWY-7400, PWY-7414, PWY-7425, PWY-7430, PWY-7432, PWY-7440, PWY-7441, PWY-7456, PWY-7467, PWY-7498, PWY-7501, PWY-7506, PWY-7510, PWY-7514, PWY-7518, PWY-7520, PWY-7525, PWY-7531, PWY-7532, PWY-7533, PWY-7536, PWY-7542, PWY-7543, PWY-7547, PWY-7549, PWY-7550, PWY-7555, PWY-7561, PWY-7565, PWY-7570, PWY-7571, PWY-7600, PWY-7605, PWY-761, PWY-7612, PWY-7626, PWY-7645, PWY-7648, PWY-7649, PWY-7650, PWY-7665, PWY-7667, PWY-7668, PWY-7669, PWY-7671, PWY-7674, PWY-7688, PWY-7690, PWY-7693, PWY-7694, PWY-7701, PWY-7704, PWY-7706, PWY-7708, PWY-7717, PWY-7718, PWY-7719, PWY-7733, PWY-7734, PWY-7735, PWY-7737, PWY-7751, PWY-7761, PWY-7765, PWY-7767, PWY-7769, PWY-7770, PWY-7782, PWY-7790, PWY-7791, PWY-7793, PWY-7797, PWY-7811, PWY-7814, PWY-7822, PWY-7824, PWY-7826, PWY-7842, PWY-7850, PWY-7851, PWY-7855, PWY-7860, PWY-7861, PWY-7863, PWY-7867, PWY-7870, PWY-7880, PWY-7888, PWY-7889, PWY-7891, PWY-7892, PWY-7897, PWY-7901, PWY-7904, PWY-7907, PWY-7909, PWY-7910, PWY-7913, PWY-7917, PWY-7930, PWY-7931, PWY-7936, PWY-7953, PWY-7955, PWY-7956, PWY-7957, PWY-7958, PWY-7959, PWY-7960, PWY-7962, PWY-7977, PWY-7985, PWY-7986, PWY-7987, PWY-7988, PWY-7990, PWY-8002, PWY-8003, PWY-8006, PWY-8007, PWY-8008, PWY-8009, PWY-801, PWY-8010, PWY-8011, PWY-8013, PWY-8014, PWY-8015, PWY-8016, PWY-8017, PWY-8024, PWY-8032, PWY-8040, PWY-8043, PWY-8045, PWY-8071, PWY-8072, PWY-8080, PWY-8081, PWY-8082, PWY-8083, PWY-8086, PWY-8088, PWY-842, PWY-861, PWY-862, PWY8J2-1, PWY8J2-22, PWYDQC-4, PWYG-321, PWY-I9, PWYQT-4450, PWYQT-4476, PYRIDNUCSAL-PWY, PYRIDNUCSYN-PWY, PYRIDOXSYN-PWY, SAM-PWY, SERDEG-PWY, SERSYN-PWY, SPHINGOLIPID-SYN-PWY, TAURINEDEG-PWY, THRDLCTCAT-PWY, THREONINE-DEG2-PWY, TRNA-CHARGING-PWY, TRPCAT-PWY, TRPIAACAT-PWY, TRPKYNCAT-PWY, TRPSYN-PWY, TRYPDEG-PWY, TYRFUMCAT-PWY, TYRSYN, UDPNACETYLGALSYN-PWY, UDPNAGSYN-PWY, VALDEG-PWY, and VALSYN-PWY,
wherein the defined gut microbial community achieves substantial engraftment when administered to a gnotobiotic mouse; and
wherein the engrafted defined gut microbial community is stable following a human fecal community microbial challenge.

8. The high complexity defined gut microbial community of claim 7, wherein encoding the enzymes catalyzing all reactions of a MetaCyc metabolic pathway can be determined by culturing the defined gut microbial community in vitro and measuring whether a substrate in the pathway is metabolized, a metabolite in the pathway is produced, and/or a reaction intermediate in the pathway is produced by liquid chromatography-mass spectrometry analysis

9. The high complexity defined gut microbial community of claim 1 or 2, wherein encoding the enzymes catalyzing all reactions of a MetaCyc metabolic pathway can be determined by administering the defined gut microbial community to a gnotobiotic mouse and measuring whether a substrate in the pathway is metabolized, a metabolite in the pathway is produced, and/or a reaction intermediate in the pathway is produced after a defined period of time by liquid chromatography-mass spectrometry analysis of a sample obtained from the mouse.

10. The high complexity defined gut microbial community of claim 9, wherein the defined gut microbial community is administered via a route selected from the group consisting of oral, rectal, fecal (by enema), and naso/oro-gastric gavage.

11. The high complexity defined gut microbial community of claim 9 or 10, wherein the defined period of time is about 1 hour, 2 hours, 4 hours, 8 hours, 12 hours, 24 hours, 2 days, 7 days, 14 days, 1 month, or 2 months.

12. The high complexity defined gut microbial community of any one of claims 9-11, wherein the sample is selected from the group consisting of a fecal sample, urine sample, blood sample, or serum sample.

13. The high-complexity defined gut microbial community of any one of claims 1-12, wherein the at least 3 of 4 phyla comprise Bacteroidetes, Firmicutes, and Actinobacteria.

14. The high complexity defined gut microbial community of any one of claims 1-13, comprising Bacteroidetes, Firmicutes, Actinobacteria and Proteobacteria.

15. The high-complexity defined gut microbial community of any one of claims 1-14, wherein the defined microbial strains comprise phyla selected from the group consisting of Bateriodales, Clostridiales, Lactobacillales, Negativicutes, Eggerthellales, Bifidobacteriales, and Proteobacteria.

16. The high complexity defined gut microbial community of any one of claims 1-15, wherein the defined microbial strains comprise a genus selected from the group consisting of: Acidaminococcus, Adlercreutzia, Akkermansia, Alistipes, Anaerobutyricum, Anaerofustis, Anaerostipes, Anaerotruncus, Bacteroides, Parabacteroides, Bifidobacterium, Bilophila, Blautia, Catenibacterium, Clostridium, Tyzzerella, Absiella, Collinsella, Coprococcus, Dialister, Eubacterium, Holdemanella, Intestinibacter, Megasphaera, Odoribacter, Parabacteroides, Granulicatella, Holdemania, Hungatella, Intestinimonas, Solobacterium, Mitsuokella, Olsenella, Parabacteroides, Prevotella, Roseburia, Ruminococcus, Slackia, Butyrivibrio, Subdoligranulum, Turicibacter, Butyricimonas, Streptococcus, Dorea, Oscillibacter, Desulfovibrio, Ethanoligenens, Marvinbryantia, Lactobacillus, and Faecalibacterium.

17. The high complexity defined gut microbial community of any one of claims 1-16, wherein the defined microbial strains are selected from the group consisting of: Acidaminococcus fermentans, Acidaminococcus sp., Adlercreutzia equolifaciens, Akkermansia muciniphila, Alistipes finegoldii, Alistipes indistinctus, Alistipes onderdonkii, Anaerobutyricum hallii, Anaerofustis stercorihominis, Anaerostipes caccae, Anaerotruncus colihominis, Bacteroides caccae, Bacteroides cellulosilyticus, Bacteroides coprocola, Bacteroides coprophilus, Bacteroides dorei, Bacteroides eggerthii, Bacteroides finegoldii, Bacteroides fragilis, Bacteroides intestinalis, Bacteroides ovatus, Bacteroides rodentium, Bacteroides thetaiotaomicron, Bacteroides xylanisolvens, Parabacteroides distasonis, Bacteroides dorea, Bacteroides stercoris, Bacteroides uniformis, Bacteroides vulgatus, Bifidobacterium pseudocatenulatum, Bilophila wadsworthia, Blautia hansenii, Blautia hydrogenotrophica, Blautia obeum, Blautia sp., Blautia wexlerae, Catenibacterium mitsuokai, Clostridium asparagiforme, Clostridium hylemonae, Clostridium leptum, Tyzzerella nexilis, Clostridium saccharolyticum, Absiella dolichum, Collinsella aerofaciens, Collinsella stercoris, Coprococcus comes, Dialister invisus, Eubacterium rectale, Eubacterium siraeum, Eubacterium ventriosum, Coprococcus eutactus, Holdemanella biformis, Intestinibacter bartlettii, Megasphaera sp., Odoribacter splanchnicus, Parabacteroides merdae, Parabacteroides sp., Granulicatella adiacens, Holdemania filiformis, Hungatella hathewayi, Intestinimonas butyriciproducens, Solobacterium moorei, Mitsuokella multacida, Olsenella uli, Parabacteroides johnsonii, Prevotella buccalis, Prevotella copri, Roseburia inulinivorans, Clostridium sp., Ruminococcus gauvreauii, Ruminococcus lactaris, Ruminococcus torques, Alistipes putredinis, Alistipes senegalensis, Clostridium spiroforme, Slackia exigua, Bacteroides pectinophilus, Butyrivibrio crossotus, Subdoligranulum variabile, Turicibacter sanguinis, Bifidobacterium breve, Bifidobacterium catenulatum, Butyricimonas virosa, Streptococcus salivarius subsp. thermophilus, Dorea formicigenerans, Bacteroides plebeius, Ruminococcus gnavus, Oscillibacter sp., Clostridium sp., Slackia heliotrinireducens, Desulfovibrio piger, Clostridium methylpentosum, Ethanoligenens harbinense, Marvinbryantia formatexigens, Lactobacillus ruminis, Clostridium bolteae, Clostridium hiranonis, Clostridium scindens, Clostridium sp., Clostridium orbiscindens, Alistipes shahii, and Faecalibacterium prausnitzii.

18. The high complexity defined gut microbial community of any one of claims 1-17, wherein the defined microbial strains are selected from the group consisting of: Acidaminococcus fermentans—VR4, Acidaminococcus sp.—D21, Adlercreutzia equolifaciens—FJC-B9, Akkermansia muciniphila—Muc [CIP 107961], Alistipes finegoldii—AHN 2437, Alistipes indistinctus—JCM 16068, YIT 12060, Alistipes onderdonkii—WAL 8169, Anaerobutyricum hallii—VPI B4-27, Anaerofustis stercorihominis—ATCC BAA-858, CCUG 47767, CIP 108481, WAL 14563, Anaerostipes caccae—L1-92, Anaerotruncus colihominis—277, Bacteroides caccae—VPI 3452A [CIP 104201T, JCM 9498], Bacteroides cellulosilyticus—CRE21, CCUG 44979, Bacteroides coprocola—M16, Bacteroides coprophilus—CB42, JCM 13818, Bacteroides dorei —175, Bacteroides dorei —5_1_36/D4, Bacteroides eggerthii—ATCC 27754, NCTC 11155, Bacteroides finegoldii—199, Bacteroides fragilis—3_1_12, Bacteroides intestinalis—341, Bacteroides ovatus—NCTC 11153, Bacteroides rodentium—ST28, CCUG 59334, JCM 16469, Bacteroides thetaiotaomicron —1_1_6, Bacteroides fragilis—2_1_16, Bacteroides xylanisolvens—2_1_22, Parabacteroides distasonis—3_1_19, Bacteroides dorea—9_1_42FAA, Bacteroides ovatus—D2, Bacteroides stercoris—VPI B3-21, ATCC 43183, CIP 104203, JCM 9496, Bacteroides thetaiotaomicron—VPI 5482 [CIP 104206T, E50, NCTC 10582], Bacteroides uniformis—ATCC 8492, Bacteroides vulgatus—NCTC 11154, Bifidobacterium pseudocatenulatum—B1279, ATCC 27919, Bilophila wadsworthia—WAL 7959 [Lab 88-130H], Blautia hansenii—VPI C7-24, Blautia hydrogenotrophica—S5a33, Blautia obeum—ATCC 29174, KCTC 15206, VPI B3-21, Blautia sp.—KLE 1732, Blautia wexlerae—ATCC BAA-1564, JCM 17041, KCTC 5965, WAL 14507, Catenibacterium mitsuokai—RCA14-39, CIP 106738, JCM 10609, Clostridium asparagiforme—N6, CCUG 48471, Clostridium hylemonae—TN-271, JCM 10539, Clostridium leptum—VPI T7-24-1, ATCC 29065, Tyzzerella nexilis DSM 1787, Clostridium saccharolyticum—WM1, ATCC 35040, NRC 2533, Absiella dolichum DSM 3991, Collinsella aerofaciens—VPI 1003 [DSM 3979, JCM 10188], Collinsella stercoris—RCA 55-54, JCM 10641, Coprococcus comes—VPI CI-38, Dialister invisus—E7.25, CCUG 47026, Eubacterium rectale—VPI 0990 [CIP 105953], Eubacterium siraeum—VPI T9-50-2, ATCC 29066, DSM 3996, Eubacterium ventriosum—VPI 1013B, Coprococcus eutactus—VPI C33-22, Holdemanella biformis—VPI C17-5, ATCC 27806, KCTC 5969, Intestinibacter bartlettii—WAL 16138, ATCC BAA-827, CCUG 48940, Megasphaera sp.—Sanger 24, Sanger_24, Odoribacter splanchnicus—1651/6, ATCC 29572, CCUG 21054, CIP 104287, LMG 8202, NCTC 10825, Parabacteroides distasonis—NCTC 11152, Parabacteroides merdae—VPI T4-1, ATCC 43184, CCUG 38734, CIP 104202, JCM 9497, Parabacteroides sp.—D13, Granulicatella adiacens—GaD [CIP 103243, DSM 9848], Holdemania filiformis—VPI J1-31B-1, ATCC 51649, Hungatella hathewayi—1313, CCUG 43506, CIP 109440, MTCC 10951, Intestinimonas butyriciproducens—SRB-521-5-1, CCUG 63529, Solobacterium moorei—RCA59-74, CIP 106864, JCM 10645, Mitsuokella multacida—A 405-1, ATCC 27723, NCTC 10934, Olsenella uli—D76D-27C, ATCC 49627, CIP 109912, Parabacteroides johnsonii—M-165, CIP 109537, JCM 13406, Prevotella buccalis—HS4, ATCC 35310, NCDO 2354, Prevotella copri—CB7, JCM 13464, Roseburia inulinivorans—A2-194, CIP 109405, JCM 17584, NCIMB 14030, Clostridium sp.—VPI C48-50 (unassigned Clostridiales), Ruminococcus gauvreauii—CCRI-16110, CCUG 54292, JCM 14987, NML 060141, Ruminococcus lactaris—VPI X6-29, Ruminococcus torques—VPI B2-51, Alistipes putredinis—CCUG 45780, CIP 104286, ATCC 29800, Carlier 10203, VPI 3293, Alistipes senegalensis—CSUR P150, JCM 32779, JC50, Clostridium spiroforme—VPI C28-23-1A, ATCC 29900, NCTC 11211, Slackia exigua—S-7, ATCC 700122, JCM 11022, KCTC 5966, Bacteroides pectinophilus—N3, Butyrivibrio crossotus T9-40A, ATCC 29175, Subdoligranulum variabile—BI-114, CCUG 47106, Turicibacter sanguinis—MOL361, NCCB 100008, Bifidobacterium breve—S1, ATCC 15700, NCTC 11815, Bifidobacterium catenulatum—B669, ATCC 27539, CECT 7362, CIP 104175, DSM 20103, Butyricimonas virosa—MT12, CCUG 56611, JCM 15149, Streptococcus salivarius subsp. thermophilus—LMD-9, Dorea formicigenerans—VPI C8-13 [JCM 9500], Bacteroides plebeius—M12, Ruminococcus gnavus—VPI C7-9, Oscillibacter sp.—KLE 1728, Clostridium sp.—M62/1, Slackia heliotrinireducens—RHS 1, ATCC 29202, NCTC 11029, Desulfovibrio piger—VPI C3-23 [DSM 749], Clostridium methylpentosum—R2, ATCC 43829, Ethanoligenens harbinense—YUAN-3, CGMCC 1.5033, JCM 12961, Marvinbryantia formatexigens—I-52, CCUG 46960, Lactobacillus ruminis—E 194e, Clostridium bolteae—WAL 16351, [CCUG 46953], ATCC BAA-613, Song et al. 2003, Clostridium hiranonis—TO-931, JCM 10541, KCTC 15199, Clostridium scindens—VPI 13733, ATCC 35704, 19, Bacteroides xylanisolvens—XB1A, CCUG 53782, Clostridium sp.—L2-50, Clostridium orbiscindens—1_3_50AFAA, Alistipes shahii—WAL 8301, and Faecalibacterium prausnitzii—A2-165, JCM 31915.

19. The high complexity defined gut microbial community of any one of claims 1-18, wherein the defined gut microbial community comprises Acidaminococcus, Adlercreutzia, Akkermansia, Anaerostipes, Anaerotruncus, Bacteroides, Bifidobacterium, Bilophila, Blautia, Butyrivibrio, Clostridium, Collinsella, Coprococcus, Desulfovibrio, Eggerthella, Eubacterium, Faecalibacterium, Marvinbryantia, Mitsuokella, Odoribacter, Parabacteroides, Roseburia, Ruminococcus, Slackia, and Solobacterium.

20. The high complexity defined gut microbial community of any one of claims 1-19, wherein the defined gut microbial community comprises Acidaminococcus fermentans, Adlercreutzia equolifaciens, Akkermansia muciniphila, Anaerostipes caccae, Anaerotruncus colihominis, Bacteroides caccae, Bacteroides cellulosilyticus, Bacteroides dorei, Bacteroides eggerthii, Bacteroides fragilis, Bacteroides intestinalis, Bacteroides ovatus, Bacteroides pectinophilus, Bacteroides plebeius, Bacteroides stercoris, Bacteroides thetaiotaomicron, Bacteroides uniformis, Bacteroides vulgatus, Bifidobacterium breve, Bifidobacterium catenulatum, Bifidobacterium pseudocatenulatum, Bilophila wadsworthia, Blautia hansenii, Blautia hydrogenotrophica, Butyrivibrio crossotus, Clostridium asparagiforme, Clostridium hiranonis, Clostridium hylemonae, Clostridium leptum, Clostridium orbiscindens, Clostridium saccharolyticum, Clostridium scindens, Collinsella aerofaciens, Coprococcus comes, Desulfovibrio piger, Eggerthella lenta, Eubacterium rectale, Eubacterium siraeum, Eubacterium ventriosum, Faecalibacterium prausnitzii, Marvinbryantia formatexigens, Mitsuokella multacida, Odoribacter splanchnicus, Parabacteroides distasonis, Parabacteroides johnsonii, Parabacteroides merdae, Roseburia inulinivorans, Ruminococcus gnavus, Ruminococcus lactaris, Ruminococcus torques, Slackia exigua, and Solobacterium moorei.

21. The high complexity defined gut microbial community of any one of claims 1-20, wherein the defined gut microbial community comprises Acidaminococcus fermentans—VR4, Acidaminococcus sp.—D21, Adlercreutzia equolifaciens—FJC-B9, Akkermansia muciniphila—Muc [CIP 107961], Alistipes finegoldii—AHN 2437, Alistipes indistinctus—JCM 16068, YIT 12060, Alistipes onderdonkii—WAL 8169, Anaerobutyricum hallii—VPI B4-27, Anaerofustis stercorihominis—ATCC BAA-858, CCUG 47767, CIP 108481, WAL 14563, Anaerostipes caccae—L1-92, Anaerotruncus colihominis —277, Bacteroides caccae—VPI 3452A [CIP 104201T, JCM 9498], Bacteroides cellulosilyticus—CRE21, CCUG 44979, Bacteroides coprocola—M16, Bacteroides coprophilus—CB42, JCM 13818, Bacteroides dorei —175, Bacteroides dorei —5_1_36/D4, Bacteroides eggerthii—ATCC 27754, NCTC 11155, Bacteroides finegoldii —199, Bacteroides fragilis—3_1_12, Bacteroides intestinalis —341, Bacteroides ovatus—NCTC 11153, Bacteroides rodentium—ST28, CCUG 59334, JCM 16469, Bacteroides thetaiotaomicron—1_1_6, Bacteroides fragilis—2_1_16, Bacteroides xylanisolvens—2_1_22, Parabacteroides distasonis—3_1_19, Bacteroides dorea—9_1_42FAA, Bacteroides ovatus—D2, Bacteroides stercoris—VPI B3-21, ATCC 43183, CIP 104203, JCM 9496, Bacteroides thetaiotaomicron—VPI 5482 [CIP 104206T, E50, NCTC 10582], Bacteroides uniformis—ATCC 8492, Bacteroides vulgatus—NCTC 11154, Bifidobacterium pseudocatenulatum—B1279, ATCC 27919, Bilophila wadsworthia—WAL 7959 [Lab 88-130H], Blautia hansenii—VPI C7-24, Blautia hydrogenotrophica—S5a33, Blautia obeum—ATCC 29174, KCTC 15206, VPI B3-21, Blautia sp.—KLE 1732, Blautia wexlerae—ATCC BAA-1564, JCM 17041, KCTC 5965, WAL 14507, Catenibacterium mitsuokai—RCA14-39, CIP 106738, JCM 10609, Clostridium asparagiforme—N6, CCUG 48471, Clostridium hylemonae—TN-271, JCM 10539, Clostridium leptum—VPI T7-24-1, ATCC 29065, Tyzzerella nexilis DSM 1787, Clostridium saccharolyticum—WM1, ATCC 35040, NRC 2533, Absiella dolichum DSM 3991, Collinsella aerofaciens—VPI 1003 [DSM 3979, JCM 10188], Collinsella stercoris—RCA 55-54, JCM 10641, Coprococcus comes—VPI CI-38, Dialister invisus—E7.25, CCUG 47026, Eubacterium rectale—VPI 0990 [CIP 105953], Eubacterium siraeum—VPI T9-50-2, ATCC 29066, DSM 3996, Eubacterium ventriosum—VPI 1013B, Coprococcus eutactus—VPI C33-22, Holdemanella biformis—VPI C17-5, ATCC 27806, KCTC 5969, Intestinibacter bartlettii—WAL 16138, ATCC BAA-827, CCUG 48940, Megasphaera sp.—Sanger 24, Sanger 24, Odoribacter splanchnicus—1651/6, ATCC 29572, CCUG 21054, CIP 104287, LMG 8202, NCTC 10825, Parabacteroides distasonis—NCTC 11152, Parabacteroides merdae—VPI T4-1, ATCC 43184, CCUG 38734, CIP 104202, JCM 9497, Parabacteroides sp.—D13, Granulicatella adiacens—GaD [CIP 103243, DSM 9848], Holdemania filiformis—VPI J1-31B-1, ATCC 51649, Hungatella hathewayi—1313, CCUG 43506, CIP 109440, MTCC 10951, Intestinimonas butyriciproducens—SRB-521-5-1, CCUG 63529, Solobacterium moorei—RCA59-74, CIP 106864, JCM 10645, Mitsuokella multacida—A 405-1, ATCC 27723, NCTC 10934, Olsenella uli—D76D-27C, ATCC 49627, CIP 109912, Parabacteroides johnsonii—M-165, CIP 109537, JCM 13406, Prevotella buccalis—HS4, ATCC 35310, NCDO 2354, Prevotella copri—CB7, JCM 13464, Roseburia inulinivorans—A2-194, CIP 109405, JCM 17584, NCIMB 14030, Clostridium sp.—VPI C48-50 (unassigned Clostridiales), Ruminococcus gauvreauii—CCRI-16110, CCUG 54292, JCM 14987, NML 060141, Ruminococcus lactaris—VPI X6-29, Ruminococcus torques—VPI B2-51, Alistipes putredinis—CCUG 45780, CIP 104286, ATCC 29800, Carlier 10203, VPI 3293, Alistipes senegalensis—CSUR P150, JCM 32779, JC50, Clostridium spiroforme—VPI C28-23-1A, ATCC 29900, NCTC 11211, Slackia exigua—S-7, ATCC 700122, JCM 11022, KCTC 5966, Bacteroides pectinophilus—N3, Butyrivibrio crossotus—T9-40A, ATCC 29175, Subdoligranulum variabile—BI-114, CCUG 47106, Turicibacter sanguinis—MOL361, NCCB 100008, Bifidobacterium breve—S1, ATCC 15700, NCTC 11815, Bifidobacterium catenulatum—B669, ATCC 27539, CECT 7362, CIP 104175, DSM 20103, Butyricimonas virosa—MT12, CCUG 56611, JCM 15149, Streptococcus salivarius subsp. thermophilus—LMD-9, Dorea formicigenerans—VPI C8-13 [JCM 9500], Bacteroides plebeius—M12, Ruminococcus gnavus—VPI C7-9, Oscillibacter sp.—KLE 1728, Clostridium sp.—M62/1, Slackia heliotrinireducens—RHS 1, ATCC 29202, NCTC 11029, Desulfovibrio piger—VPI C3-23 [DSM 749], Clostridium methylpentosum—R2, ATCC 43829, Ethanoligenens harbinense—YUAN-3, CGMCC 1.5033, JCM 12961, Marvinbryantia formatexigens—I-52, CCUG 46960, Lactobacillus ruminis—E 194e, Clostridium bolteae—WAL 16351, [CCUG 46953], ATCC BAA-613, Song et al. 2003, Clostridium hiranonis—TO-931, JCM 10541, KCTC 15199, Clostridium scindens—VPI 13733, ATCC 35704, 19, Bacteroides xylanisolvens—XB1A, CCUG 53782, Clostridium sp.—L2-50, Clostridium orbiscindens—1_3_50AFAA, Alistipes shahii—WAL 8301, and Faecalibacterium prausnitzii—A2-165, JCM 31915.

22. The high-complexity defined gut microbial community according to any one of claims 1-21 wherein community stability is characterized by up to 10% of the defined microbial strains dropping out following the microbial challenge.

23. The high-complexity defined gut microbial community according to any one of claims 1-22, wherein community stability is characterized by the appearance of up to 10% of new strains contributed from the human fecal community appearing following the microbial challenge.

24. The high-complexity defined gut microbial community according to claim 1, wherein at least 50% of the defined microbial strains are detectable following the microbial challenge.

25. The high-complexity defined gut microbial community according to claim 24, wherein at least 60% of the defined microbial strains are detectable following the microbial challenge.

26. The high-complexity defined gut microbial community according to claim 25, wherein at least 70% of the defined microbial strains are detectable following the microbial challenge.

27. The high-complexity defined gut microbial community according to claim 26, wherein at least 80% of the defined microbial strains are detectable following the microbial challenge.

28. The high-complexity defined gut microbial community according to claim 27, wherein at least 90% of the defined microbial strains are detectable following the microbial challenge.

29. The high-complexity defined gut microbial community according to claim 28, wherein at least 95% of the defined microbial strains are detectable following the microbial challenge.

30. The high-complexity defined gut microbial community according to claim 29, wherein at least 99% of the defined microbial strains are detectable following the microbial challenge.

31. The high-complexity defined gut microbial community according to any one of claims 1-30, wherein community stability is characterized by metagenomic analysis of a fecal sample obtained from the mouse following the microbial challenge.

32. The high-complexity defined gut microbial community of claim 31, wherein metagenomic analysis is selected from whole genome sequencing, ribosomal gene sequencing, or ribosomal RNA sequencing.

33. The high-complexity defined gut microbial community of claim 32, wherein whole genome sequencing is whole genome shotgun sequencing.

34. The high-complexity defined gut microbial community according to any one of claims 1-33, wherein the defined gut microbial community comprises between 100 and 200 defined microbial strains.

35. The high-complexity defined gut microbial community according to claim 34, wherein the defined gut microbial community comprises between 100 and 150 defined microbial strains.

36. The high-complexity defined gut microbial community according to any one of claims 1-35, wherein each defined microbial strain is molecularly identified.

37. The high-complexity defined gut microbial community according to claim 36, wherein the molecular identification comprises identification of a nucleic acid sequence that uniquely identifies each of the defined microbial strains.

38. The high-complexity defined gut microbial community according to claim 37 wherein the nucleic acid sequence comprises a 16S rRNA sequence.

39. The high-complexity defined gut microbial community according to claim 37, wherein the nucleic acid sequence comprises a whole genomic sequence.

40. The high-complexity defined gut microbial community according to claim 36, wherein the molecular identification comprises Matrix-Assisted Laser Desorption/Ionization Time-Of-Flight Mass Spectrometry.

41. A method of treating an animal having a dysbiosis or pathological condition comprising administering a high-complexity defined gut microbial community according to any one of claims 1-40.

42. The method of claim 41, wherein the animal is a mammal.

43. The method of claim 42, wherein the animal is a human.

44. The method according to any one of claims 41-44, wherein the high-complexity defined gut microbial community is administered via a route selected from the group consisting of oral, rectal, fecal (by enema), and naso/oro-gastric gavage.

45. A method of making a high-complexity defined gut microbial community according to any one of claims 1-40, wherein each of the plurality of defined microbial strains is individually cultured then combined to form the defined gut microbial community.

46. A method of making a high-complexity defined gut microbial community according to any one of claims 1-40 wherein all of the plurality of defined microbial strains are cultured together to form the defined gut microbial community.

47. A method of making a high-complexity defined gut microbial community according to any one of claims 1-40, wherein one or more of the plurality of defined microbial strains is individually cultured and two or more of the defined microbial strains are cultured together, and wherein the individually cultured defined microbial strains and the co-cultured defined microbial strains are combined together to form the defined gut microbial community.

48. A formulation comprising the high-complexity defined gut microbial community according to any one of claims 1-40 and a pharmaceutically acceptable carrier or excipient.
